(19) United States
(12) Patent Application Publication
(10) Pub. No.: US 2024/0325409 A1
(43) Pub. Date: Oct. 3, 2024

(54) Fused Heterotetracyclic Antiviral Agents

(71) Applicant: Enanta Pharmaceuticals, Inc., Watertown, MA (US)

(72) Inventors: Sean RAFFERTY, Watertown, MA (US); Sourav GHORAI, Waltham, MA (US); Nathaniel Thomas KENTON, Watertown, MA (US); Peilin XU, Watertown, MA (US); Samuel BARTLETT, Brighton, MA (US); Scott MITCHELL, Woburn, MA (US); Joseph D. PANARESE, Newton, MA (US); Yat Sun OR, Waltham, MA (US)

(21) Appl. No.: 18/587,048

(22) Filed: Feb. 26, 2024

Related U.S. Application Data
(60) Provisional application No. 63/448,398, filed on Feb. 27, 2023.

(51) Int. Cl. A61K 31/554; A61K 45/06; A61P 31/20; C07D 513/04

(52) U.S. Cl. CPC A61K 31/554; A61K 45/06; A61P 31/20; C07D 513/04

(57) ABSTRACT

The present invention discloses compounds of Formula (I), or pharmaceutically acceptable salts, thereof:

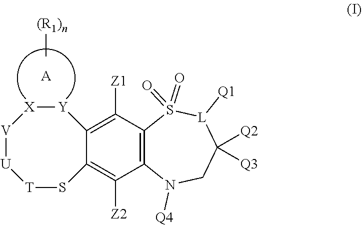

which inhibit the cellular entry of hepatitis B virus (HBV) and/or hepatitis D virus (HDV) or interfere with the function of the life cycle of HBV and/or HDV and are also useful as antiviral agents. The present invention further relates to pharmaceutical compositions comprising the aforementioned compounds for administration to a subject suffering from HBV and/or HDV infection. The invention also relates to methods of treating an HBV and/or HDV infection in a subject by administering a pharmaceutical composition comprising the compounds of the present invention.

### RELATED APPLICATION

This application claims the benefit of U.S. Provisional Application No. 63/448,398, filed on Feb. 27, 2023. The entire teachings of the above application are incorporated herein by reference.

### TECHNICAL FIELD

The present invention relates generally to compounds and pharmaceutical compositions useful as hepatitis virus inhibitors. Specifically, the present invention relates to benzothiadiazepine compounds that are useful in treating viral infections such as hepatitis B virus (HBV) and/or hepatitis D virus (HDV). These compounds can function through inhibition of the Na⁺-taurocholate cotransporting polypeptide (NTCP) receptor. The invention provides novel benzothiadiazepine compounds as disclosed herein, pharmaceutical compositions containing such compounds, and methods of using these compounds and compositions in the treatment and prevention of HBV and/or HDV infections.

### BACKGROUND OF THE INVENTION

The hepatitis delta viruses, or HDV, are eight species of negative-sense single-stranded RNA viruses (or virus-like particles) classified together as the genus Deltavirus, within the realm Ribozyviria. The HDV virion is a small, spherical, enveloped particle with a 36 nm diameter; its viral envelope contains host phospholipids, as well as three proteins taken from the hepatitis B virus—the large, medium, and small hepatitis B surface antigens. This assembly surrounds an inner ribonucleoprotein (RNP) particle, which contains the genome surrounded by hepatitis D antigen (HDAg).

The HDV genome is negative sense, single-stranded, closed circular RNA; with a genome of approximately 1700 nucleotides, HDV is the smallest virus known to infect animals. Its genome is unique among animal viruses because of its high GC nucleotide content. Its nucleotide sequence is about 70% self-complementary, allowing the genome to form a partially double-stranded, rod-like RNA structure. Millions of people throughout the world are chronically infected with hepatitis D virus (HDV). For those that are chronically infected, many will develop complications of liver disease from cirrhosis or hepatocellular carcinoma (HCC).

HBV is a member of the Hepadnavirus family, and it is able to replicate through the reverse transcription of an RNA intermediate. The 3.2-kb HBV genome exists in a circular, partially doublestranded DNA conformation (rcDNA) that has four overlapping open reading frames (ORF). These encode for the core, polymerase, envelope, and X proteins of the virus. rcDNA must be converted into covalently closed circular DNA (cccDNA) in cells prior to the transcription of viral RNAs. As rcDNA is transcriptionally inert, cccDNA is the only template for HBV transcription, and its existence is required for infection.

The HBV viral envelope contains a mixture of surface antigen proteins (HBsAg). The HBsAg coat contains three proteins that share a common region that includes the smallest of the three proteins (SHBsAg). The other two proteins, Medium HBsAg (MHBsAg) and Large HBsAg (LHBsAg), both contain a segment of SHBsAg with additional polypeptide segments. SHBsAg, MHBsAg, and LHBsAg can also assemble into a non-infectious subviral particle known as the 22-nm particle that contains the same proteins found around infectious viral particles. As the 22-nm particles contain the same antigenic surface proteins that exist around the infectious HBV virion, they can be used as a vaccine to produce neutralizing antibodies.

HBV and HDV both gain entry into liver cells via the human NTCP bile acid transporter. Viral particles recognize their receptor via the N-terminal domain of the large hepatitis B surface antigen, HBsAg. After entering the hepatocyte, the virus is uncoated and the nucleocapsid translocated to the nucleus thereby infecting the cell.

There is a need in the art for novel therapeutic agents that treat, ameliorate or prevent HBV and/or HDV infection. Administration of these therapeutic agents to an HBV and/or HDV infected patient, either as monotherapy or in combination with other HBV and/or HDV treatments or ancillary treatments, will lead to significantly improved prognosis, diminished progression of the disease, and enhanced seroconversion rates.

### SUMMARY OF THE INVENTION

The present invention relates to novel antiviral compounds, pharmaceutical compositions comprising such compounds, as well as methods to treat or prevent viral (particularly HBV and/or HDV) infection in a subject in need of such therapy with said compounds. Compounds of the present invention inhibit the entry of HBV and/or HDV or interfere with the life cycle of HBV and/or HDV and are also useful as antiviral agents. In addition, the present invention provides processes for the preparation of said compounds.

The present invention provides compounds represented by Formula (I),

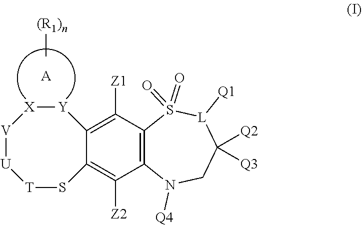

and pharmaceutically acceptable salts, N-oxides, esters and prodrugs thereof, wherein:
L is CR₁₄ or N;
R₁₄ is hydrogen, optionally substituted —C₁-C₆ alkyl, optionally substituted —C₂-C₆ alkenyl, optionally substituted —C₂-C₆ alkynyl, or optionally substituted —C₁-C₆ alkoxy;
when L is N, Q1 is selected from the group consisting of hydrogen, optionally substituted —C₁-C₆ alkyl, optionally substituted —C₂-C₆ alkenyl, optionally substituted —C₃-C₈ cycloalkyl, optionally substituted —C₃-C₈ cycloalkenyl, optionally substituted 3- to 8-membered heterocycloalkyl, optionally substituted aryl, optionally substituted heteroaryl, optionally substituted arylalkyl, and optionally substituted heteroarylalkyl;
when L is CR₁₄, Q1 is selected from the group consisting of hydrogen, optionally substituted —C₁-C₆ alkyl, optionally substituted —C₂-C₆ alkenyl, optionally substituted —C₁-C₆ alkoxy, optionally substituted —C₃-C₈ cycloalkyl, optionally substituted —C₃-C₈ cycloalkenyl, optionally substituted 3- to 8-membered heterocycloalkyl, optionally substituted aryl, optionally substituted heteroaryl; optionally substituted arylalkyl, and optionally substituted heteroarylalkyl;
Q2 and Q3 are each independently selected from the group consisting of hydrogen, optionally substituted —C₁-C₆ alkyl, optionally substituted —C₂-C₆ alkenyl, optionally substituted —C₁-C₆ alkoxy, optionally substituted —C₃-C₈ cycloalkyl, optionally substituted —C₃-C₈ cycloalkenyl, optionally substituted 3- to 8-membered heterocycloalkyl, optionally substituted aryl, optionally substituted heteroaryl; optionally substituted arylalkyl, and optionally substituted heteroarylalkyl;
Q4 is selected from the group consisting of hydrogen, optionally substituted —C₁-C₆ alkyl, optionally substituted —C₂-C₆ alkenyl, optionally substituted —C₃-C₈ cycloalkyl, optionally substituted —C₃-C₈ cycloalkenyl, optionally substituted 3- to 8-membered heterocycloalkyl, optionally substituted aryl, optionally substituted heteroaryl; optionally substituted arylalkyl, and optionally substituted heteroarylalkyl;
Z1 and Z2 are each independently selected from the group consisting of:
1) hydrogen;
2) halogen;
3) —NO₂;
4) Cyano;
5) Optionally substituted —C₁-C₈ alkyl;
6) Optionally substituted —C₂-C₈ alkenyl;
7) Optionally substituted —C₂-C₈ alkynyl;
8) Optionally substituted —C₃-C₈ cycloalkyl;
9) Optionally substituted 3- to 12-membered heterocycloalkyl;
10) Optionally substituted aryl;
11) Optionally substituted arylalkyl;
12) Optionally substituted heteroaryl;
13) Optionally substituted heteroarylalkyl;
14) —SR₁₁;
15) —S(O)₂R₁₁;
16) —S(O)₂N(R₁₁)(R₁₂);
17) —C(O)R₁₁;
18) —C(O)OR₁₁;
19) —C(O)N(R₁₁)(R₁₂);
20) —C(O)N(R₁₁)S(O)₂(R₁₂);
21) —N(R₁₁)(R₁₂);
22) —N(R₁₃)C(O)N(R₁₁)(R₁₂);
23) —N(R₁₁)C(O)(R₁₂);
24) —N(R₁₁)C(O)₂(R₁₂);
25) —N(R₁₃)S(O)₂N(R₁₁)(R₁₂);
26) —N(R₁₁)S(O)₂(R₁₂);
27) —OR₁₁;
28) —OC(O)R₁₁;
29) —OC(O)OR₁₁; and
30) —OC(O)N(R₁₁)(R₁₂);
   wherein Ru, R₁₂, and R₁₃ are each independently selected from the group consisting of hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, and optionally substituted heteroaryl;
   alternatively, Ru and R₁₂ are taken together with the nitrogen atom to which they attached to form an optionally substituted 3-8 membered heterocyclic containing 0, 1, 2, or 3 double bonds;
ring A is a 6- to 12-membered aromatic ring or a 5- to 12-membered heteroaromatic ring containing 1-3 atoms independently selected from N, O, and S wherein X and Y are the atoms at the site of ring fusion;
X is C or N;
Y is C or N;
two of S, T, U and V are selected from the group consisting of O, C(R₁₅)(R₁₆), N(R₁₇), S, S(O), S(O)₂, C(O); and the remainder of S, T, U and V are independently C(R₁₅)(R₁₆); one, two, or all of U, V, and T can be absent;
   wherein
R₁₅ and R₁₆, are each independently selected from the group consisting of hydrogen, optionally substituted —C₁-C₈ alkyl, halogen, hydroxy, optionally substituted —C₁-C₆ alkoxy, optionally substituted —C₂-C₈ alkenyl, optionally substituted —C₃-C₈ cycloalkyl, optionally substituted 3- to 8-membered heterocycloalkyl, optionally substituted aryl, and optionally substituted heteroaryl;
R₁₇ is selected from hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, and optionally substituted heteroaryl; alternatively, R₁₅ and R₁₆, are taken together with the carbon atom to which they are attached to form an optionally substituted 3-8 membered heterocyclic or carbocyclic ring containing 0, 1, 2, or 3 double bonds;
n is 0, 1, 2, 3, or 4;
each R₁ is independently selected from the group consisting of:
1) halogen;
2) —NO₂;
3) Cyano;
4) Optionally substituted —C₁-C₈ alkyl;
5) Optionally substituted —C₂-C₈ alkenyl;
6) Optionally substituted —C₂-C₈ alkynyl;
7) Optionally substituted —C₃-C₈ cycloalkyl;
8) Optionally substituted 3- to 12-membered heterocycloalkyl;
9) Optionally substituted aryl;
10) Optionally substituted arylalkyl;
11) Optionally substituted heteroaryl;
12) Optionally substituted heteroarylalkyl;
13) —SR₂₇;
14) —SO₃H;
15) —S(O)₂R₂₇;
16) —S(O)₂N(R₂₇)(R₂₈);
17) —PO₃H₂;
18) —C(O)R₂₇;
19) —C(O)OR₂₇;
20) —C(O)N(R₂₇)(R₂₈);
21) —C(O)N(R₂₇)S(O)₂(R₂₈);
22) —N(R₂₇)(R₂₈);
23) —N(R₂₉)C(O)N(R₂₇)(R₂₈);
24) —N(R₂₇)C(O)(R₂₈);
25) —N(R₂₇)C(O)₂(R₂₈);
26) —N(R₂₉)S(O)₂N(R₂₇)(R₂₈);
27) —N(R₂₇)S(O)₂(R₂₈);
28) —OR₂₇;
29) —OC(O)R₂₇;
30) —OC(O)OR₂₇; and
31) —OC(O)N(R₂₇)(R₂₈);
   wherein R₂₇, R₂₈, R₂₉, are each independently selected from the group consisting of hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, and optionally substituted heteroaryl;
alternatively, R₂₇ and R₂₈ are taken together with the nitrogen atom to which they are attached to form an optionally substituted 3-8 membered heterocyclic containing 0, 1, 2, or 3 double bonds; and
each preferred group stated above can be taken in combination with one, any or all other preferred groups.

### DETAILED DESCRIPTION OF THE INVENTION

In one embodiment, the present invention provides a compound of Formula (I) as described above, or a pharmaceutically acceptable salt thereof.

In certain embodiments of the compounds of Formula (I), Z1 is hydrogen, halogen, -Me, or —OMe.

In certain embodiments of the compounds of Formula (I), Z1 is hydrogen.

In certain embodiments of the compounds of Formula (I), Z2 is hydrogen, halogen, -Me, or —OMe.

In certain embodiments of the compounds of Formula (I), Z2 is hydrogen.

In certain embodiments of the compounds of Formula (I), Z1 is hydrogen, and Z2 is hydrogen.

In certain embodiments of the compounds of Formula (I), L is nitrogen.

In certain embodiments of the compounds of Formula (I), L is nitrogen and Q1 is hydrogen or optionally substituted methyl.

In certain embodiments of the compounds of Formula (I), L is CR₁₄, where R₁₄ is hydrogen, methyl, ethyl, isopropyl or cyclopropyl.

In certain embodiments of the compounds of Formula (I), L is CR₁₄, where R₁₄ is hydrogen, methyl, ethyl, isopropyl or cyclopropyl, and Q1 is hydrogen, or optionally substituted methyl.

In certain embodiments of the compounds of Formula (I), Q2 is hydrogen, or optionally substituted methyl.

In certain embodiments of the compounds of Formula (I), Q3 is optionally substituted —C₁-C₆ alkyl, optionally substituted —C₃-C₈ cycloalkyl, optionally substituted 3- to 8-membered heterocycloalkyl, optionally substituted aryl, or optionally substituted heteroaryl.

In certain embodiments of the compounds of Formula (I), Q3 is —CH₂R₃₀, wherein R₃₀ is optionally substituted —C₁-C₈ alkyl, optionally substituted —C₂-C₅ alkenyl, optionally substituted —C₁-C₈ alkoxy, optionally substituted —C₃-C₈ cycloalkyl, optionally substituted —C₃-C₈ cycloalkenyl, optionally substituted 3- to 8-membered heterocycloalkyl, optionally substituted aryl, or optionally substituted heteroaryl.

In certain embodiments of the compounds of Formula (I), Q3 is optionally substituted phenyl, optionally substituted benzyl, optionally substituted methyl, optionally substituted ethyl, optionally substituted n-butyl, optionally substituted t-butyl, optionally substituted isopropyl, optionally substituted isobutyl, optionally substituted neopentyl,

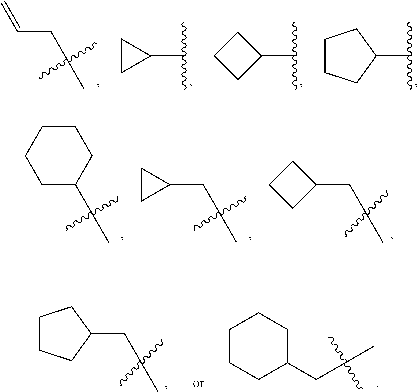

In certain embodiments of the compounds of Formula (I), Q4 is hydrogen or optionally substituted —C₁-C₆ alkyl.

In certain embodiments of the compounds of Formula (I), Q4 is optionally substituted —C₃-C₈ cycloalkyl or optionally substituted 3- to 8-membered heterocycloalkyl.

In certain embodiments of the compounds of Formula (I), Q4 is optionally substituted aryl or optionally substituted heteroaryl.

In certain embodiments of the compounds of Formula (I), Q4 is optionally substituted phenyl.

In certain embodiments of the compounds of Formula (I), Q4 is derived from one of the following by removal of a hydrogen atom and is optionally substituted:

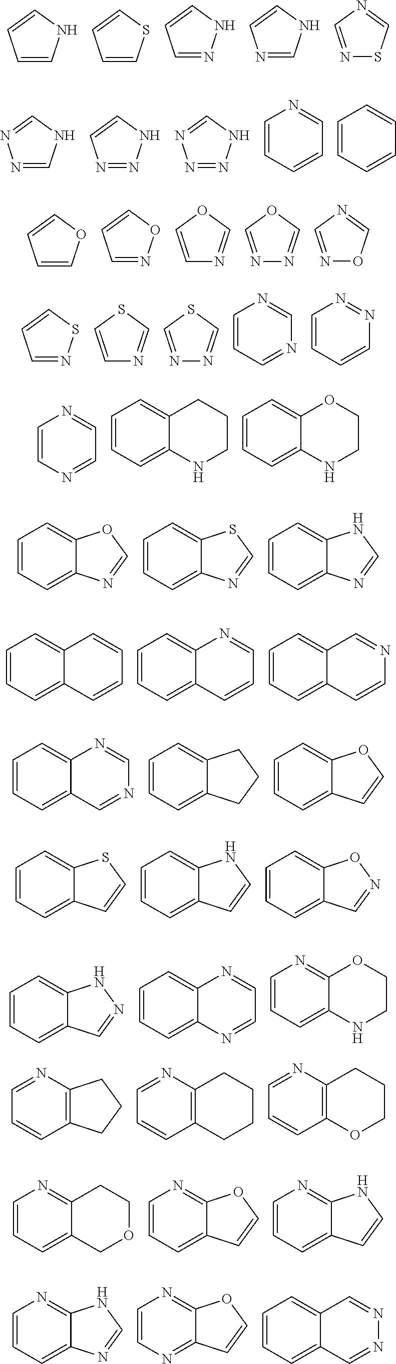

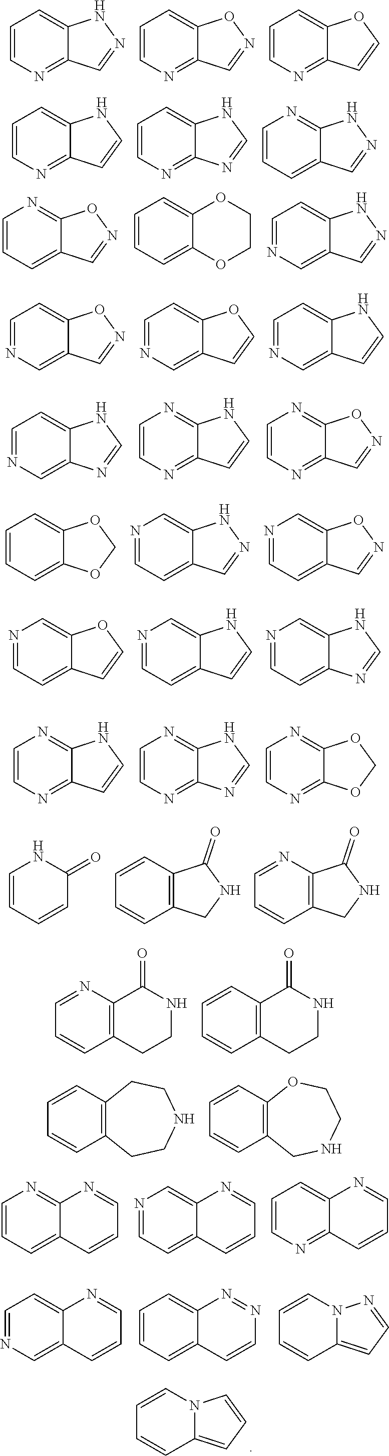

In certain embodiments of the compounds of Formula (I), Q4 is derived from one of the following by removal of a hydrogen atom and is optionally substituted:

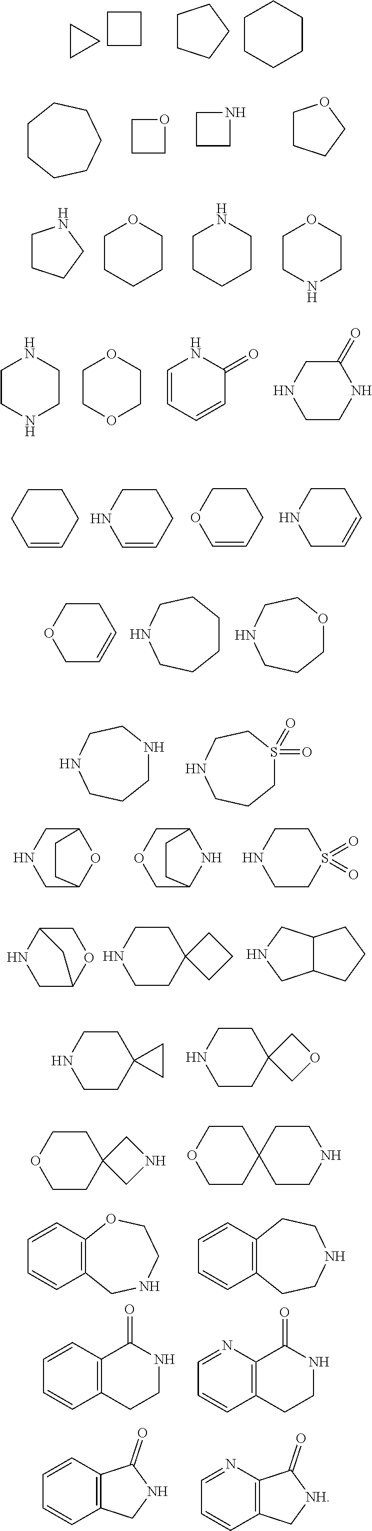

In certain embodiments of the compounds of Formula (I), X is carbon.

In certain embodiments of the compounds of Formula (I), Y is carbon.

In certain embodiments of the compounds of Formula (I), X and Y are both carbon.

In certain embodiments of the compounds of Formula (I), A is a six-membered heteroaromatic group and X and Y are both carbon.

In certain embodiments of the compounds of Formula (I), A is a five-membered heteroaromatic group and no more than one of X and Y is nitrogen.

In certain embodiments of the compounds of Formula (I), A is phenyl or 5- or 6-membered heteroaryl.

In certain embodiments of the compounds of Formula (I), n is 1, 2, or 3, and each R₁ is independently selected from —COOH, halogen, and optionally substituted methyl.

In certain embodiments of the compounds of Formula (I), n is 1 and R₁ is —COOH.

In certain embodiments of the compounds of Formula (I), n is 2, one R₁ is halogen, preferably fluorine and the other R₁ is —COOH. In certain embodiments of the compounds of Formula (I), n is 3, two R₁ are independently halogen, preferably fluorine, and the other R₁ is —COOH.

In certain embodiments of the compounds of Formula (I), S, T, U, and V have the definitions set forth in each entry in the table below:

| Entry | S | T | U | V |
|---|---|---|---|---|
| 1 | C(R₁₅)(R₁₆) | O | absent | absent |
| 2 | O | C(R₁₅)(R₁₆) | absent | absent |
| 3 | C(R₁₅)(R₁₆) | N(R₁₇) | absent | absent |
| 4 | N(R₁₇) | C(O) | absent | absent |
| 5 | C(O) | N(R₁₇) | absent | absent |
| 6 | C(R₁₅)(R₁₆) | S | absent | absent |
| 7 | S | C(R₁₅)(R₁₆) | absent | absent |
| 8 | C(R₁₅)(R₁₆) | S(O) | absent | absent |
| 9 | S(O) | C(R₁₅)(R₁₆) | absent | absent |
| 10 | C(R₁₅)(R₁₆) | S(O)₂ | absent | absent |
| 11 | S(O)₂ | C(R₁₅)(R₁₆) | absent | absent |
| 12 | S(O)₂ | N(R₁₇) | absent | absent |
| 13 | N(R₁₇) | S(O)₂ | absent | absent |
| 14 | C(R₁₅)(R₁₆) | C(R₁₅)(R₁₆) | O | absent |
| 15 | O | C(R₁₅)(R₁₆) | C(R₁₅)(R₁₆) | absent |
| 16 | C(R₁₅)(R₁₆) | O | C(R₁₅)(R₁₆) | absent |
| 17 | C(R₁₅)(R₁₆) | C(R₁₅)(R₁₆) | N(R₁₇) | absent |
| 18 | N(R₁₇) | C(R₁₅)(R₁₆) | C(R₁₅)(R₁₆) | absent |
| 19 | C(R₁₅)(R₁₆) | N(R₁₇) | C(R₁₅)(R₁₆) | absent |
| 20 | C(R₁₅)(R₁₆) | C(O) | N(R₁₇) | absent |
| 21 | N(R₁₇) | C(O) | C(R₁₅)(R₁₆) | absent |
| 22 | C(O) | N(R₁₇) | C(R₁₅)(R₁₆) | absent |
| 23 | C(R₁₅)(R₁₆) | N(R₁₇) | C(O) | absent |
| 24 | C(R₁₅)(R₁₆) | C(R₁₅)(R₁₆) | S | absent |
| 25 | S | C(R₁₅)(R₁₆) | C(R₁₅)(R₁₆) | absent |
| 26 | C(R₁₅)(R₁₆) | S | C(R₁₅)(R₁₆) | absent |
| 27 | C(R₁₅)(R₁₆) | C(R₁₅)(R₁₆) | S(O)₂ | absent |
| 28 | S(O)₂ | C(R₁₅)(R₁₆) | C(R₁₅)(R₁₆) | absent |
| 29 | C(R₁₅)(R₁₆) | S(O)₂ | C(R₁₅)(R₁₆) | absent |
| 30 | O | C(R₁₅)(R₁₆) | C(R₁₅)(R₁₆) | O |
| 31 | N(R₁₇) | C(R₁₅)(R₁₆) | C(R₁₅)(R₁₆) | O |
| 32 | O | C(R₁₅)(R₁₆) | C(R₁₅)(R₁₆) | N(R₁₇) |
| 33 | O | absent | absent | absent |
| 34 | N(R₁₇) | absent | absent | absent |
| 35 | S | absent | absent | absent |
| 36 | S(O)₂ | absent | absent | absent |

wherein R₁₅, R₁₆, and R₁₇ are as previously defined; each R₁₅, R₁₆, and R₁₇ can be the same or different; alternatively, R₁₅ and R₁₆ are taken together with the carbon atom to which they are attached to form an optionally substituted 3-8 membered heterocyclic or carbocyclic ring containing 0, 1, 2, or 3 double bonds. In certain embodiments, each R₁₅ and R₁₆ is hydrogen and R₁₇ is hydrogen or methyl.

In certain embodiments, the compound of Formula (I) is represented by Formula (I-a),

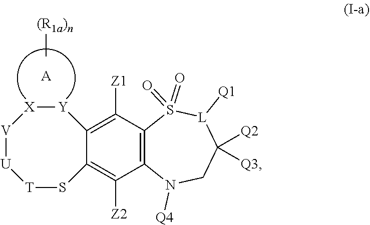

wherein each R₁ₐ is independently selected from the group consisting of:
1) halogen;
2) Cyano;
3) Optionally substituted —C₁-C₈ alkyl;
4) Optionally substituted —C₃-C₈ cycloalkyl;
5) —SR₂₇;
6) —SO₃H;
7) —S(O)₂R₂₇;
8) —S(O)₂N(R₂₇)(R₂₈);
9) —PO₃H₂;
10) —C(O)OR₂₇;
11) —C(O)N(R₂₇)(R₂₈);
12) —C(O)N(R₂₇)S(O)₂(R₂₈);
13) —N(R₂₇)(R₂₈);
14) —N(R₂₉)C(O)N(R₂₇)(R₂₈);
15) —N(R₂₇)C(O)(R₂₈);
16) —N(R₂₇)C(O)₂(R₂₈);
17) —N(R₂₉)S(O)₂N(R₂₇)(R₂₈);
18) —N(R₂₇)S(O)₂(R₂₈);
19) —OR₂₇; and
20) —OC(O)R₂₇;
R₂₇, R₂₈, Q1, Q2, Q3, Q4, Z1, Z2, S, T, U, V, X, Y, A, n and L are as previously defined.

In certain embodiments, the compound of Formula (I) is represented by Formula (II),

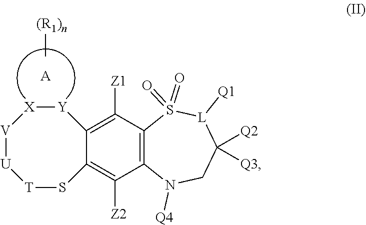

wherein Q1, Q2, Q3, Q4, Z1, Z2, S, T, U, V, X, Y, A, n and R₁ are as previously defined.

In certain embodiments, the compound of Formula (I) is represented by Formula (III),

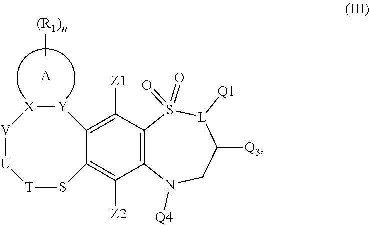

wherein L, Q1, Q3, Q4, Z1, Z2, S, T, U, V, X, Y, A, n and R₁ are as previously defined.

In certain embodiments, the compound of Formula (I) is represented by Formula (IV),

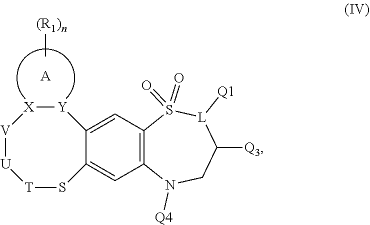

wherein L, Q1, Q3, Q4, S, T, U, V, X, Y, A, n and R₁ are as previously defined.

In certain embodiments, the compound of Formula (I) is represented by Formula (V-1) or Formula (V-2),

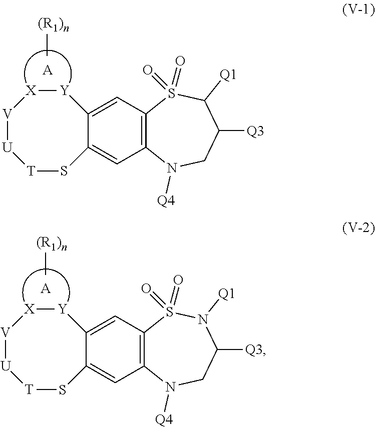

wherein Q1, Q3, Q4, S, T, U, V, X, Y, A, n and R₁ are as previously defined.

In certain embodiments, the compound of Formula (I) is represented by one of Formulae (VI-1)˜ (VI-23),

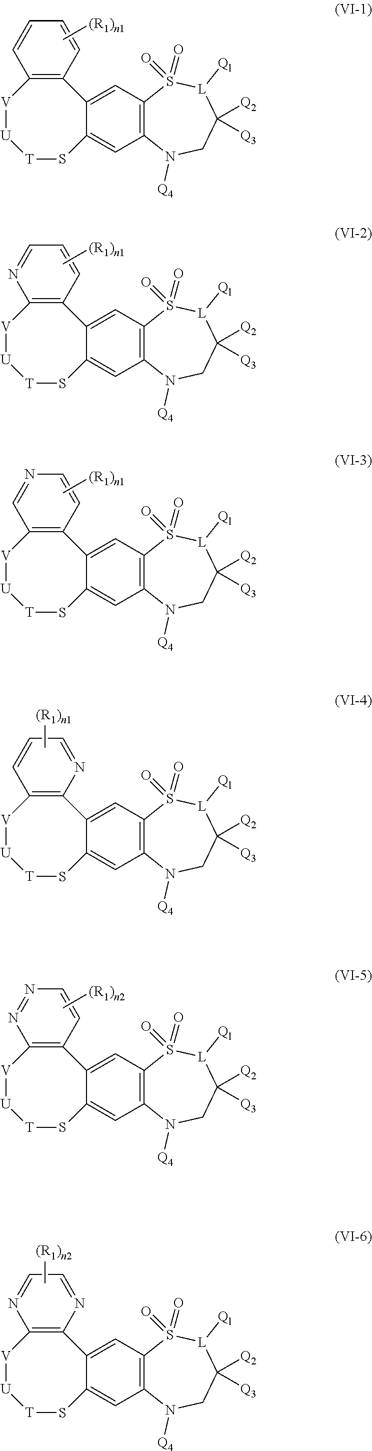

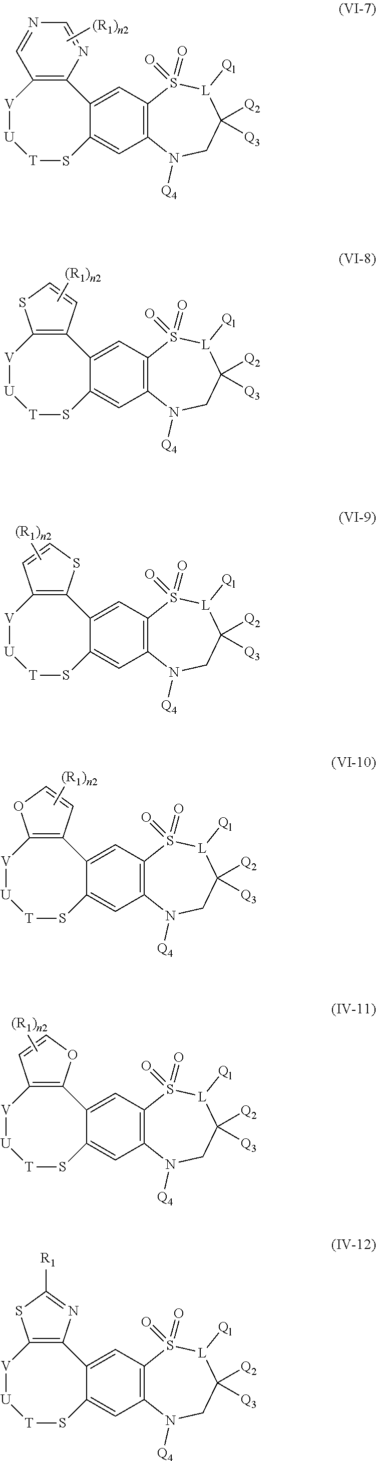

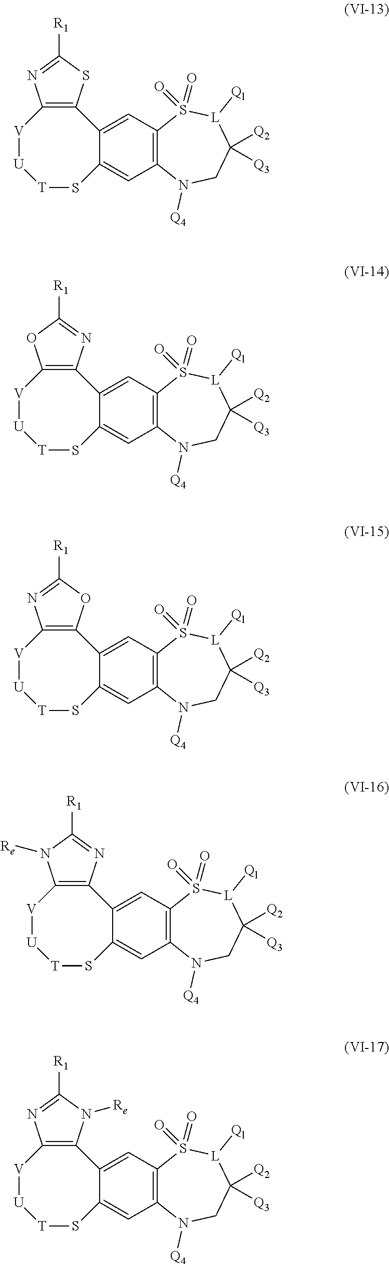

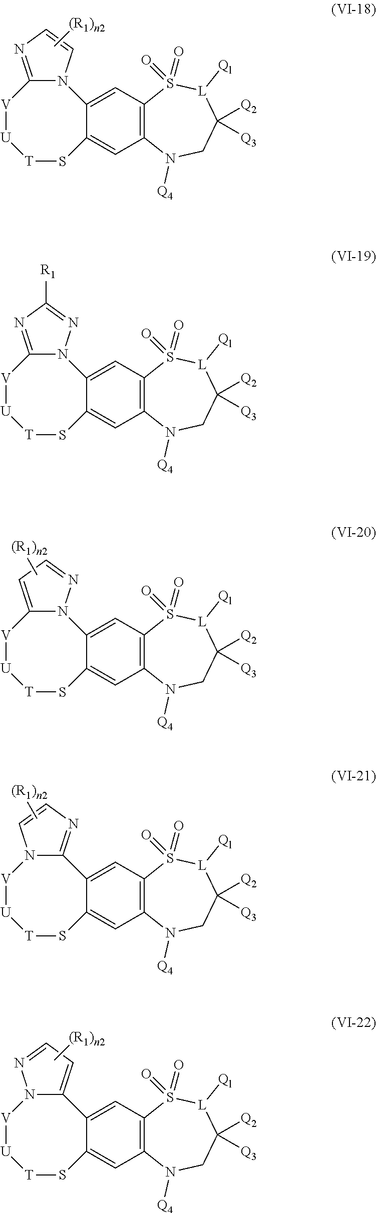

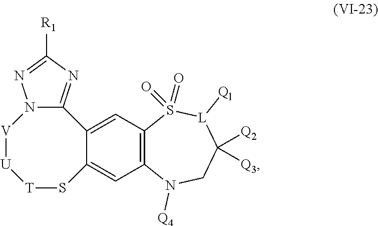

wherein n1 is 0, 1, 2 or 3; n2 is 0, 1 or 2; Rₑ is selected from the group consisting of:
1) hydrogen;
2) optionally substituted —C₁-C₆ alkyl;
3) optionally substituted —C₃-C₈ cycloalkyl;
4) optionally substituted 3- to 8-membered heterocycloalkyl;
5) optionally substituted aryl; and
6) optionally substituted heteroaryl;
   and R₁, L, Q1, Q2, Q3, Q4, S, T, U, V are as previously defined.

In certain embodiments, the compound of Formula (I) is represented by one of Formulae (VI-1a)˜(VI-23a),

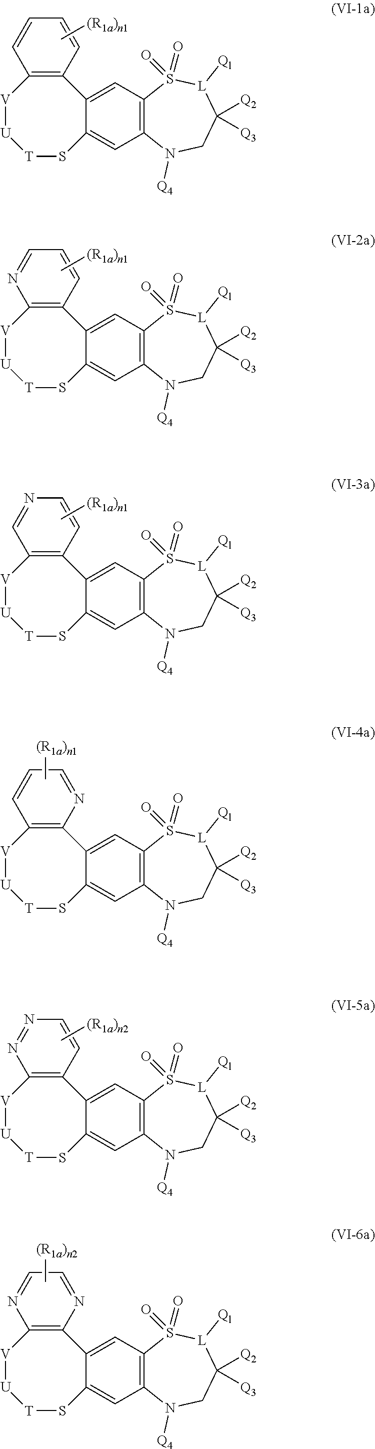

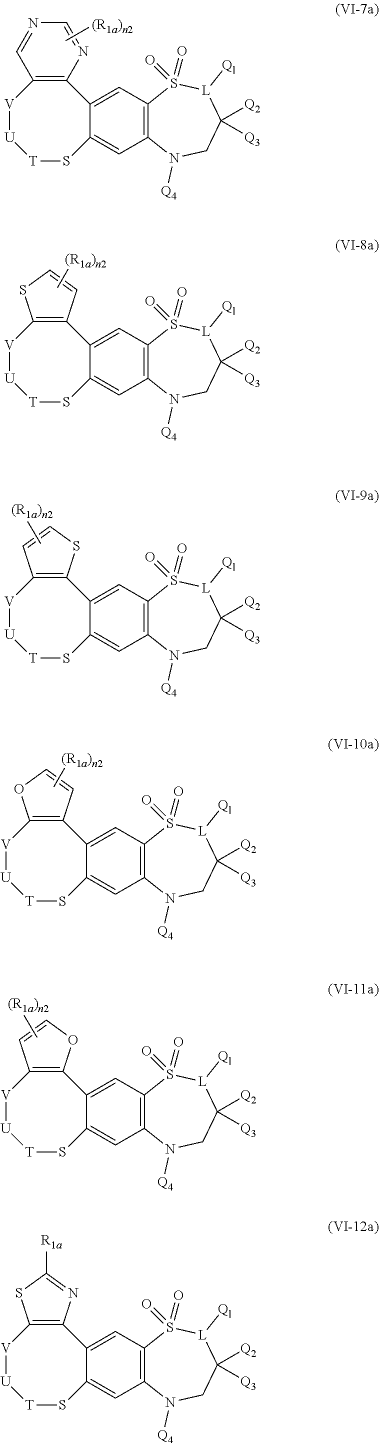

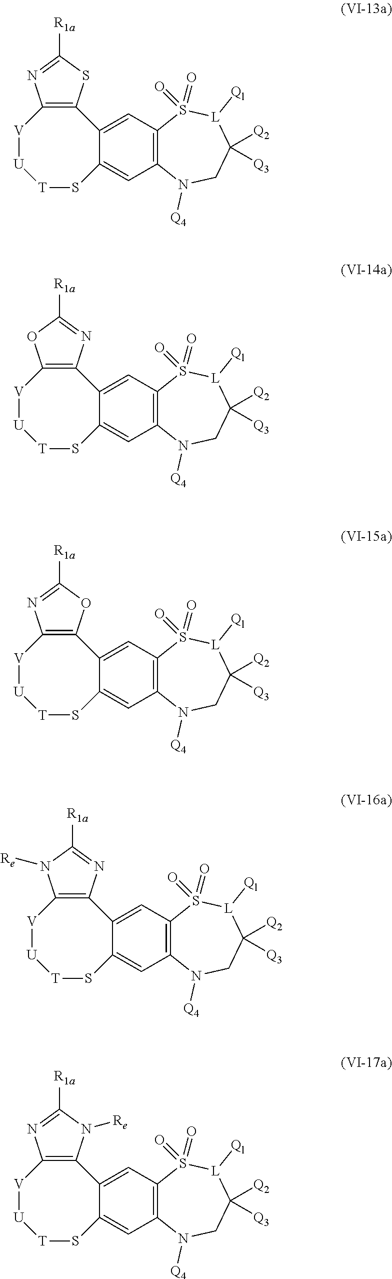

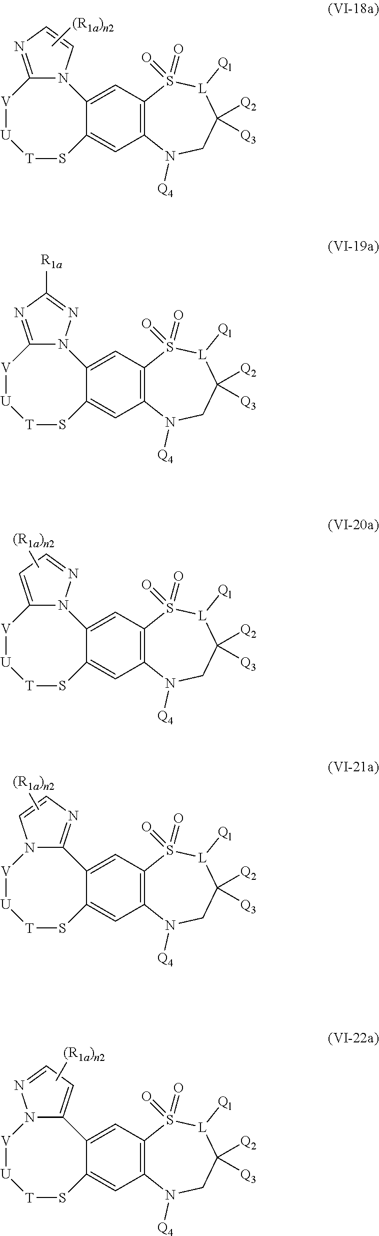

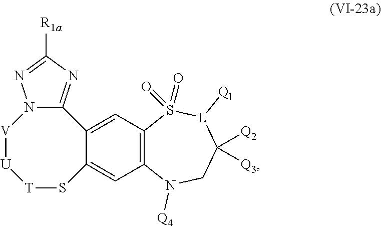

wherein n1, n2, Rₑ, R₁ₐ, L, Q1, Q2, Q3, Q4, S, T, U, and V are as previously defined.

In certain embodiments, the compound of Formula (I) is represented by one of Formulae (VI-1b)˜(VI-23b),

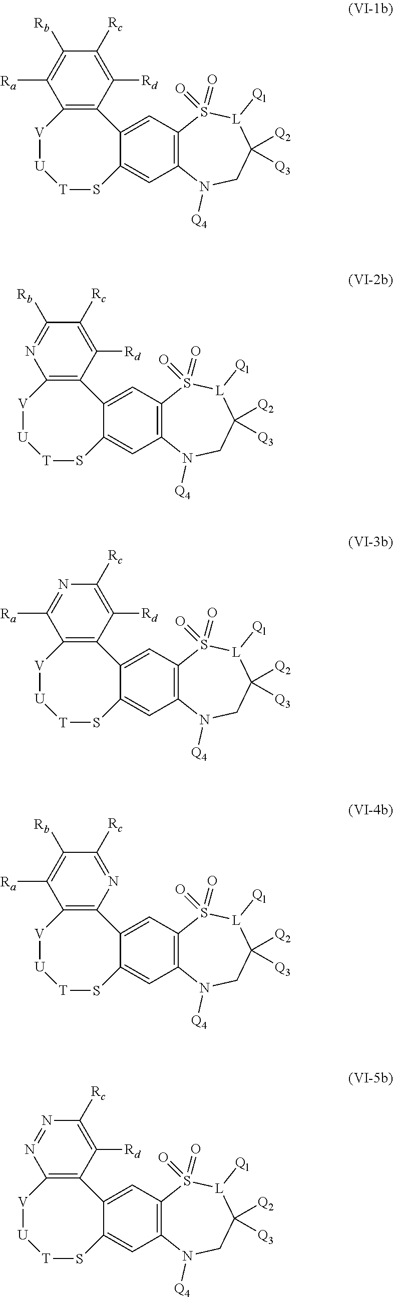

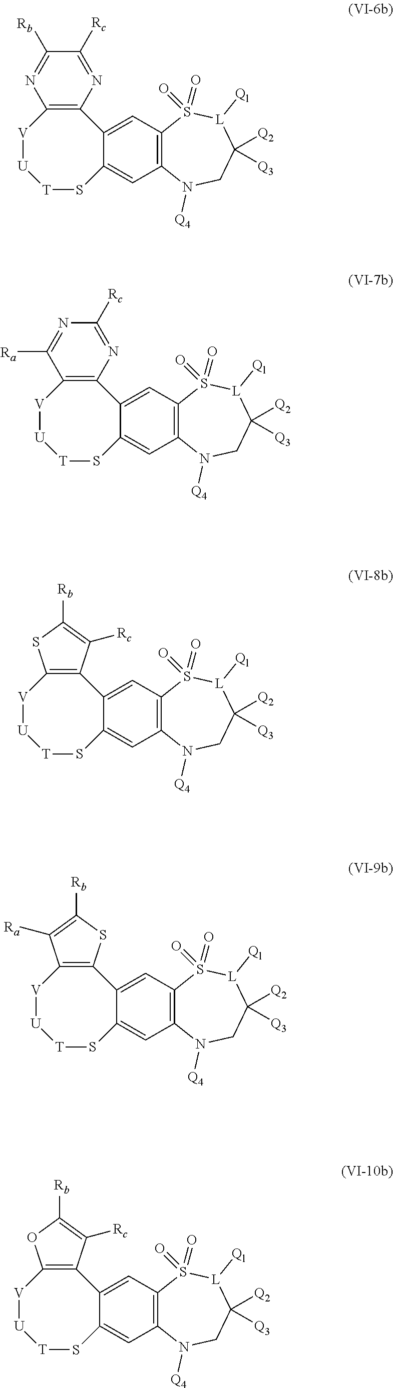

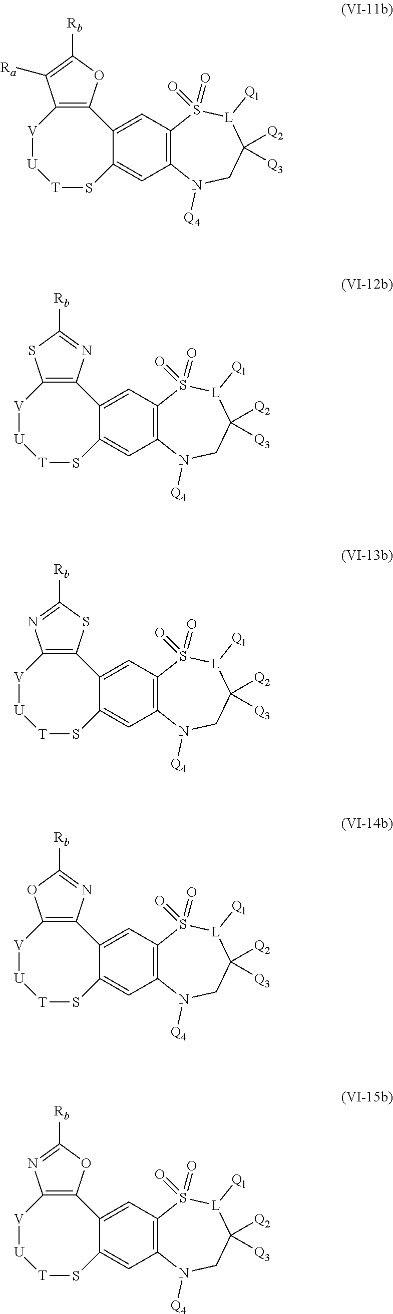

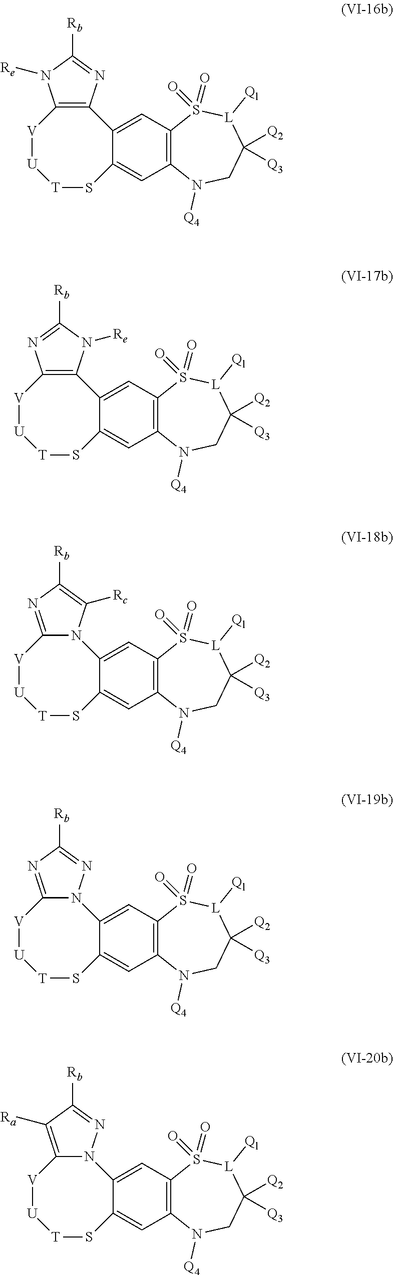

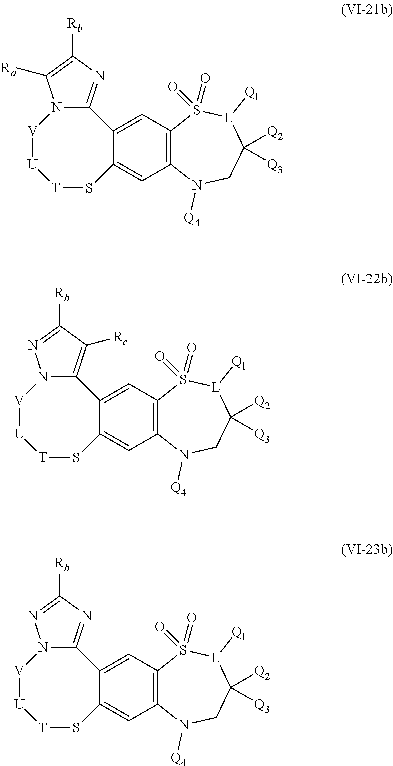

wherein Rₐ, R_{b}, R_{c}, and R_{d} are each independently selected from the group consisting of:
1) hydrogen;
2) halogen;
3) Cyano;
4) Optionally substituted —C₁-C₈ alkyl;
5) Optionally substituted —C₂-C₈ alkenyl;
6) —SR₂₇;
7) —SO₃H;
8) —S(O)₂R₂₇;
9) —S(O)₂N(R₂₇)(R₂₈);
10) —PO₃H₂;
11) —C(O)OR₂₇;
12) —C(O)N(R₂₇)(R₂₈);
13) —C(O)N(R₂₇)S(O)₂(R₂₈);
14) —N(R₂₇)(R₂₈);
15) —N(R₂₉)C(O)N(R₂₇)(R₂₈);
16) —N(R₂₇)C(O)(R₂₈);
17) —N(R₂₇)C(O)₂(R₂₈);
18) —N(R₂₉)S(O)₂N(R₂₇)(R₂₈);
19) —N(R₂₇)S(O)₂(R₂₈);
20) —OR₂₇; and
21) —OC(O)R₂₇; and
   Rₑ, L, Q1, Q2, Q3, Q4, S, T, U, V, R₂₇ and R₂₈ are as previously defined. In certain embodiments, the compound is represented by Formula (VI-1b) and at least three of Rₐ, R_{b}, R_{c}, and R_{d} are hydrogen. In certain embodiments, the compound is represented by Formula (VI-1b) and three of Rₐ, R_{b}, R_{c}, and R_{d} are hydrogen and the other is —C(O)OH. In certain embodiments, the compound is represented by Formula (VI-1b) where Rₐ, R_{b}, and R_{d} are hydrogen and R_{c} is —C(O)OH. In certain embodiments, the compound is represented by Formula (VI-1b) and three of Rₐ, R_{b}, R_{c}, and R_{d} are hydrogen or halogen and the other is —C(O)OH. In certain embodiments, the compound is represented by Formula (VI-1b) where Rₐ, R_{b}, and R_{d} are hydrogen or halogen and R_{c} is —C(O)OH.

In certain embodiments, the compound of Formula (I) is represented by one of Formulae (VII-1)˜ (VII-9),

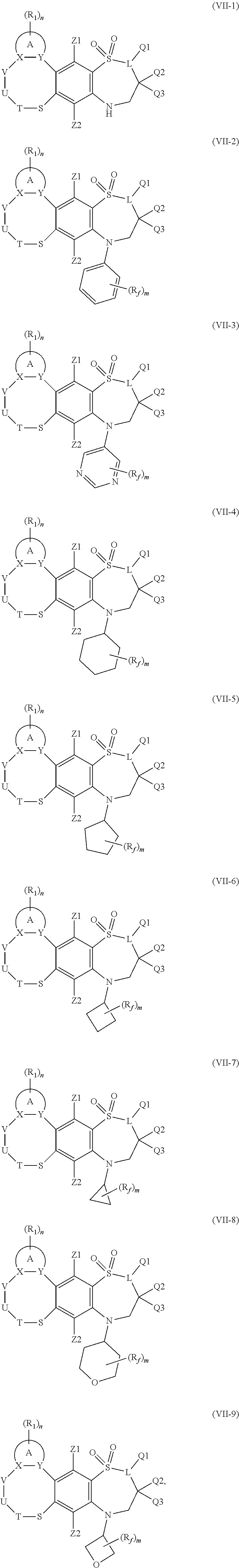

wherein each R_{f} is independently selected from:
1) halogen;
2) —CN;
3) —NO₂;
4) —OR₁₁;
5) —SR₁₁;
6) —NR₁₁R₁₂;
7) —OC(O)NR₁₁R₁₂;
8) optionally substituted —C₁-C₆ alkyl;
9) optionally substituted —C₃-C₈ cycloalkyl;
10) optionally substituted 3- to 8-membered heterocycloalkyl;
11) optionally substituted aryl; and
12) optionally substituted heteroaryl;
   m is 0, 1, 2, or 3; Q1, Q2, Q3, Z1, Z2, S, T, U, V, X, Y, A, n, R₁ R₁₁, and R₁₂ are as previously defined. In certain embodiments, m is 0 or 1. In certain embodiments m is 0. In certain embodiments, m is 1 and R_{f} is halogen, CN or optionally substituted C₁-C₆-alkyl.

In certain embodiments, the compound of Formula (I) is represented by one of Formulae (VIII-1)˜ (VIII-9),

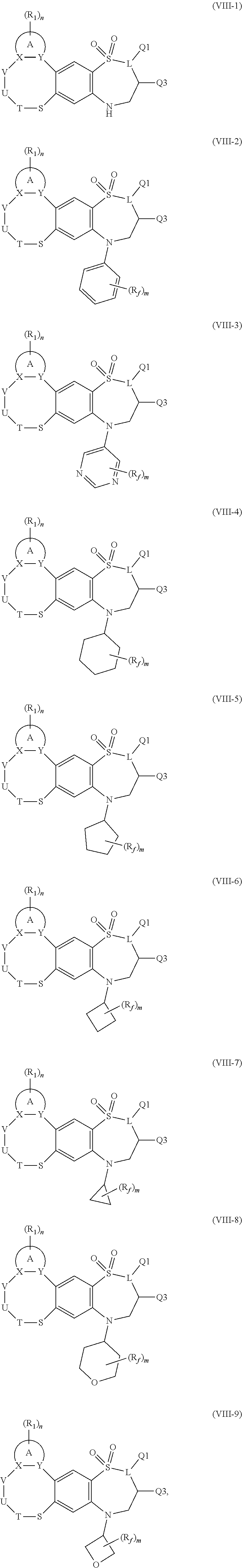

wherein m, Rf, Q1, Q3, S, T, U, V, X, Y, A, n and R₁ are previously defined.

In certain embodiments, the compound of Formula (I) is represented by one of Formulae (VIII-1a)˜ (VIII-9a),

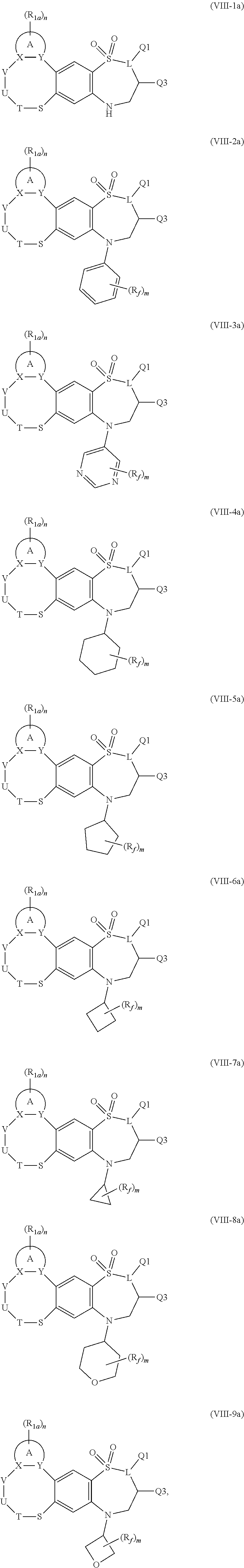

wherein m, R_{f}, Q1, Q3, S, T, U, V, X, Y, A, n and R₁ₐ are as previously defined.

In one embodiment, the compound of Formula (I) is represented by one of Formulae (IX-1)˜ (IX-23),

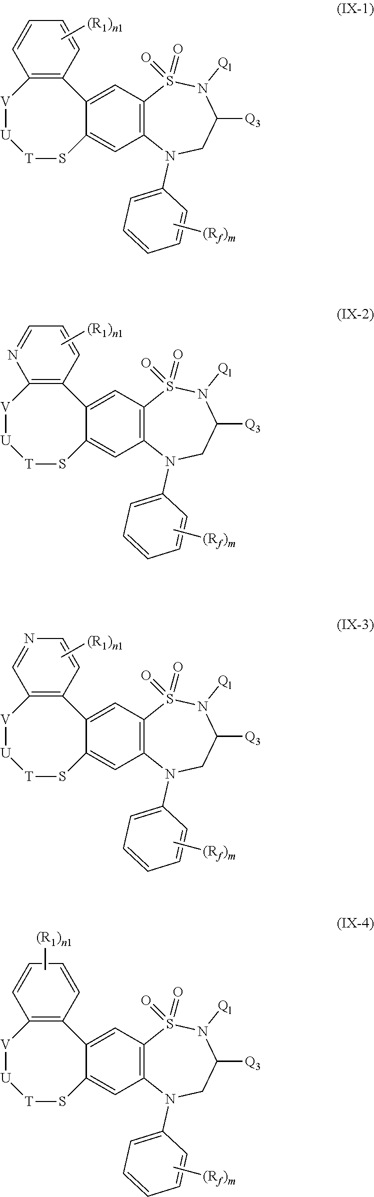

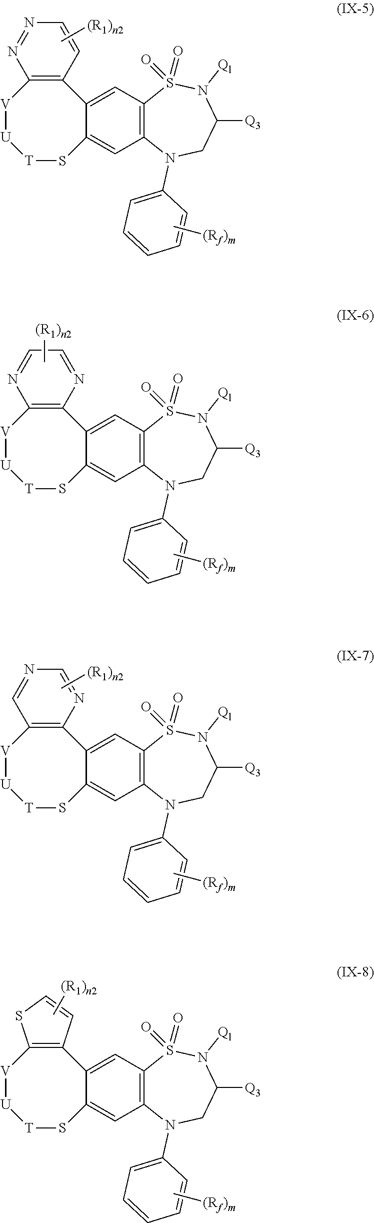

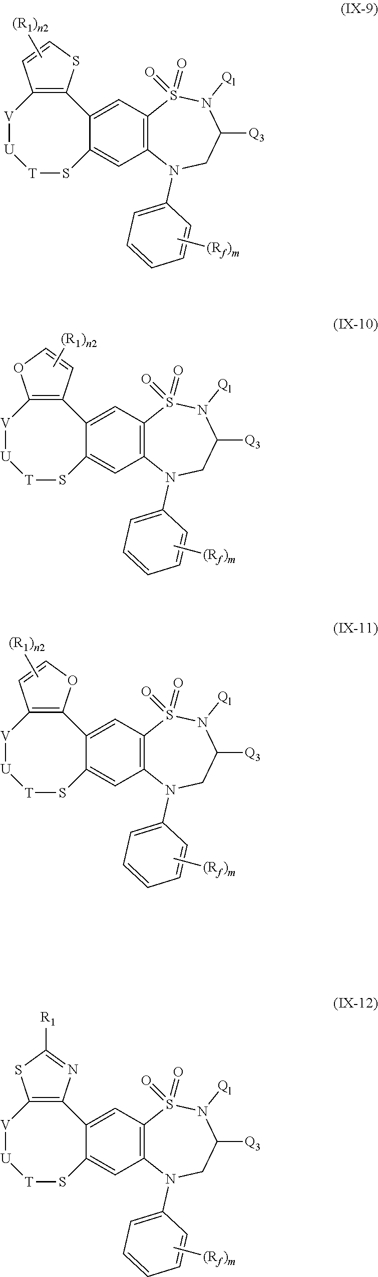

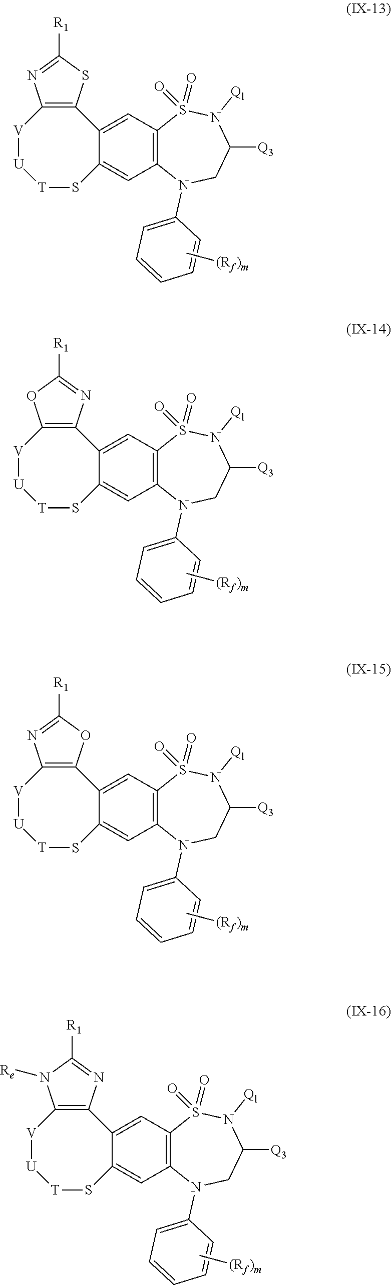

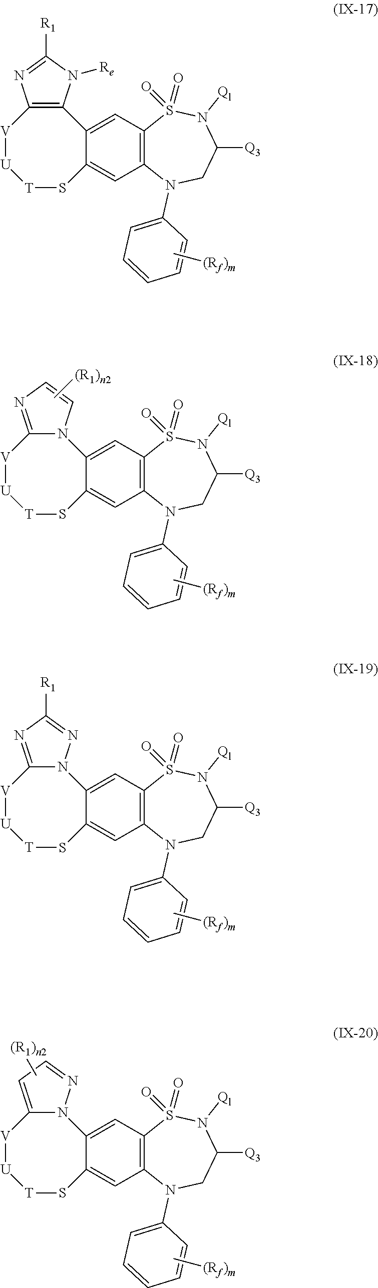

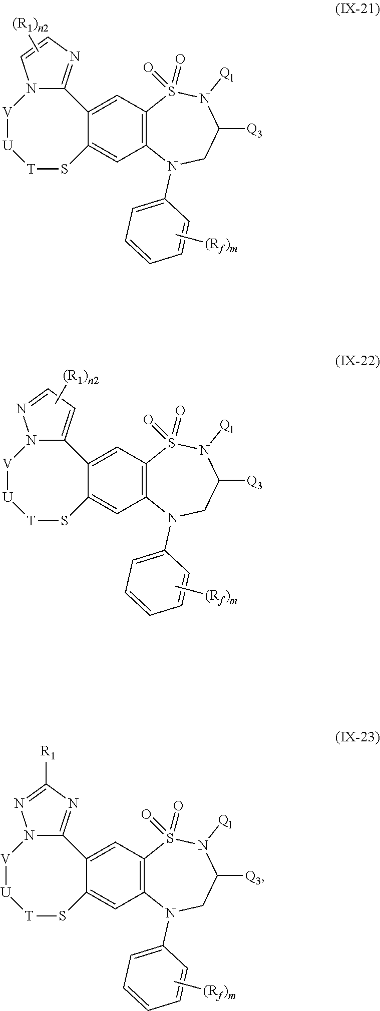

wherein n1, n2, m, R₁, Rₑ, R_{f}, Q1, Q3, S, T, U, and V are as previously defined.

In one embodiment of the present invention, the compound of Formula (I) is represented by one of Formulae (IX-1a)-(IX-23a),

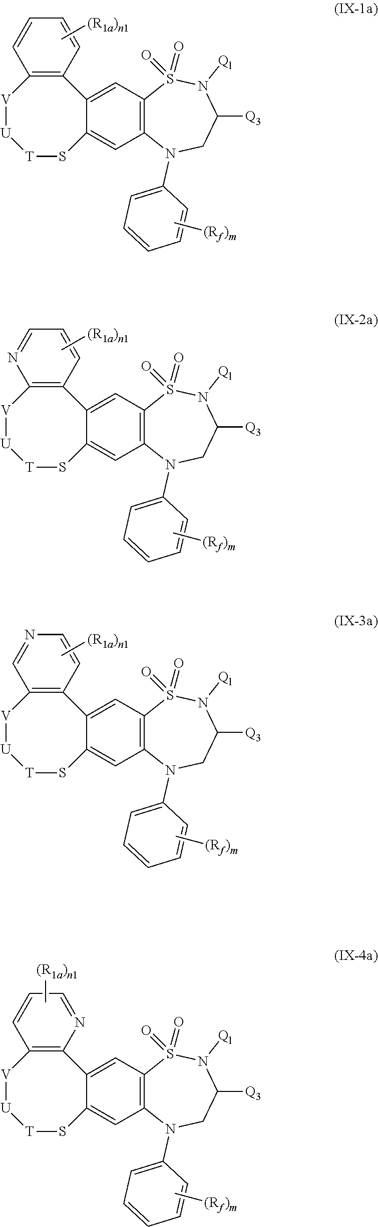

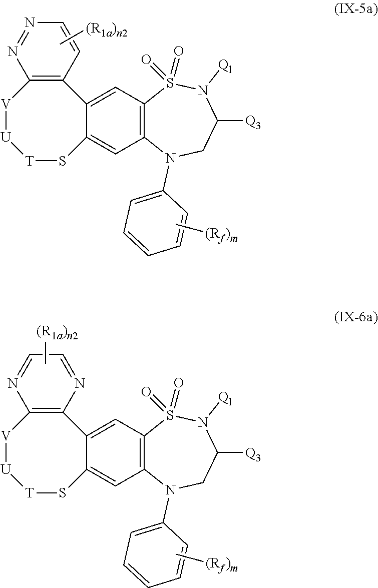

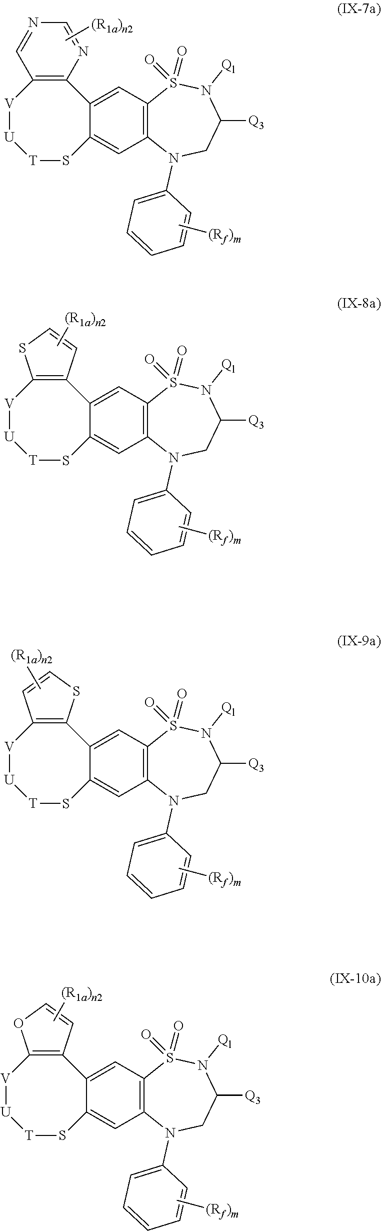

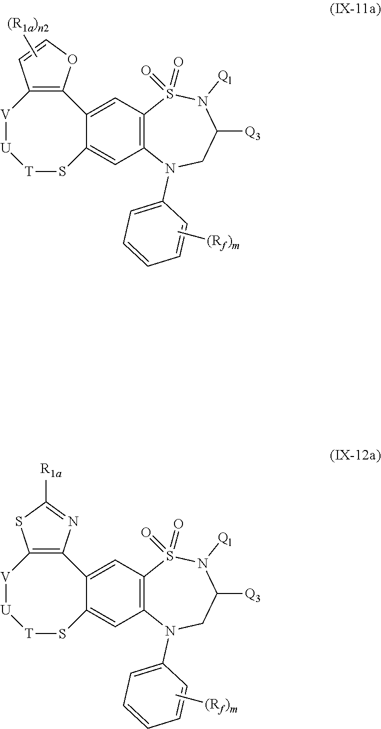

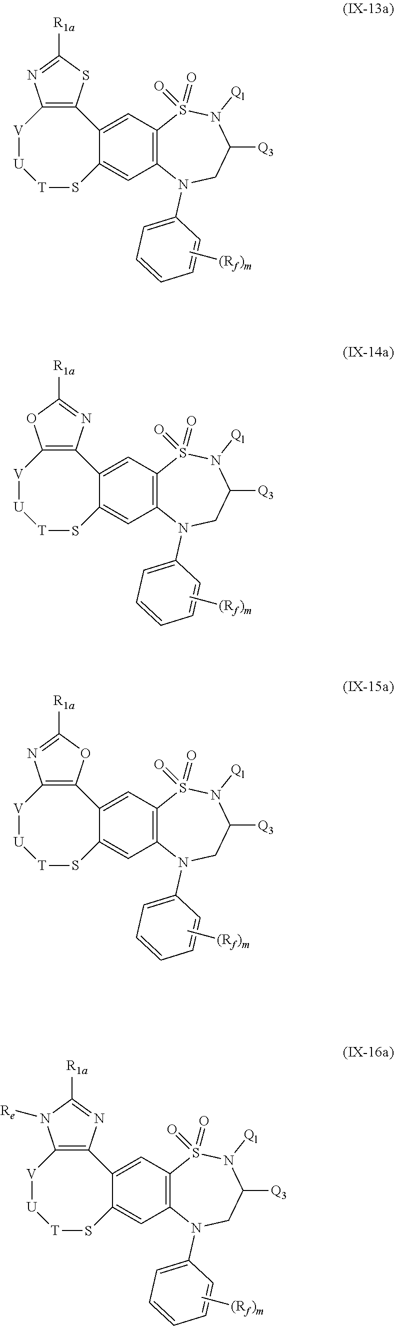

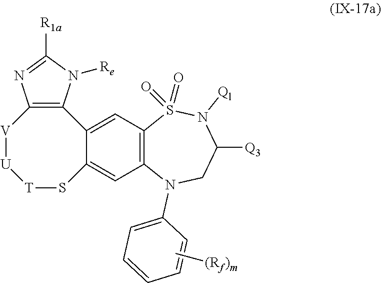

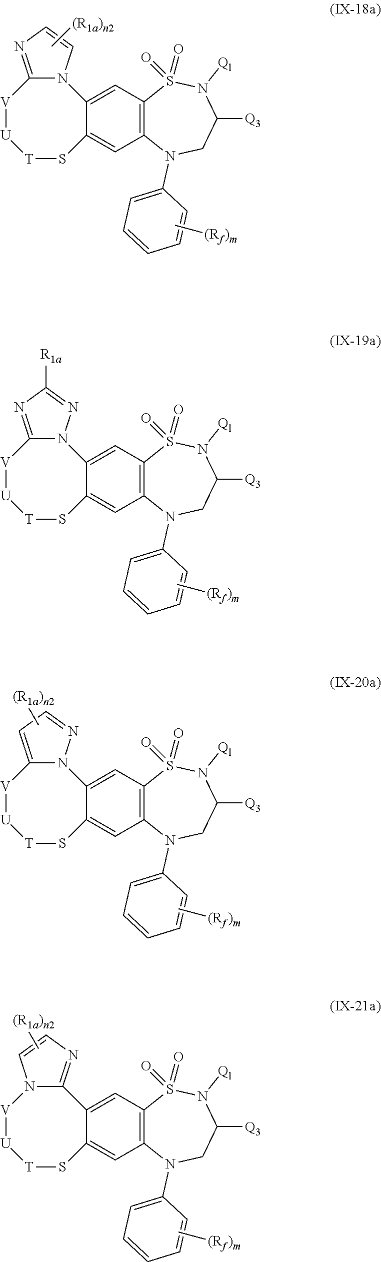

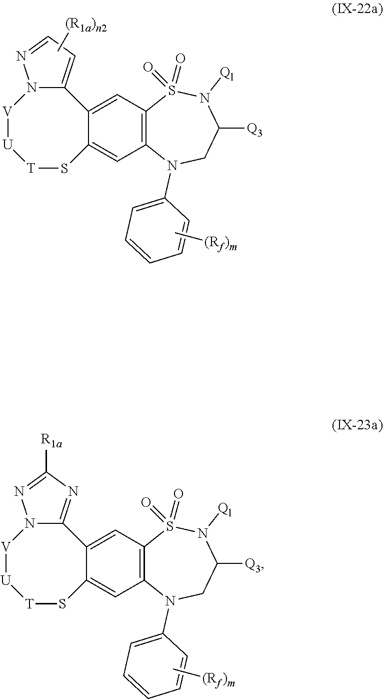

wherein n1, n2, m, R₁ₐ, Rₑ, R_{f}, Q1, Q3, S, T, U, and V are as previously defined.

In one embodiment of the present invention, the compound of Formula (I) is represented by one of Formulae (IX-1b)˜(IX-23b),

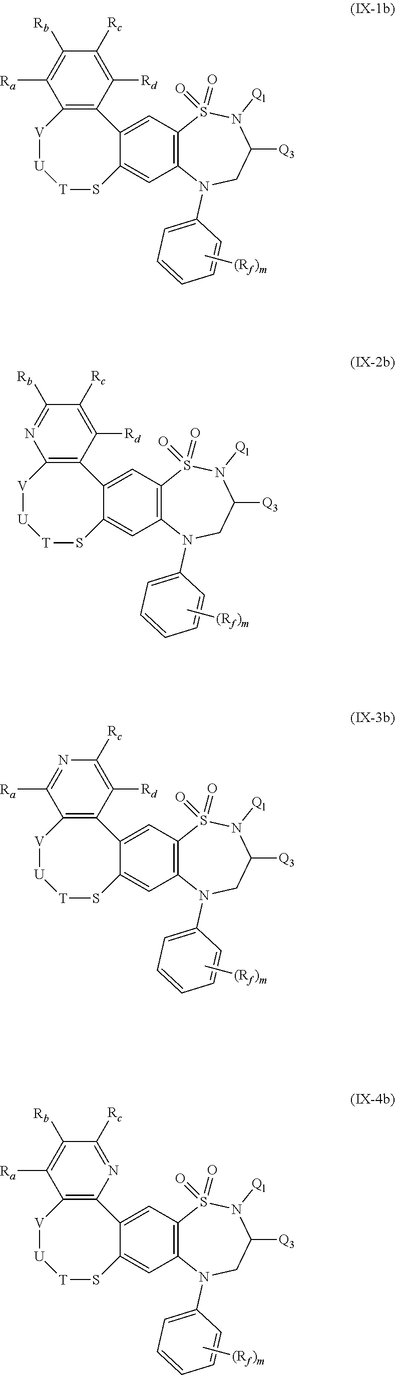

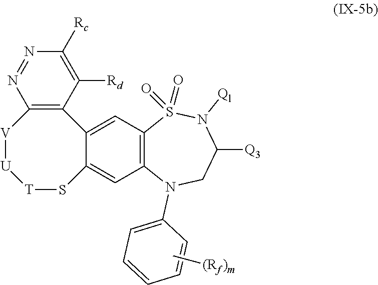

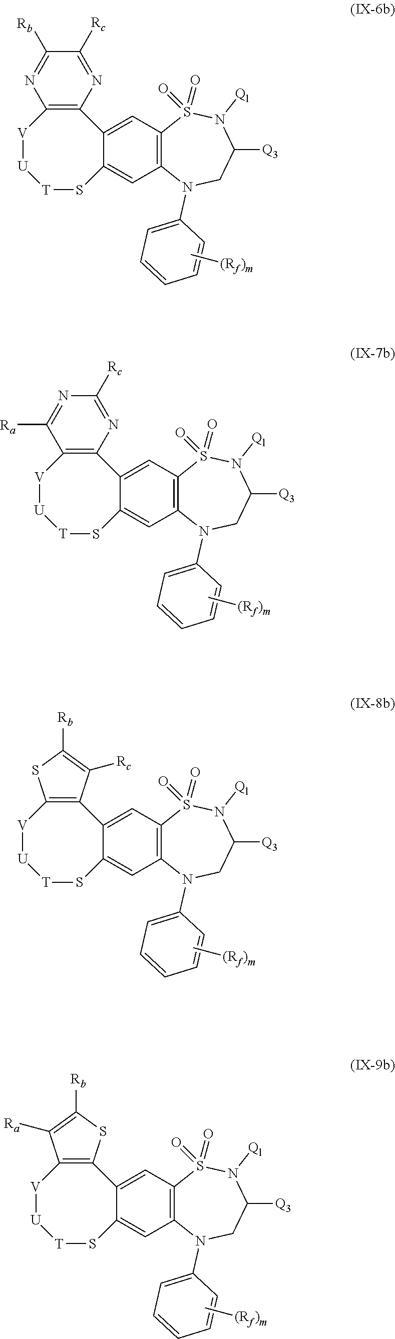

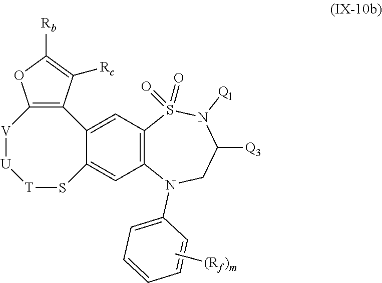

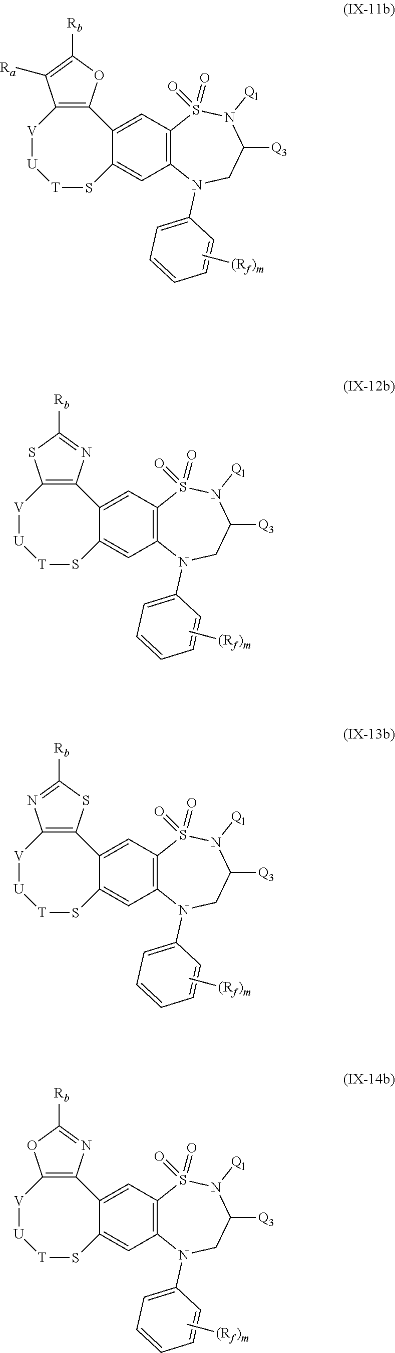

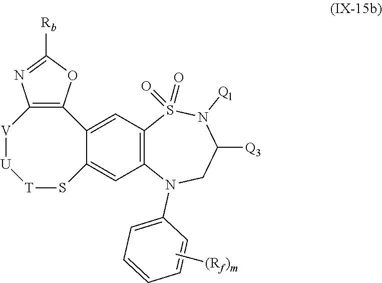

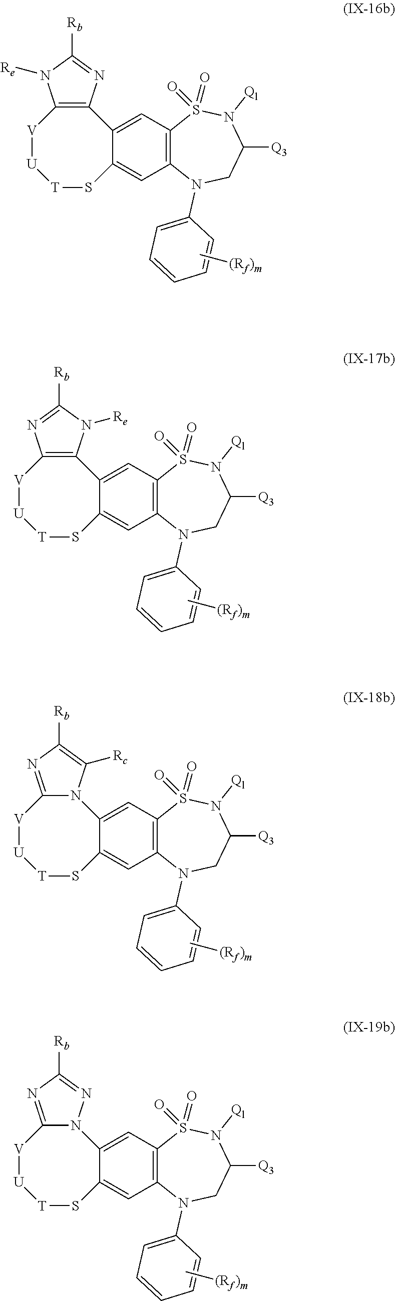

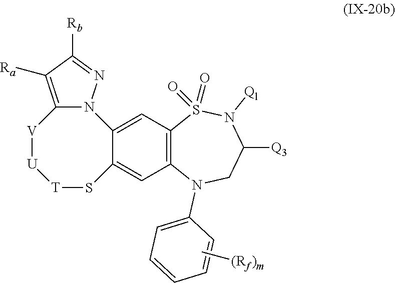

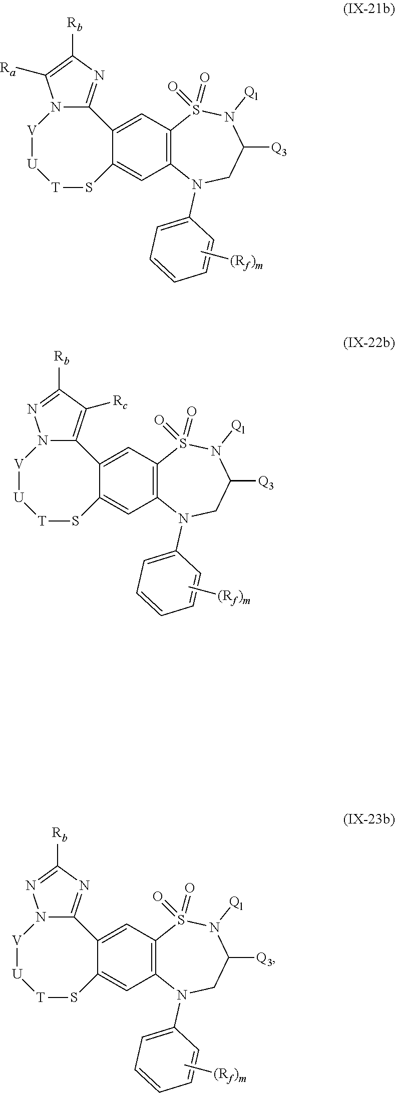

wherein Rₐ, R_{b}, R_{c}, R_{d}, Rₑ, m, R_{f}, Q1, Q3, S, T, U, and V are as previously defined. In certain embodiments, the compound is represented by Formula (IX-1b) and at least three of Rₐ, R_{b}, R_{c}, and R_{d} are hydrogen. In certain embodiments, the compound is represented by Formula (IX-1b) and three of Rₐ, R_{b}, R_{c}, and R_{d} are hydrogen and the other is —C(O)OH. In certain embodiments, the compound is represented by Formula (IX-1b) where Rₐ, R_{b}, and R_{d} are hydrogen and R_{c} is —C(O)OH. In certain embodiments, the compound is represented by Formula (IX-1b) and three of Rₐ, R_{b}, R_{c}, and R_{d} are independently hydrogen or halogen, preferably fluorine, and the other is —C(O)OH. In certain embodiments, the compound is represented by Formula (IX-1b) where Rₐ, R_{b}, and R_{d} are independently hydrogen or halogen, preferably fluorine, and R_{c} is —C(O)OH.

In one embodiment of the present invention, the compound of Formula (I) is represented by one of Formulae (IX-1c)˜ (IX-23c),

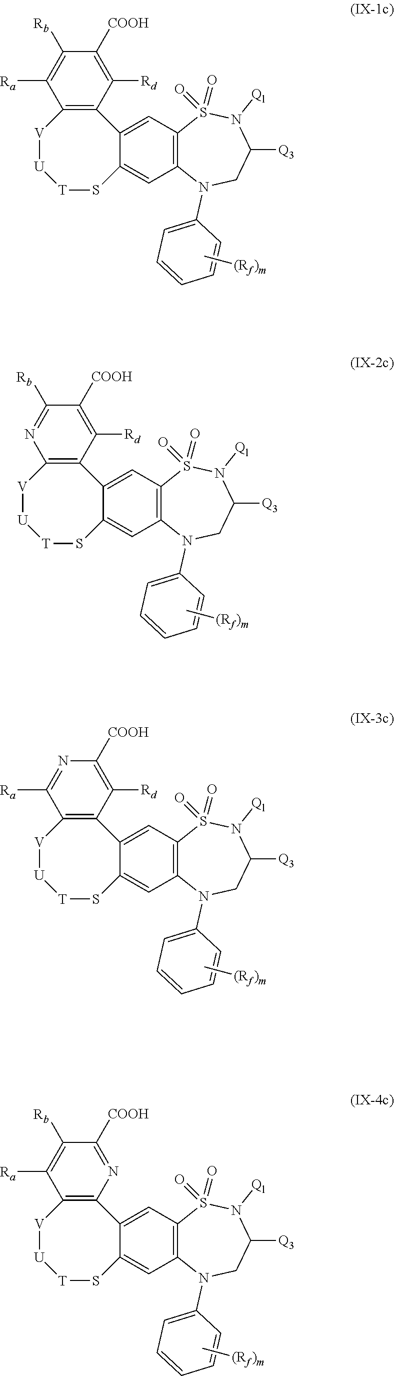

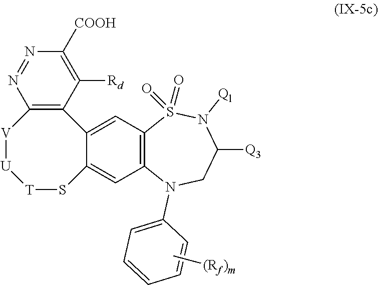

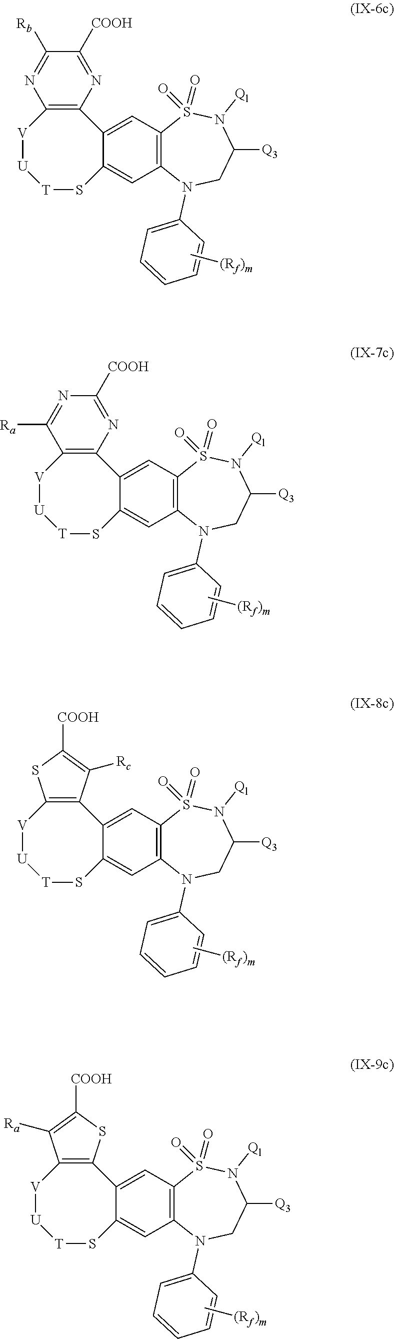

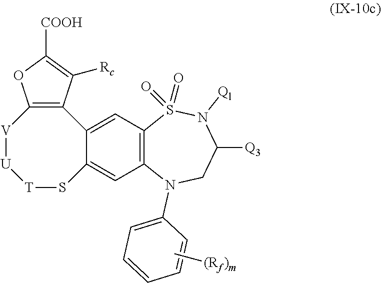

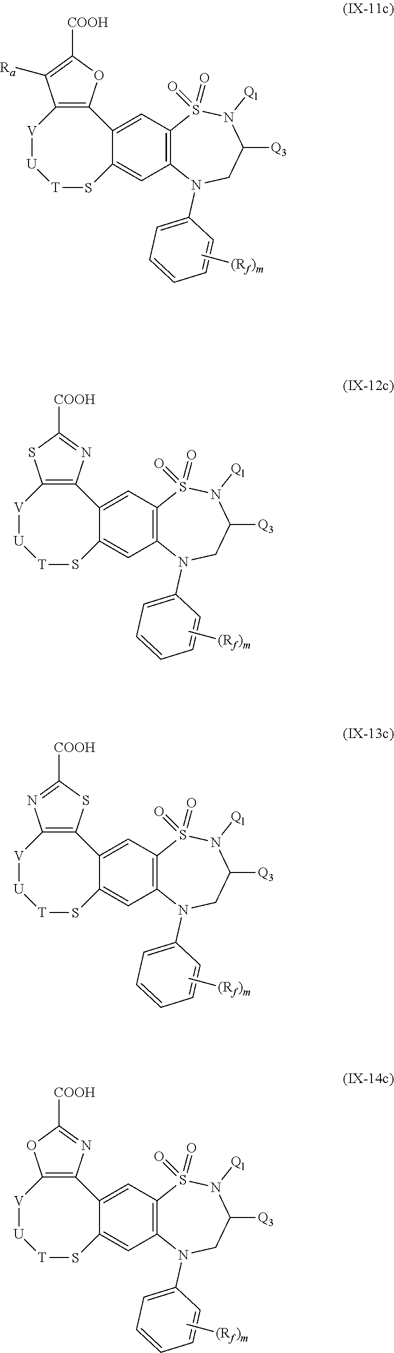

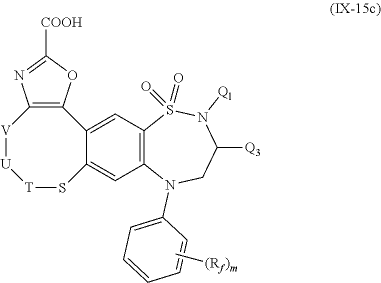

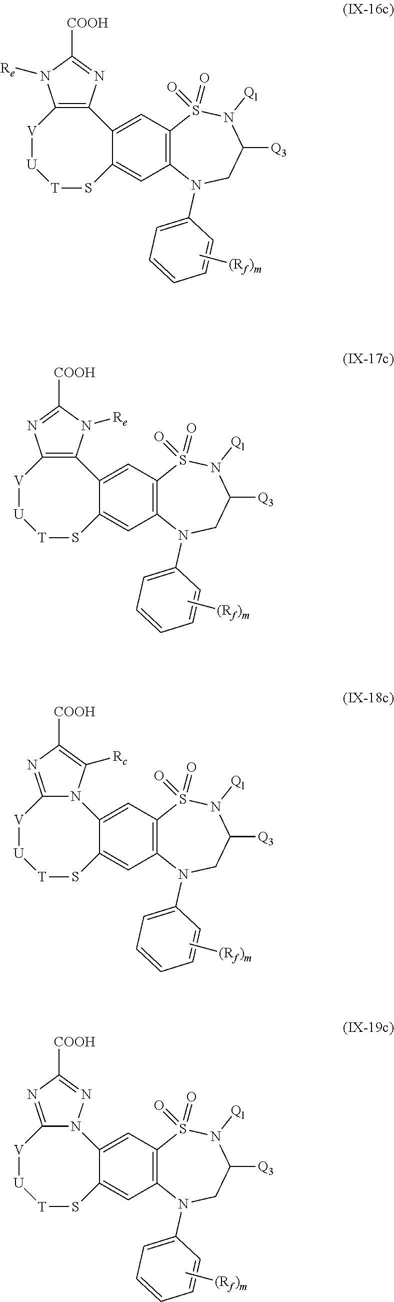

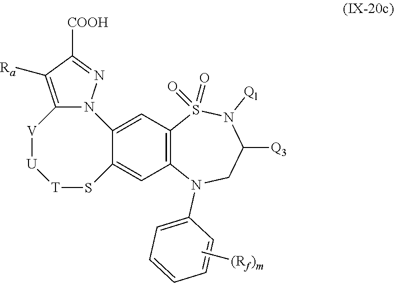

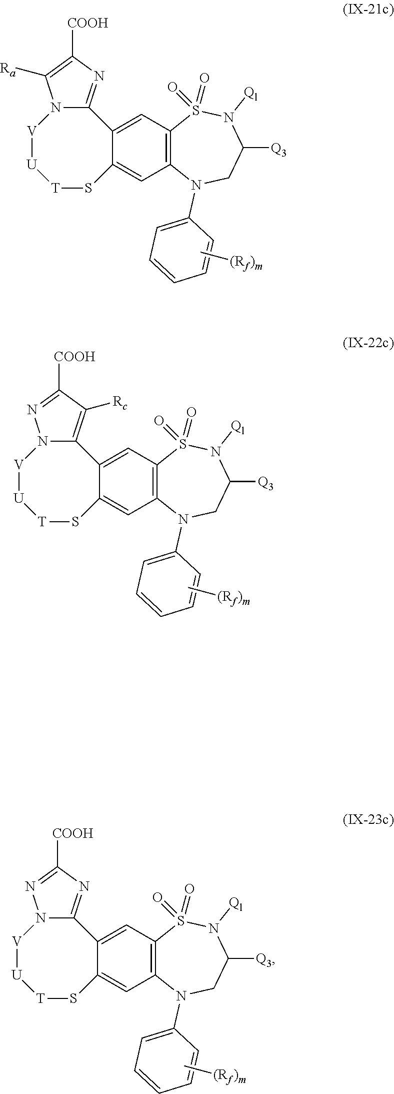

wherein Rₐ, R_{b}, R_{c}, R_{d}, Rₑ, m, R_{f}, Q1, Q3, S, T, U, and V are as previously defined.

In one embodiment of the present invention, the compound of Formula (I) is represented by one of Formulae (X-1)˜ (X-4),

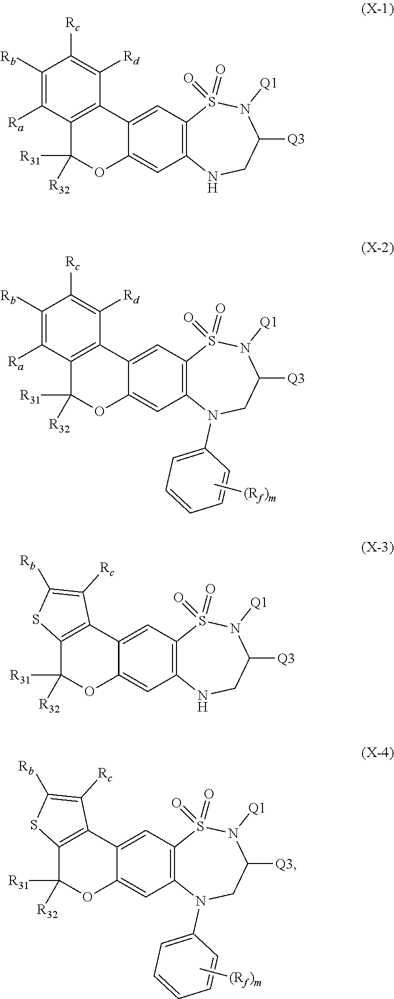

wherein R₃₁ and R₃₂ are independently selected from the group consisting of:
1) hydrogen;
2) halogen;
3) optionally substituted —C₁-C₆ alkyl;
4) optionally substituted —C₂-C₆ alkenyl
5) optionally substituted —C₃-C₈ cycloalkyl;
6) optionally substituted 3- to 8-membered heterocycloalkyl;
7) optionally substituted aryl; and
8) optionally substituted heteroaryl;
   alternatively, R₃₁ and R₃₂ are taken together with the carbon atom to which they are attached to form an optionally substituted 3- to 8-membered carbocyclic ring or an optionally substituted 3to 8-membered heterocyclic ring, and Rₐ, R_{b}, R_{c}, R_{d}, m, R_{f}, Q1, and Q3 are as previously defined. In certain embodiments, R₃₁ and R₃₂ are independently selected from hydrogen, halogen, C₁-C₄-alkyl and halo-C₁-C₄-alkyl. In certain embodiments, R₃₁ and R₃₂ are both hydrogen. In certain embodiments, R₃₁, R₃₂ and the carbon atom to which they are attached together form a C₃-C₆-cycloalkyl, preferably cyclopropyl. In certain embodiments, the compound is represented by Formula (X-1) and at least three of Rₐ, R_{b}, R_{c}, and R_{d} are hydrogen. In certain embodiments, the compound is represented by Formula (X-1) and three of Rₐ, R_{b}, R_{c}, and R_{d} are hydrogen and the other is —C(O)OH. In certain embodiments, the compound is represented by Formula (X-1) where Rₐ, R_{b}, and R_{d} are hydrogen and R_{c} is —C(O)OH.

In one embodiment of the present invention, the compound of Formula (I) is represented by one of Formulae (XI-1)˜ (XI-4),

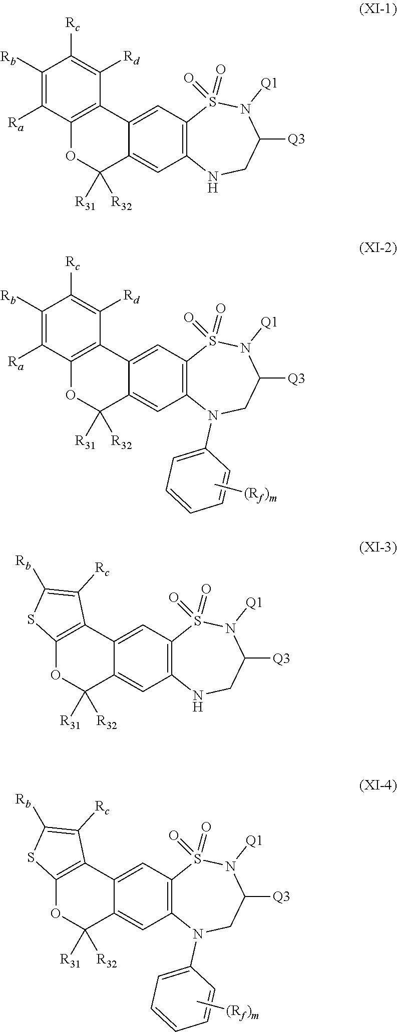

wherein R₃₁, R₃₂, Rₐ, R_{b}, R_{c}, R_{d}, m, R_{f}, Q1, and Q3 are as previously defined. In certain embodiments, the compound is represented by Formula (XI-1) or (XI-2) and at least three of Rₐ, R_{b}, R_{c}, and R_{d} are hydrogen. In certain embodiments, the compound is represented by Formula (XI-1) or (XI-2) and three of Rₐ, R_{b}, R_{c}, and R_{d} are hydrogen and the other is —C(O)OH. In certain embodiments, the compound is represented by Formula (XI-1) or (XI-2) where Rₐ, R_{b}, and R_{d} are hydrogen and R_{c} is —C(O)OH. In certain embodiments, the compound is represented by Formula (XI-1) or (XI-2) and three of Rₐ, R_{b}, R_{c}, and R_{d} are independently hydrogen or halogen, preferably fluorine, and the other is —C(O)OH. In certain embodiments, the compound is represented by Formula (XI-1) or (XI-2) where Rₐ, R_{b}, and R_{d} are independently hydrogen or halogen, preferably fluorine, and R_{c} is —C(O)OH.

In one embodiment of the present invention, the compound of Formula (I) is represented by one of Formulae (XII-1)˜ (XII-4),

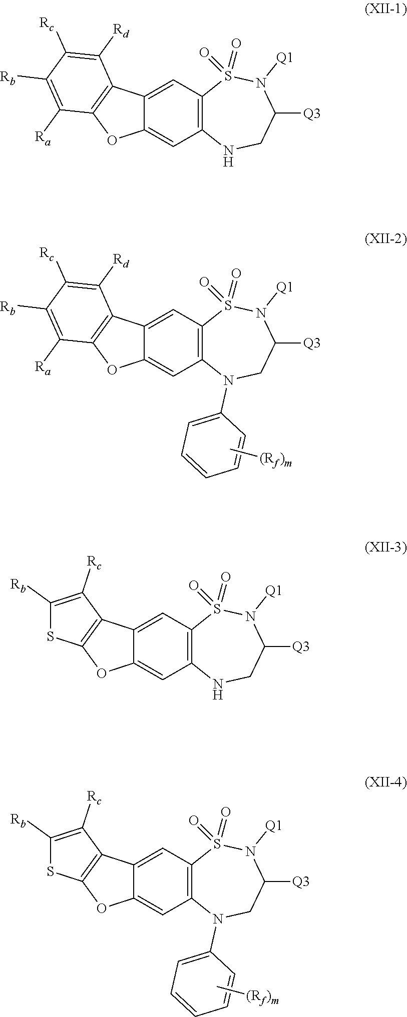

wherein Rₐ, R_{b}, R_{c}, R_{d}, m, R_{f}, Q1, and Q3 are as previously defined. In certain embodiments, the compound is represented by Formula (VI-1) and at least three of Rₐ, R_{b}, R_{c}, and R_{d} are hydrogen. In certain embodiments, the compound is represented by Formula (XII-1) or (XII-2) and three of Ra, R_{b}, R_{c}, and R_{d} are hydrogen and the other is —C(O)OH.

In one embodiment of the present invention, the compound of Formula (I) is represented by one of Formulae (XIII-1)˜ (XIII-4),

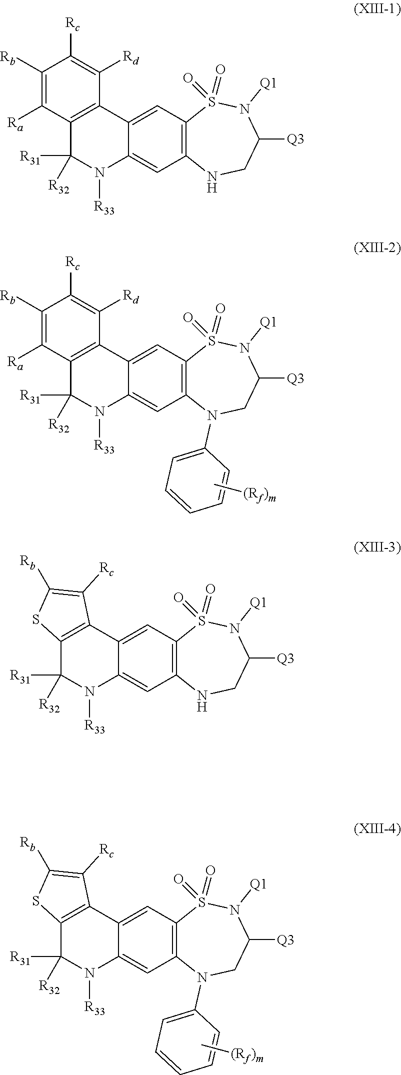

wherein R₃₃ is independently selected from the group consisting of:
1) hydrogen;
2) optionally substituted —C₁-C₆ alkyl;
3) optionally substituted —C₂-C₆ alkenyl
4) optionally substituted —C₃-C₈ cycloalkyl;
5) optionally substituted 3- to 8-membered heterocycloalkyl;
6) optionally substituted aryl; and
7) optionally substituted heteroaryl;
   and R₃₁, R₃₂, Ra, R_{b}, R_{c}, R_{d}, m, R_{f}, Q1, and Q3 are as previously defined. In certain embodiments, R₃₄ is selected from hydrogen, C₁-C₄-alkyl and optionally substituted phenyl. In certain embodiments, R₃₄ is selected from hydrogen and C₁-C₄-alkyl. In certain embodiments, the compound is represented by Formula (XIII-1) or (XIII-2) and at least three of Rₐ, R_{b}, R_{c}, and R_{d} are hydrogen. In certain embodiments, the compound is represented by Formula (XIII-1) or (XIII-2) and three of Rₐ, R_{b}, R_{c}, and R_{d} are hydrogen and the other is —C(O)OH. In certain embodiments, the compound is represented by Formula (XIII-1) or (XIII-2) where Rₐ, R_{b}, and R_{d} are hydrogen and R_{c} is —C(O)OH. In certain embodiments, the compound is represented by Formula (XIII-1) or (XIII-2) and three of Rₐ, R_{b}, R_{c}, and R_{d} are independently hydrogen or halogen, preferably fluorine, and the other is —C(O)OH. In certain embodiments, the compound is represented by Formula (XIII-1) or (XIII-2) where Rₐ, R_{b}, and R_{d} are independently hydrogen or halogen, preferably fluorine, and R_{c} is —C(O)OH.

In one embodiment of the present invention, the compound of Formula (I) is represented by one of Formulae (XIV-1)˜ (XIV-4),

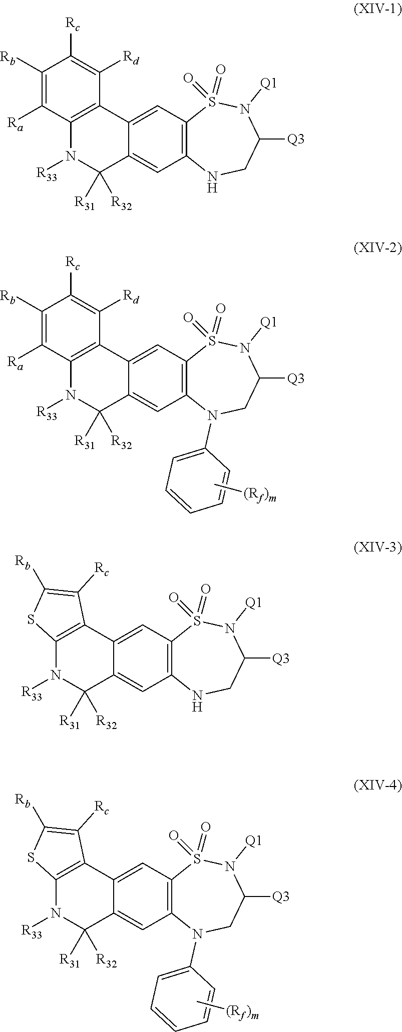

wherein R₃₁, R₃₂, R₃₃, Rₐ, R_{b}, R_{c}, R_{d}, m, R_{f}, Q1, and Q3 are as previously defined. In certain embodiments, the compound is represented by Formula (XIV-1) or (XIV-2) and at least three of Rₐ, R_{b}, R_{c}, and R_{d} are hydrogen. In certain embodiments, the compound is represented by Formula (XIV-1) or (XIV-2) and three of Rₐ, R_{b}, R_{c}, and R_{d} are hydrogen and the other is —C(O)OH. In certain embodiments, the compound is represented by Formula (XIV-1) or (XIV-2) where Rₐ, R_{b}, and R_{d} are hydrogen and R_{c} is —C(O)OH. In certain embodiments, the compound is represented by Formula (XIV-1) or (XIV-2) and three of Rₐ, R_{b}, R_{c}, and R_{d} are independently hydrogen or halogen, preferably fluorine, and the other is —C(O)OH. In certain embodiments, the compound is represented by Formula (XIV-1) or (XIV-2) where Rₐ, R_{b}, and R_{d} are independently hydrogen or halogen, preferably fluorine, and R_{c} is —C(O)OH.

In one embodiment of the present invention, the compound of Formula (I) is represented by one of Formulae (X-1a)˜ (X-4a),

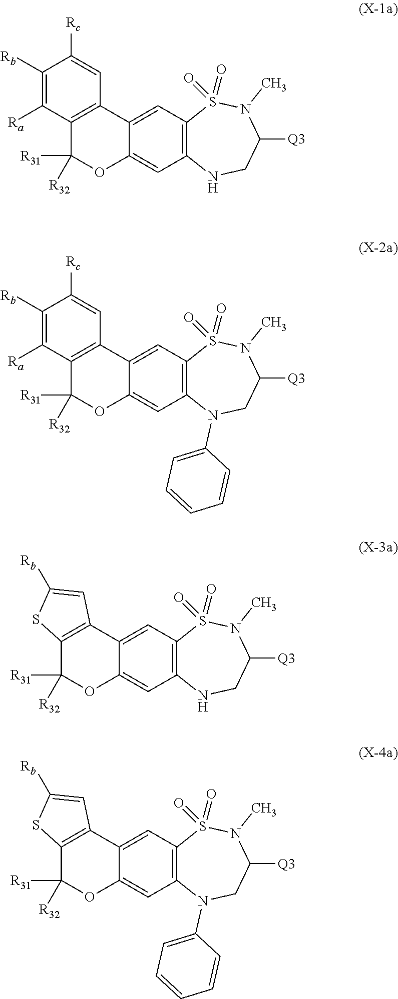

wherein R₃₁, R₃₂, Rₐ, R_{b}, R_{c}, and Q3 are as previously defined. In certain embodiments, the compound is represented by Formula (X-1a) or (X-2a) and at least three of Rₐ, R_{b}, R_{c}, and R_{d} are hydrogen. In certain embodiments, the compound is represented by Formula (X-1a) or (X-2a) and three of Rₐ, R_{b}, R_{c}, and R_{d} are hydrogen and the other is —C(O)OH. In certain embodiments, the compound is represented by Formula (X-1a) or (X-2a) where Rₐ, R_{b}, and R_{d} are hydrogen and R_{c} is —C(O)OH. In certain embodiments, the compound is represented by Formula (X-1a) or (X-2a) and three of Rₐ, R_{b}, R_{c}, and R_{d} are independently hydrogen or halogen, preferably fluorine, and the other is —C(O)OH. In certain embodiments, the compound is represented by Formula (X-1a) or (X-2a) where Rₐ, R_{b}, and R_{d} are independently hydrogen or halogen, preferably fluorine, and R_{c} is —C(O)OH.

In one embodiment of the present invention, the compound of Formula (I) is represented by one of Formulae (XIII-1a)˜ (XIII-4a),

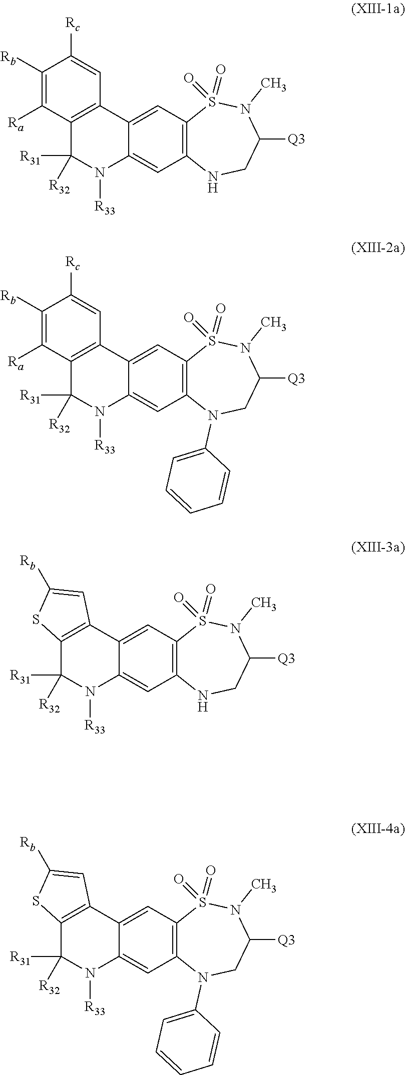

wherein R₃₁, R₃₂, R₃₃, Rₐ, R_{b}, R_{c}, and Q3 are as previously defined. In certain embodiments, the compound is represented by Formula (XIII-1a) or (XIII-2a) and at least two of Rₐ, R_{b}, and R_{c} are hydrogen. In certain embodiments, the compound is represented by Formula (XIII-1a) or (XIII-2a) and two of Rₐ, R_{b}, and R_{c} are hydrogen and the other is —C(O)OH. In certain embodiments, the compound is represented by Formula (XIIII-1a) or (XIII-2a) where Rₐ and R_{b} are hydrogen and R_{c} is —C(O)OH. In certain embodiments, the compound is represented by Formula (XIII-1a) or (XIII-2a) and two of Rₐ, R_{b}, and R_{c} are independently hydrogen or halogen, preferably fluorine, and the other is —C(O)OH. In certain embodiments, the compound is represented by Formula (XIIII-1a) or (XIII-2a) where Rₐ and R_{b} are independently hydrogen or halogen, preferably fluorine, and R_{c} is —C(O)OH.

In one embodiment of the present invention, the compound of Formula (I) is represented by one of Formulae (XV-1)˜ (XV-10),

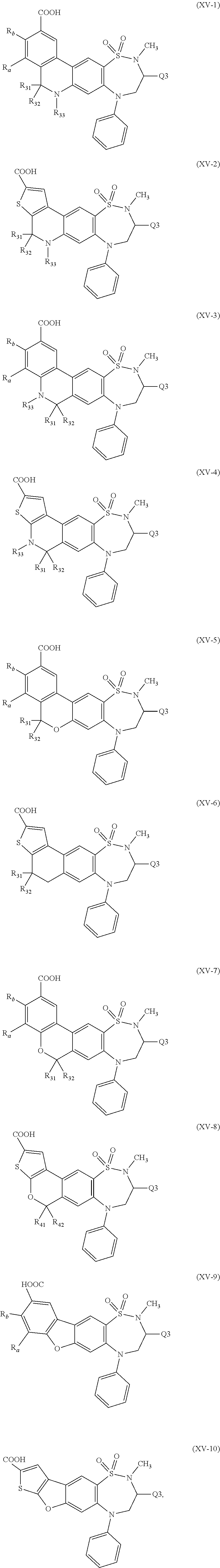

wherein R₃₁, R₃₂, R₃₃, Rₐ, R_{b}, and Q3 are as previously defined.

It will be appreciated that the description of the present invention herein should be construed in congruity with the laws and principles of chemical bonding. In some instances, it may be necessary to remove a hydrogen atom in order to accommodate a substituent at any given location.

It will be yet appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be achieved by concomitant administration in: (1) a unitary pharmaceutical composition including both compounds; or (2) separate pharmaceutical compositions each including one of the compounds.

In certain embodiments of the combination therapy, the additional therapeutic agent is administered at a lower dose and/or dosing frequency as compared to dose and/or dosing frequency of the additional therapeutic agent required to achieve similar results in treating or preventing hepatitis B virus (HBV) and/or hepatitis D virus (HDV).

It should be understood that the compounds encompassed by the present invention are those that are suitably stable for use as a pharmaceutical agent.

In still another embodiment of the method, administering the compound of the invention reduces viral load in the individual to a greater extent compared to the administering of a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

In another embodiment, administering of the compound of the invention causes a lower incidence of viral mutation and/or viral resistance than the administering of a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

It should be understood that the compounds encompassed by the present invention are those that are suitably stable for use as pharmaceutical agent.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8t, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₅ cycloalkenyl and C₅-C₇ cycloalkenyl groups.

Examples of cycloalkenyl include, but are not limited to, cyclopropenyl, cyclobutenyl, cyclo pentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups includes, but are not limited to, methoxy, ethoxy, n-propoxy, 2-propoxy (isopropoxy) and the higher homologs and isomers. Preferred alkoxy is C₁-C₃alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)— heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH— heterocycloalkyl, —OCO₂-C₁-C₁₂-alkyl, —OCO₂-C₂-C₈-alkenyl, —OCO₂-C₂-C₈-alkynyl, —OCO₂-C₃-C₁₂-cycloalkyl, —OCO₂-aryl, —OCO₂-heteroaryl, —OCO₂-heterocycloalkyl, —CO₂-C₁-C₁₂ alkyl, —CO₂-C₂-C₈ alkenyl, —CO₂-C₂-C₈ alkynyl, —CO₂-C₃-C₁₂-cycloalkyl, —CO₂-aryl, —CO₂-heteroaryl, —CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH— heterocycloalkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)— heterocycloalkyl, —NHCO₂—C₁-C₁₂-alkyl, —NHCO₂-C₂-C₈-alkenyl, —NHCO₂-C₂-C₈-alkynyl, —NHCO₂-C₃-C₁₂-cycloalkyl, —NHCO₂-aryl, —NHCO₂-heteroaryl, —NHCO₂— heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH— heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH₂, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)— heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂-C₁-C₁₂-alkyl, —SO₂-C₂-C₈-alkenyl, —SO₂-C₂-C₈-alkynyl, —SO₂-C₃-C₁₂-cycloalkyl, —SO₂-aryl, —SO₂-heteroaryl, —SO₂-heterocycloalkyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂-C₂-C₈-alkenyl, —NHSO₂-C₂-C₈-alkynyl, —NHSO₂-C₃-C₁₂-cycloalkyl, —NHSO₂-aryl, —NHSO₂— heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably C₁ and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); P. G. M. Wuts, Greene's Protective Groups in Organic Synthesis, 5th edition, John Wiley & Sons, Hoboken, N J (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Combination and Alternation Therapy

In one embodiment, the compounds described herein are suitable for monotherapy and are effective against natural or native HBV and/or HDV strains and against HBV and/or HDV strains resistant to currently known drugs. In another embodiment, the compounds described herein are suitable for use in combination therapy.

In another embodiment, the additional therapeutic agent is selected from a core inhibitor, which includes GLS4, GLS4JHS, JNJ-379, ABI-H₀₇₃₁, ABI-H₂₁₅₈, AB-423, AB-506, WX-066, and QL-OA6A; immune modulator or immune stimulator therapies, which includes T-cell response activator AIC649 and biological agents belonging to the interferon class, such as interferon alpha 2a or 2b or modified interferons such as pegylated interferon, alpha 2a, alpha 2b, lamda; or STING (stimulator of interferon genes) modulator; or TLR modulators such as TLR-7 agonists, TLR-8 agonists or TLR-9 agonists; or therapeutic vaccines to stimulate an HBV-specific immune response such as virus-like particles composed of HBcAg and HBsAg, immune complexes of HBsAg and HBsAb, or recombinant proteins comprising HBx, HBsAg and HBcAg in the context of a yeast vector; or immunity activator such as SB-9200 of certain cellular viral RNA sensors such as RIG-I, NOD2, and MDA5 protein, or RNA interence (RNAi) or small interfering RNA (siRNA) such as ARC-520, ARC-521, ARB-1467, and ALN-HBV RNAi, or antiviral agents that block viral entry or maturation or target the HBV polymerase such as nucleoside or nucleotide or non-nucleos(t)ide polymerase inhibitors, and agents of distinct or unknown mechanism including agents that disrupt the function of other essential viral protein(s) or host proteins required for HBV replication or persistence such as REP 2139, RG7834, and AB-452. In an embodiment of the combination therapy, the reverse transcriptase inhibitor is at least one of Zidovudine, Didanosine, Zalcitabine, ddA, Stavudine, Lamivudine, Aba-cavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In another embodiment of the combination therapy, the TLR-7 agonist is selected from the group consisting of SM360320 (12-benzyl-8-hydroxy-2-(2-methoxy-ethoxy)ad-enine), AZD 8848 (methyl[3-({[3-(6-amino-2-butoxy-8-oxo-7,8-dihydro-9H-purin-12-yl)propyl][3-(4-morpholinyl) propyl] amino Imethyl)phenyl] acetate), GS-9620 (4-Amino-2-butoxy-8-[3-(2-pyrrolidinylmethyl)benzyl]-7,8-dihydro-6(5H)-pteridinone), AL-034 (TQ-A3334), and RO6864018.

In another embodiment of the combination therapy, the TLR-8 agonist is GS-9688.

In an embodiment of these combination therapies, the compound and the additional therapeutic agent are co-formulated. In another embodiment, the compound and the additional therapeutic agent are co-administered.

In another embodiment of the combination therapy, administering the compound of the invention allows for administering of the additional therapeutic agent at a lower dose or frequency as compared to the administering of the at least one additional therapeutic agent alone that is required to achieve similar results in prophylactically treating an HBV infection in an individual in need thereof.

In another embodiment of the combination therapy, before administering the therapeutically effective amount of the compound of the invention, the individual is known to be refractory to a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

Preferred compounds for combination or alternation therapy for the treatment of HBV include 3TC, FTC, L-FMAU, interferon, adefovir dipivoxil, entecavir, telbivudine (L-dT), valtorcitabine (3′-valinyl L-dC), β-D-dioxolanyl-guanine (DXG), β-D-dioxolanyl-2,6-diaminopurine (DAPD), and β-D-dioxolanyl-6-chloropurine (ACP), famciclovir, penciclovir, lobucavir, ganciclovir, and ribavirin.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The “additional therapeutic or prophylactic agents” include but are not limited to, immune therapies (e.g., interferon), therapeutic vaccines, antifibrotic agents, anti-inflammatory agents such as corticosteroids or NSAIDs, bronchodilators such as beta-2 adrenergic agonists and xanthines (e.g., theophylline), mucolytic agents, anti-muscarinics, anti-leukotrienes, inhibitors of cell adhesion (e.g., ICAM antagonists), anti-oxidants (e.g., N-acetylcysteine), cytokine agonists, cytokine antagonists, lung surfactants and/or antimicrobial and anti-viral agents (e.g., ribavirin and amantadine). The compositions according to the invention may also be used in combination with gene replacement therapy.

Although the invention has been described with respect to various preferred embodiments, it is not intended to be limited thereto, but rather those skilled in the art will recognize that variations and modifications may be made therein which are within the spirit of the invention and the scope of the appended claims.

### Antiviral Activity

An inhibitory amount or dose of the compounds of the present invention may range from about 0.01 mg/Kg to about 500 mg/Kg, alternatively from about 1 to about 50 mg/Kg. Inhibitory amounts or doses will also vary depending on route of administration, as well as the possibility of co-usage with other agents.

According to the methods of treatment of the present invention, viral infections are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### Abbreviations

Abbreviations which may be used in the descriptions of the scheme and the examples that follow are: Ac for acetyl; AcOH for acetic acid; Boc₂O for di-tert-butyl-dicarbonate; Boc for t-butoxycarbonyl; Bz for benzoyl; Bn for benzyl; t-BuOK for potassium tert-butoxide; Brine for sodium chloride solution in water; CDI for carbonyldiimidazole; DCM or CH₂Cl₂ for dichloromethane; CH₃ for methyl; CH₃CN for acetonitrile; Cs₂CO₃ for cesium carbonate; CuCl for copper (I) chloride; CuI for copper (I) iodide; dba for dibenzylidene acetone; DBU for 1,8-diazabicyclo[5.4.0]-undec-7-ene; DEAD for diethylazodicarboxylate; DIAD for diisopropyl azodicarboxylate; DIPEA or (i-Pr)₂EtN for N,N,-diisopropylethyl amine; DMP or Dess-Martin periodinane for 1,1,2-tris(acetyloxy)-1,2-dihydro-1,2-benziodoxol-3-(1H)-one; DMAP for 4-dimethylamino-pyridine; DME for 1,2-dimethoxyethane; DMF for N,N-dimethylformamide; DMSO for dimethyl sulfoxide; EtOAc for ethyl acetate; EtOH for ethanol; Et₂O for diethyl ether; HATU for O-(7-azabenzotriazol-2-yl)-N,N,N′,N′,-tetramethyluronium Hexafluoro-phosphate; HCl for hydrogen chloride; K₂CO₃ for potassium carbonate; n-BuLi for n-butyl lithium; DDQ for 2,3-dichloro-5,6-dicyano-1,4-benzoquinone; LDA for lithium diisopropylamide; LiTMP for lithium 2,2,6,6-tetramethyl-piperidinate; MeOH for methanol; Mg for magnesium; MOM for methoxymethyl; Ms for mesyl or —SO₂—CH₃; NaHMDS for sodium bis(trimethylsilyl)amide; NaCl for sodium chloride; NaH for sodium hydride; NaHCO₃ for sodium bicarbonate or sodium hydrogen carbonate; Na₂CO₃ sodium carbonate; NaOH for sodium hydroxide; Na₂SO₄ for sodium sulfate; NaHSO₃ for sodium bisulfite or sodium hydrogen sulfite; Na₂S₂O₃ for sodium thiosulfate; NH₂NH₂ for hydrazine; NH₄Cl for ammonium chloride; Ni for nickel; OH for hydroxyl; OsO₄ for osmium tetroxide; OTf for triflate; PPA for polyphophoric acid; PTSA for p-toluenesulfonic acid; PPTS for pyridinium p-toluenesulfonate; TBAF for tetrabutylammonium fluoride; TEA or Et₃N for triethylamine; TES for triethylsilyl; TESCl for triethylsilyl chloride; TESOTf for triethylsilyl trifluoromethanesulfonate; TFA for trifluoroacetic acid; THF for tetrahydrofuran; TMEDA for N,N,N′,N′-tetramethylethylene-diamine; TPP or PPh₃ for triphenyl-phosphine; Tos or Ts for tosyl or —SO₂-C₆H₄CH₃; Ts₂O for tolylsulfonic anhydride or tosyl-anhydride; TsOH for p-tolylsulfonic acid; Pd for palladium; Ph for phenyl; Pd₂(dba)₃ for tris(diben-zylideneacetone) dipalladium (0); Pd(PPh₃)₄ for tetrakis(triphenylphosphine)-palladium (0); PdCl₂(PPh₃)₂ for trans-dichlorobis-(triphenylphosphine)palladium (II); Pt for platinum; Rh for rhodium; rt for room temperature; Ru for ruthenium; TBS for tert-butyl dimethylsilyl; TMS for trimethylsilyl; or TMSCl for trimethylsilyl chloride.

### Synthetic Methods

The compounds and processes of the present invention will be better understood in connection with the following synthetic schemes that illustrate the methods by which the compounds of the invention may be prepared. These schemes are of illustrative purpose and are not meant to limit the scope of the invention. Equivalent, similar, or suitable solvents, reagents or reaction conditions may be substituted for those particular solvents, reagents, or reaction conditions described herein without departing from the general scope of the method of synthesis.

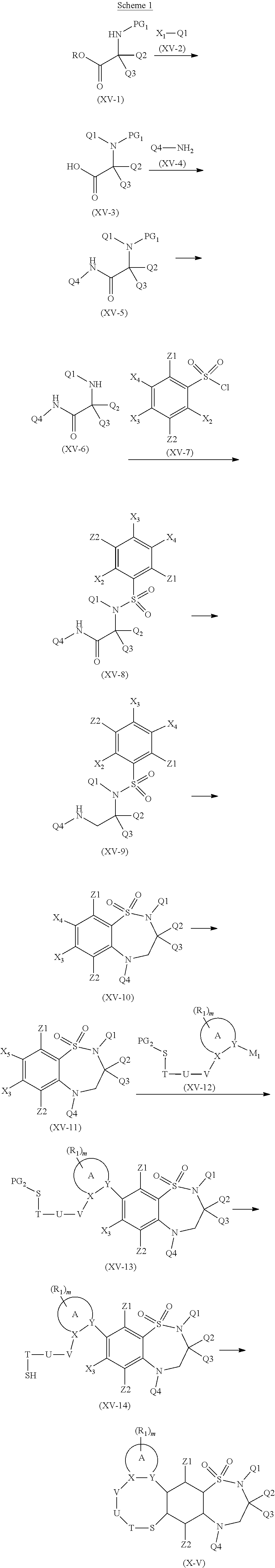

Scheme 1 illustrates a general method to prepare the compound of formula X-V, wherein Q1, Q2, Q3, Q4, Z1, Z2, S, T, U, V, X, Y, A, and R₁ are as previously defined, from the amino acid (R═H) or amino ester (R=alkyl) derivative XV-1. The PG₁ is a common amino acid protecting group including but not limited to Cbz, Boc, or Fmoc. When Q1 is not H, R=alkyl, and Q₁ can be installed via displacement of the leaving group X₁ (Cl, Br, I, or OTf, OMs or OTs) in the presence of base (base includes but is not limited to Cs₂CO₃, K₂CO₃, K₃PO₄, NaH, LiHMDS). When Q1 is H, the synthesis can commence from protected amino acid XV-3. Next, reaction of XV-3 with the amine XV-4 by activation with a chloroformate in the presence of base (including but not limited to Et₃N, (iPr)₂NEt, and N-methylmorpholine) or under standard coupling conditions described in Chem. Rev. 2011, 111, 11, 6557-6602 by Ayman El-Faham and Fernando Albericio provides the amide XV-5. The PG can be removed under standard deprotection conditions which are summarized in Greene's Protective Groups in Organic Synthesis, 5th Edition, Peter G. M. Wuts, Wiley 2014 to afford amine XV-6 or a related ammonium salt. The intermediate XV-6 is reacted with sulfonyl chloride XV-7 to provide the sulfonamide XV-8 (X₂ is commonly, but not limited to F; X₃ is commonly, but not limited to F and C₁; X₄ is commonly, but not limited to Br) in the presence of base (including but not limited to Et₃N, (iPr)₂NEt, and N-methylmorpholine). Reduction of amide XV-8 to the corresponding secondary amine XV-9 occurs in the presence of a common reducing reagent not limited to borane dimethyl sulfide complex, lithium aluminum hydride, or alane N, N-dimethylethylamine complex. In the presence of base (including but not limited to ((iPr)₂NEt, Cs₂CO₃, K₂CO₃, K₃PO₄) the amine XV-9 undergoes cyclization to XV-10 while losing H—X₂. At this stage, XV-10 can be reacted directly with XV-12 (M1 is H, halogen, —B(OH)₂, —BF3K, —B(OR)2, SnR3, OTf, or OMs) in the presence of a metal containing catalyst and base (base includes but is not limited to K₂CO₃, Cs₂CO₃, K₃PO₄) to form XV-13 using one of the methods described in de Meijere, A., Brase, S., & Oestreich, M. (Eds.). (2014). Metal catalyzed cross-coupling reactions and more: volumes 1-3. Wiley-VCH Verlag. https://doi.org/10.1002/9783527655588. Alternatively, XV-10 can be converted to XV-11 (M2 is —B(OH)2, —BF3K, —B(OR)2, SnR3) then reacted with XV-12 in the presence of a metal containing catalyst and base as described in de Meijere, A., Brase, S., & Oestreich, M. (Eds.). (2014). Metal catalyzed cross-coupling reactions and more: volumes 1-3. Wiley-VCH Verlag. https://doi.org/10.1002/9783527655588. The PG₂ is commonly but not limited to Ac or is H (S is not protected). In some examples PG₂ is Ac and R₁′ is an ester, and removal of Ac under basic conditions simultaneously induces the conversion of R₁′ to a carboxylic acid. Reaction of intermediate XV-13 in the presence of base results in loss of H—X₃ and cyclization to form XVI.

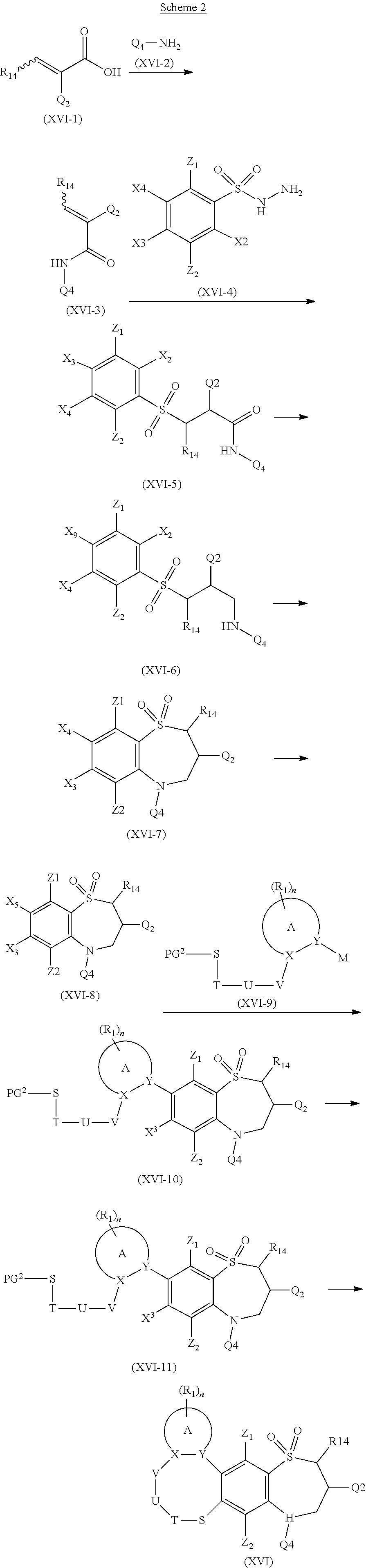

Scheme 2 illustrates a general method to prepare the compound of formula XVI, wherein R₁₄, Q2, Q4, Z1, Z2, S, T, U, V, X, Y, A, and R₁ are as previously defined. Acrylic acid derivative XVI-1 is reacted with amine XVI-2 to form acrylamide derivative XVI-3 by activation with a chloroformate in the presence of base (including but not limited to Et₃N, (iPr)₂NEt, and N-methylmorpholine) or under standard coupling conditions described in Chem. Rev. 2011, 111, 11, 6557-6602 by Ayman El-Faham and Fernando Albericio. Intermediate XVI-3 can be reacted with sulfonyl hydrazide XVI-4 to form sulfone XVI-6 (X₂ is commonly, but not limited to F; X₃ is commonly, but not limited to F and Cl; X₄ is commonly, but not limited to Br) under conditions similar to those reported in Green Chem., 2014, 16, 4106-4109 by Wang et al. Reduction of XVI-5 to amine XVI-6 occurs in the presence of a reducing reagent, including, but not limited to borane dimethyl sulfide complex. In the presence of base (including but not limited to ((iPr)2NEt, Cs2CO3, K2CO3, K3PO4) amine XVI-6 undergoes cyclization with loss of H—X₂ to give intermediate XVI-7. Intermediate XVI-7 can be reacted directly with XVI-9 (M defined as H, halogen, —B(OH)2, —BF3K, —B(OR)2, SnR3, OTf, or OMs) to form XVI-10 in the presence of a transition metal containing catalyst and base using one of the methods described in de Meijere, A., Brase, S., & Oestreich, M. (Eds.). (2014). Metal catalyzed cross-coupling reactions and more: volumes 1-3. Wiley-VCH Verlag. https://doi.org/10.1002/9783527655588. Alternatively, XVI-7 can first be transformed into XVI-8, wherein X5 is defined as —B(OH)2, —BF3K, —B(OR)2, SnR3, and then reacted with XVI-9 to form XVI-10. The PG2 is commonly but not limited to Ac or is H (S is not protected). In some examples PG2 is Ac and R1′ is an ester, and removal of Ac under basic conditions simultaneously induces the conversion of R1′ to a carboxylic acid. Reaction of XVI-11 results in loss of X₃—H and cyclization to form XVI.

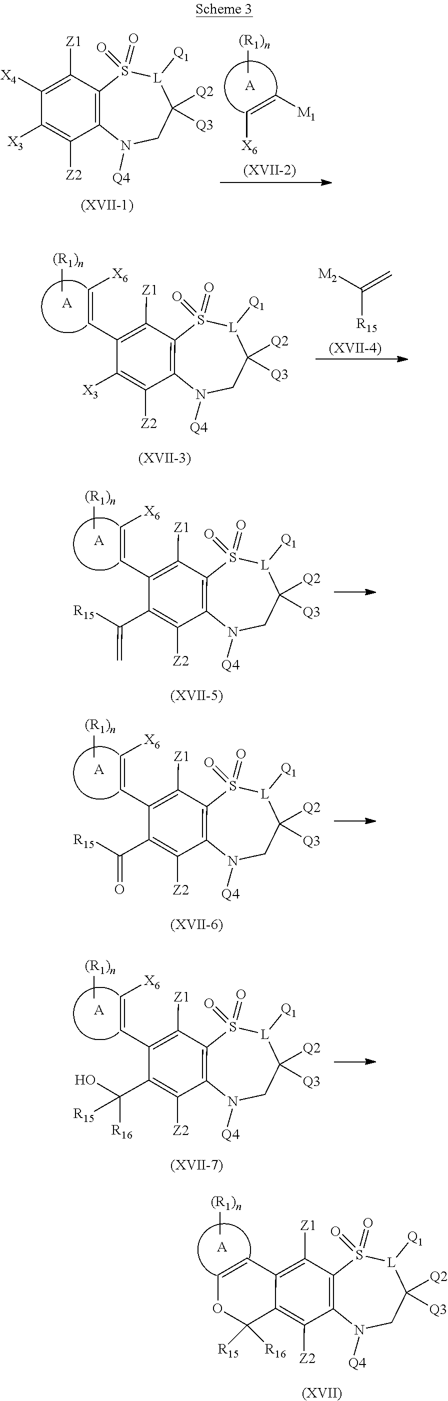

Scheme 3 illustrates a general method to prepare the compound of formula XVII. Reaction of XVII-1, wherein L, Q2, Q3, Q4, Z1, Z2, X3, and X4 are as defined previously, with XVII-2, wherein X-6 is commonly but not limited to F and M₁ is —B(OH)2, —BF3K, —B(OR)2, SnR₃, in the presence of a metal containing catalyst and base using one of the methods described in de Meijere, A., Brase, S., & Oestreich, M. (Eds.). (2014). Metal catalyzed cross-coupling reactions and more: volumes 1-3. Wiley-VCH Verlag. https://doi.org/10.1002/9783527655588 provides the biaryl intermediate XVII-3. Reaction of intermediate XVII-3 with XVII-4 in the presence of a metal containing catalyst and base provides styrene intermediate XVII-5. Oxidative cleavage of the alkene in XVII-5 provides carbonyl XVII-6. Reaction of carbonyl XVII-6 with hydride reagents including but not limited to NaBH₄, or organometallic reagents (organometallic reagents include but are not limited to R₁₆—MgX, R₁₆—Li, R₁₆—ZnX, (R₁₆)₂Zn, R₁₆—SiR₃, R—B(OH)₂) furnishes alcohol XVII-7. Reaction of XVII-7 with base induces cyclization and elimination of H—X₆.

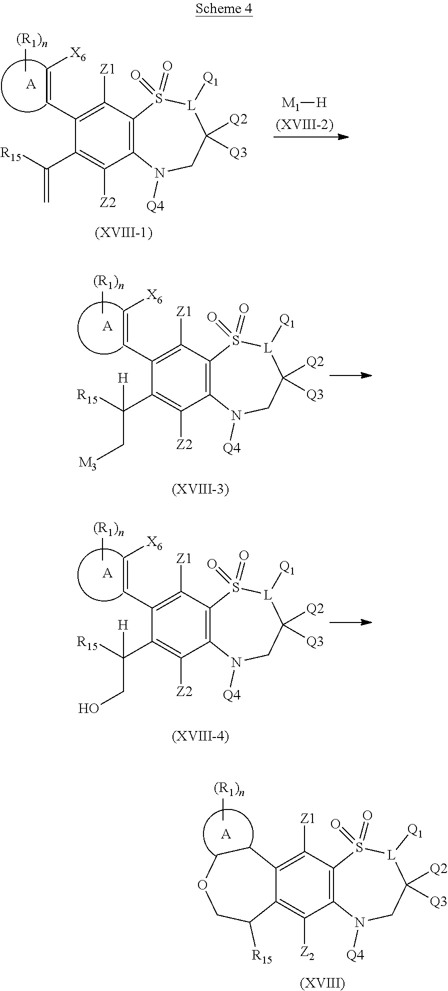

Scheme 4 illustrates a general method to prepare a compound of formula XVIII, wherein L, Q2, Q3, Q4, Z1, Z2, R₁₅, A, and R₁ are as previously defined. Reaction of olefin XVIII-1 with XVIII-2 (M₁ is BR₂, B(OR)₂, BH₂) furnishes intermediate XVIII-3. Oxidation of XVIII-3 produces alcohol XVIII-4. Reaction of XVIII-4 with base induces cyclization and loss of H—X₆ to form XVIII.

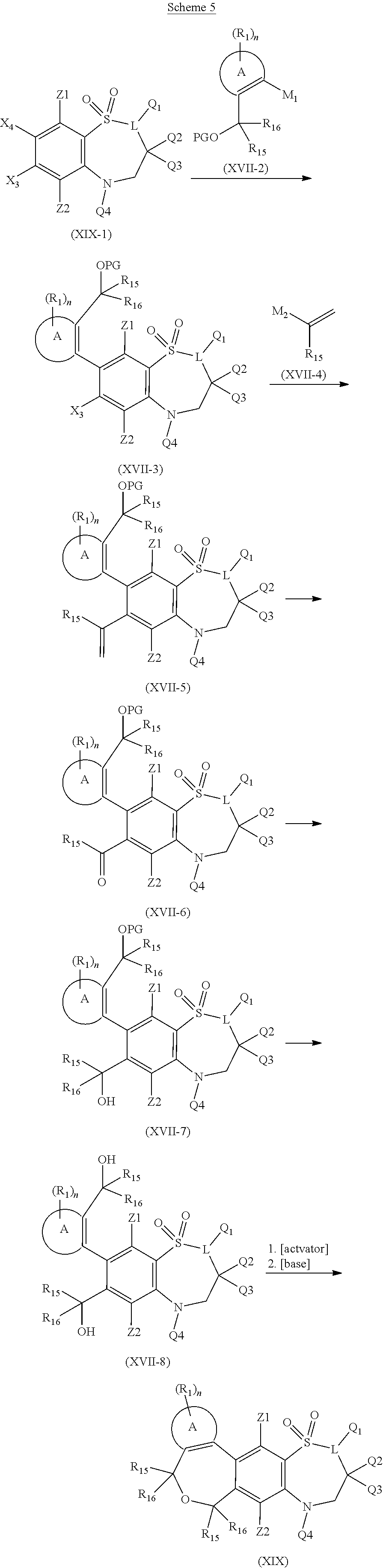

Scheme 5 provides a general method to prepare a compound of formula XIX, wherein L, Q2, Q3, Q4, Z1, Z2, R₁₅, R₁₆, and R₁ are as previously defined. PG is a common alcohol protecting group including but not limited to Ac. Intermediate XIX is reacted with XIX-2 (M₁=B(OH)₂, BF₃K, B(OR)₂, halogen, OTf, OTs, OMs) in the presence of a metal containing catalyst and base to provide XIX-3. Intermediate XI3 is reacted with intermediate XIX-4 (M₂=B(OH)₂, BF₃K, B(OR)₂, halogen, OTf, OTs, OMs) to provide alkene XIX-5. Alkene XIX-5 is oxidized to provide carbonyl XIX-6. Reaction of carbonyl XIX-6 with hydride reagents including but not limited to NaBH₄, or organometallic reagents (organometallic reagents include but are not limited to R₁₆—MgX, R₁₆—Li, R₁₆—ZnX, (R₁₆)₂Zn, R₁₆—SiR₃, R—B(OH)₂) in the presence or absence of a transition metal catalyst and base furnishes alcohol XIX-7. Removal of PG affords diol XIX-7. Reaction of XIX-7 with alcohol activating groups including but not limited to triflic anhydride, mesyl chloride, and tosyl chloride, followed by treatment with base provides XIX.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

### EXAMPLES

The compounds and processes of the present invention will be better understood in connection with the following examples, which are intended as an illustration only and not limiting of the scope of the invention. Starting materials were either available from a commercial vendor or produced by methods well known to those skilled in the art.

While this invention has been particularly shown and described with references to preferred embodiments thereof, it will be understood by those skilled in the art that various changes in form and details may be made therein without departing from the scope of the invention encompassed by the appended claims.

### General Conditions:

Mass spectra were run on LC-MS systems using electrospray ionization. These were Agilent 1290 Infinity II systems with an Agilent 6120 Quadrupole detector. Spectra were obtained using a ZORBAX Eclipse XDB-C₁₈ column (4.6×30 mm, 1.8 micron). Spectra were obtained at 298K using a mobile phase of 0.1% formic acid in water (A) and 0.1% formic acid in acetonitrile (B). Spectra were obtained with the following solvent gradient: 5% (B) from 0-1.5 min, 5-95% (B) from 1.5-4.5 min, and 95% (B) from 4.5-6 min. The solvent flowrate was 1.2 mL/min. Compounds were detected at 210 nm and 254 nm wavelengths. [M+H]+ refers to mono-isotopic molecular weights.

NMR spectra were run on a Bruker 400 MHz or Bruker 500 MHz spectrometer. Spectra were measured at 298K and referenced using the solvent peak. Chemical shifts for ¹H NMR are reported in parts per million (ppm).

Compounds were purified via reverse-phase high-performance liquid chromatography (RPHPLC) using a Gilson GX-281 automated liquid handling system. Compounds were purified on a Phenomenex Kinetex EVO C18 column (250×21.2 mm, 5 micron), unless otherwise specified. Compounds were purified at 298K using a mobile phase of water (A) and acetonitrile (B) using gradient elution between 0% and 100% (B), unless otherwise specified. The solvent flowrate was 20 mL/min and compounds were detected at 254 nm wavelength.

Alternatively, compounds were purified via normal-phase liquid chromatography (NPLC) using a Teledyne ISCO Combiflash purification system. Compounds were purified on a REDISEP silica gel cartridge. Compounds were purified at 298K and detected at 254 nm wavelength.

### Ex. 1: Synthesis of (R)-10-cyclobutyl-11-methyl-8-phenyl-8,9,10,11-tetrahydro-5H-benzo[3,4]chromeno[7,6-f][1,2,5]thiadiazepine-2-carboxylic acid 12,12-dioxide

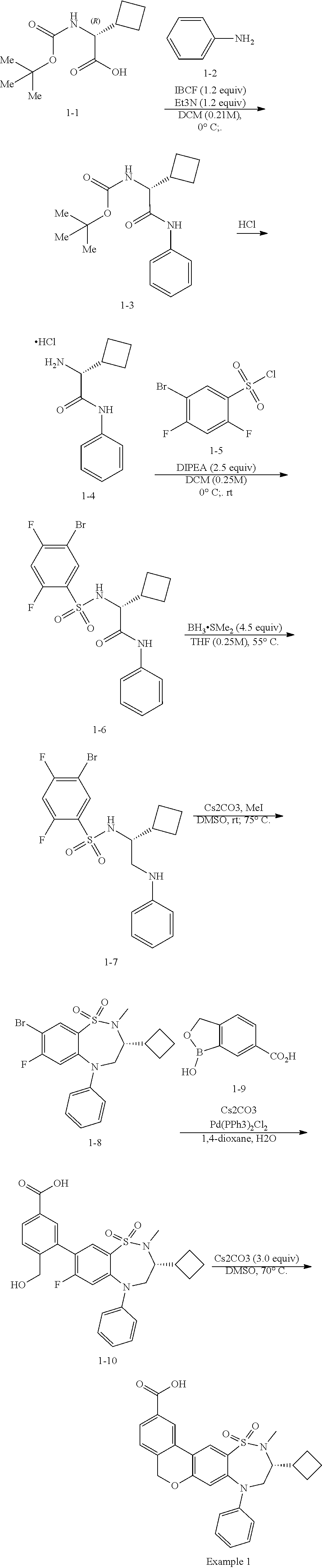

### Step 1-1

In a round-bottomed flask equipped with a stir bar, (2R)-2-(tert-butoxycarbonylamino)-2-cyclobutyl-acetic acid (1.00 g, 8.6 mmol, 1.0 equiv, CAS #155905-78-5) was dissolved in dichloromethane (40 mL, 0.21M). The reaction mixture was cooled to 0° C. using an ice and water bath. To the resulting solution was added isobutyl chloroformate (1.35 mL, 10.3 mmol, 1.2 equiv), followed immediately by triethylamine (1.44 mL), 10.3 mmol, 1.2 equiv). The resulting mixture was stirred for 30 min at 0° C. prior to the addition of aniline (0.940 mL, 10.3 mmol, 1.2 equiv). The resulting mixture was stirred for 19 h while being allowed to warm to room temperature. At this time, analysis of the reaction mixture by LC-MS indicated full conversion to the desired anilide. The solvent was removed in vacuo and the residue was purified by silica gel column chromatography (cyclohexane/ethyl acetate, 0 to 30% ethyl acetate) to afford tert-butyl (R)-(1-cyclobutyl-2-oxo-2-(phenylamino)ethyl)carbamate 1-3 (2.6 g, quantitative yield). ESI MS m/ [symbol] =327.2 [M+Na]+.

### Step 1-2

In a round-bottomed flask equipped with a stir bar tert-butyl (R)-(1-cyclobutyl-2-oxo-2-(phenylamino)ethyl)carbamate (2.6 g, 8.6 mmol, 1.0 equiv) was treated with HCl (4M dioxane, 32.4 mL, 15.0 equiv). After stirring for 1 h at room temperature, analysis of the reaction mixture indicated full conversion to the desired amine-hydrochloride salt. Concentration of the reaction mixture afforded (R)-2-amino-2-cyclobutyl-N-phenylacetamide hydrochloride, which was used in the next step without purification. ESI MS m/ [symbol] =205.0 [M+H]+.

### Step 1-3

In a round-bottomed flask equipped with a stir bar, (R)-2-amino-2-cyclobutyl-N-phenylacetamide hydrochloride (2.08 g, 8.6 mmol, 1.0 equiv) was suspended in dichloromethane (35 mL, 0.25M). The reaction mixture was cooled to 0° C. using an ice and water bath. Next, N,N-diisopropylethylamine (3.76 mL, 21.6 mmol, 2.5 equiv) was added. Once the resulting mixture had become homogenous, 5-bromo-2,4-difluoro-benzenesulfonyl chloride (2.52 g, 8.6 mmol, 1.0 equiv, CAS #287172-61-6) was added in a single portion. The reaction mixture was stirred overnight while being allowed to warm to room temperature. After 16 h, LC-MS analysis indicated full conversion. The solvent was removed in vacuo, and the residue was purified by silica gel column chromatography (cyclohexane, ethyl acetate, 0 to 40% ethyl acetate) to afford (R)-2-((5-bromo-2,4-difluorophenyl)sulfonamido)-2-cyclobutyl-N-phenylacetamide (2.60 g, 5.66 mmol, 66% yield). ESI MS m/ [symbol] -=461.0 [M+H]+.

### Step 1-4

In a round-bottomed flask equipped with a reflux condenser and stir bar, (R)-2-((5-bromo-2,4-difluorophenyl)sulfonamido)-2-cyclobutyl-N-phenylacetamide (2.60 g, 5.66 mmol) was dissolved in tetrahydrofuran (22.6 mL, 0.25M) under a nitrogen atmosphere. The solution was charged with borane-dimethyl sulfide complex (2.42 mL, 25.5 mmol, 4.5 equiv) and then heated at 55° C. for 16 h. Upon cooling to room temperature, the mixture was further cooled to 0° C. using an ice and water bath and was then slowly quenched with water (20 mL). After the effervescence had subsided, the mixture was further diluted with water (175 mL). The aqueous phase was extracted three times with ethyl acetate (60 mL per extraction), and the combined organic layers were dried over sodium sulfate. Concentration afforded crude (R)-5-bromo-N-(1-cyclobutyl-2-(phenylamino)ethyl)-2,4-difluorobenzenesulfonamide which was used in the next step without purification. ESI MS m/ [symbol] =447.0 [M+H]+.

### Step 1-5

In a round-bottomed flask equipped with a stir bar, (R)-5-bromo-N-(1-cyclobutyl-2-(phenylamino)ethyl)-2,4-difluorobenzenesulfonamide (2.60 g, 5.84 mmol, 1.0 equiv) was dissolved in dimethyl sulfoxide (29.2 mL, 0.2M). Cesium carbonate (9.51 g, 29.2 mmol, 5.0 equiv) was added. Next iodomethane (0.183 mL, 2.92 mmol, 0.5 equiv) was added. After stirring for 15 min at room temperature, analysis of the reaction mixture indicated partial conversion to the intermediate methyl sulfonamide. Additional iodomethane (0.10 mL) was added, and the mixture was stirred for 30 min at room temperature. At this time, full conversion to the alkylated sulfonamide had occurred as judged by LC-MS analysis. Next, the reaction mixture was heated at 75° C. for 16 h. Upon cooling to room temperature, the reaction was diluted with tert-butyl methyl ether (400 mL) and washed three times with water (75 mL) and once with brine (75 mL). The organic phase was dried over magnesium sulfate. Upon concentration, the crude residue was purified by silica gel column chromatography (cyclohexane/ethyl acetate, 0 to 10% ethyl acetate) to afford (R)-8-bromo-3-cyclobutyl-7-fluoro-2-methyl-5-phenyl-2,3,4,5-tetrahydrobenzo[f][1,2,5]thiadiazepine 1,1-dioxide (1.38 g, 3.14 mmol, 54% yield). ESI MS m/ [symbol] =441.0 [M+H]⁺.

### Step 1-6

In a 1 dram vial equipped with a stir bar, (R)-8-bromo-3-cyclobutyl-7-fluoro-2-methyl-5-phenyl 2,3,4,5tetrahydrobenzo[f][1,2,5]thiadiazepine 1,1-dioxide (33.0 mg, 0.075 mmol, 1.0 equiv) was combined with 1-hydroxy-3H-2,1-benzoxaborole-6-carboxylic acid 1-9 (20.0 mg, 0.11 mmol, 1.5 equiv, CAS #1221343-14-1), cesium carbonate (73.4 mg, 0.225 mmol, 3.0 equiv), and bis(triphenylphosphine)palladium(II) dichloride (2.6 mg, 5 mol %, CAS #13965-03-2) were combined neat under a nitrogen atmosphere. Next, 1,4-dioxane (0.75 mL) and water (0.11 mL) were added, and the vial was sealed with electrical tape and heated at 80° C. for 1 h. After 1 h, analysis of the reaction mixture by LC-MS indicated incomplete conversion. The reaction was placed under nitrogen and additional 1-hydroxy-3H-2,1-benzoxaborole-6-carboxylic acid 1-9 (20.0 mg, 0.11 mmol, 1.5 equiv, CAS #1221343-14-1), cesium carbonate (73.4 mg, 0.225 mmol, 3.0 equiv), bis(triphenylphosphine)palladium(II) dichloride (5.0 mg) and water (0.11 mL) were added. The vial was sealed with electrical tape and heated at 90° C. for 30 min. The reaction mixture was quenched with formic acid (0.50 mL) and concentrated. The crude residue was purified by RPHPLC to afford (R)-3-(3-cyclobutyl-7-fluoro-2-methyl-1,1-dioxido-5-phenyl-2,3,4,5-tetrahydrobenzo[f][1,2,5]thiadiazepin-8-yl)-4-(hydroxymethyl)benzoic acid 1-10 (33.2 mg, 0.065 mmol, 87% yield). ESI MS m/ [symbol] =510.9 [M+H]⁺.

### Step 1-7

In a 20 mL scintillation vial equipped with a stir bar, (R)-3-(3-cyclobutyl-7-fluoro-2-methyl-1,1-dioxido-5-phenyl-2,3,4,5-tetrahydrobenzo[f] [1,2,5]thiadiazepin-8-yl)-4-(hydroxymethyl)benzoic acid 1-10 (33.2 mg, 0.065 mmol) was dissolved in dimethyl sulfoxide (0.87 mL, 0.075M). Cesium carbonate (63.6 mg, 0.195 mmol, 3.0 equiv) was added, and the mixture was heated at 70° C. for 16 h. Upon cooling to room temperature, the mixture was purified by RPHPLC to afford (R)-10-cyclobutyl-11-methyl-8-phenyl-8,9,10,11-tetrahydro-5H-benzo[3,4]chromeno[7,6-f][1,2,5]thiadiazepine-2-carboxylic acid 12,12-dioxide as a white solid (6.34 mg, 20% yield). ¹H NMR (400 MHz, DMSO-d₆) δ 8.33 (d, J=1.6 Hz, 1H), 8.29 (s, 1H), 7.98 (dd, J=7.8, 1.5 Hz, 1H), 7.46 (d, J=7.9 Hz, 1H), 7.32-7.24 (m, 2H), 6.98-6.75 (m, 4H), 5.40 (d, J=14.2 Hz, 1H), 5.34 (d, J=14.2 Hz, 1H), 3.99 (d, J=16.1 Hz, 1H), 3.76 (t, J=10.4 Hz, 1H), 2.58 (s, 3H), 2.28-2.19 (m, 1H), 2.12-2.05 (m, 1H), 1.95-1.82 (m, 5H). ESI MS m/ [symbol] =491.0 [M+H]⁺.

### Ex. 2: Synthesis of (R)-10-cyclopropyl-11-methyl-8-phenyl-8,9,10,11-tetrahydro-5H-benzo[3,4]chromeno[7,6-f][1,2,5]thiadiazepine-2-carboxylic acid 12,12-dioxide

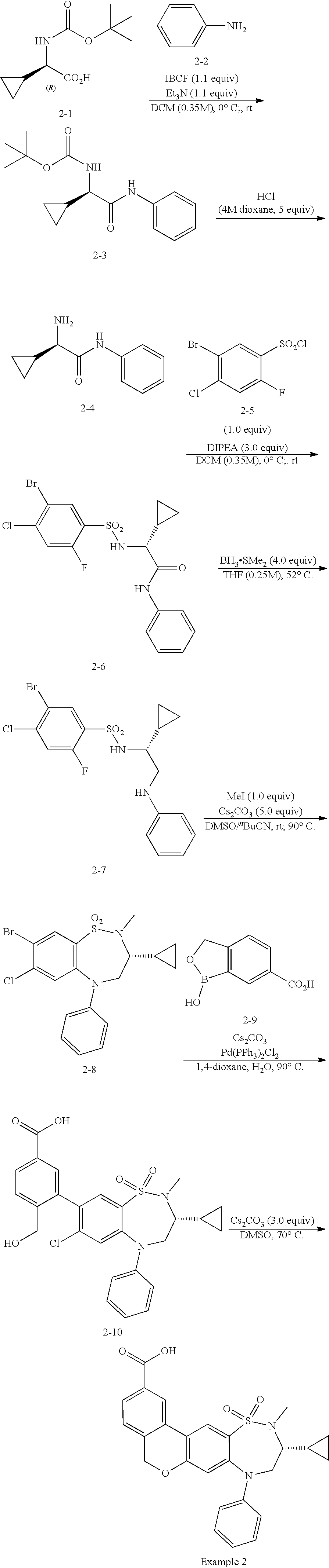

### Step 2-1

In a 40 mL vial equipped with a stir bar, (R)-2-((tert-butoxycarbonyl)amino)-2-cyclopropylacetic acid (1.00 g, CAS #609768-49-2) was dissolved in DCM (13.3 mL, 0.35M). The solution was cooled to 0° C. in an ice and water bath and isobutyl chloroformate (0.67 mL, 1.1 equiv) was added, followed immediately by the addition of triethylamine (0.71 mL, 1.1 equiv). The resulting mixture was stirred for 30 minutes prior to the addition of aniline (0.47 mL, 1.1 equiv). The reaction was stirred for an additional 16 h while being allowed to warm to room temperature. Upon completion, the reaction mixture was concentrated and the crude material was purified by silica gel column chromatography to afford tert-butyl (R)-(1-cyclopropyl-2-oxo-2-(phenylamino)ethyl)carbamate (1.30 g, 97%). ESI MS m/ [symbol] =313.2 [M+Na]⁺.

### Step 2-2

In a 40 mL vial equipped with a stir bar, tert-butyl (R)-(1-cyclopropyl-2-oxo-2-(phenylamino)ethyl)carbamate (1.30 g) was combined with hydrochloric acid (4M in 1,4-dioxane, 5.6 mL. 5 equiv). The mixture was stirred for 3 h and concentrated to afford (R)-2-amino-2-cyclopropyl-N-phenylacetamide hydrochloride (1.02 g, theoretical mass) which was used without purification. ESI MS m/ [symbol] =191.0 [M+H]⁺.

### Step 2-3

In a 40 mL vial equipped with a stir bar, (R)-2-amino-2-cyclopropyl-N-phenylacetamide hydrochloride (1.02 g) was suspended in dichloromethane (12.8 mL, 0.35M). The suspension was cooled in an ice and water bath and N,N-diisopropylethylamine (2.35 mL, 3.0 equiv) was added followed by 5-bromo-4-chloro-2-fluorobenzenesulfonyl chloride (1.38 g, 1.0 equiv, CAS #: 1070972-67-6). The resulting solution was stirred for 3 h and concentrated. The crude residue was purified by silica gel column chromatography to afford (R)-2-((5-bromo-4-chloro-2-fluorophenyl)sulfonamido)-2-cyclopropyl-N-phenylacetamide (1.97 g, 95%). ESI MS m/ [symbol] =461.0 [M+H]⁺.

### Step 2-4

In a 40 mL vial equipped with a stir bar, (R)-2-((5-bromo-4-chloro-2-fluorophenyl)sulfonamido)-2-cyclopropyl-N-phenylacetamide (1.97 g) was dissolved in tetrahydrofuran (13.9 mL, 0.3M) under a nitrogen atmosphere. Borane-dimethyl sulfide complex (1.59 mL, 4.0 equiv) was added, and the mixture was heated at 52° C. for 24 h. Upon cooling to room temperature, the reaction was slowly quenched with water (1.0 mL) and concentrated. The crude residue was purified by silica gel column chromatography to afford (R)-5-bromo-4-chloro-N-(1-cyclopropyl-2-(phenylamino)ethyl)-2-fluorobenzenesulfonamide (1.68 g, 90%). ESI MS m/ [symbol] =447.0 [M+H]⁺.

### Step 2-5

In a 40 mL vial equipped with a stir bar, (R)-5-bromo-4-chloro-N-(1-cyclopropyl-2-(phenylamino)ethyl)-2-fluorobenzenesulfonamide (1.68 g) was dissolved in dimethyl sulfoxide (15.0 mL, 0.25M). Cesium carbonate (6.11 g, 5.0 equiv) was added, followed by a solution of iodomethane (3M in butyronitrile, 1.25 mL, 1.0 equiv). Within 15 minutes, LC-MS indicated full conversion to the N-methylsulfonamide and the mixture was subsequently heated at 90° C. for 14 h. Upon cooling to room temperature, the reaction mixture was concentrated using a Biotage® V-10 evaporator and the crude residue was purified by silica gel column chromatography to afford (R)-8-bromo-7-chloro-3-cyclopropyl-2-methyl-5-phenyl-2,3,4,5-tetrahydrobenzo[f][1,2,5]thiadiazepine 1,1-dioxide (1.09 g, 66% yield). ESI MS m/ [symbol] =440.8 [M+H].

### Step 2-6

In a 1 dram vial equipped with a stir bar, (R)-8-bromo-7-chloro-3-cyclopropyl-2-methyl-5-phenyl-2,3,4,5-tetrahydrobenzo[f][1,2,5]thiadiazepine 1,1-dioxide (60.0 mg, 0.14 mmol, 1.0 equiv), 1-hydroxy-3H-2,1-benzoxaborole-6-carboxylic acid 1-9 (48.0 mg, 0.272 mmol, 2.0 equiv, CAS #1221343-14-1), cesium carbonate (177.0 mg, 0.54 mmol, 4.0 equiv), and bis(triphenylphosphine)palladium(II) dichloride (9.5 mg, 10 mol %, CAS #13965-03-2) were combined neat under a nitrogen atmosphere. Next, 1,4-dioxane (1.1 mL) and water (0.16 mL) were added, and the vial was sealed with electrical tape before being heated at 90° C. for 12 h. Upon cooling to room temperature, the reaction mixture was quenched with formic acid (0.5 mL) and concentrated. Purification of the residue by RPHPLC provided (R)-3-(7-chloro-3-cyclopropyl-2-methyl-1,1-dioxido-5-phenyl-2,3,4,5-tetrahydrobenzo[f][1,2,5]thiadiazepin-8-yl)-4-(hydroxymethyl)benzoic acid (9.9 mg, 14% yield). ESI MS m/ [symbol] =513.0 [M+H]⁺.

### Step 2-7

In a 20 mL vial equipped with a stir bar, (R)-3-(7-chloro-3-cyclopropyl-2-methyl-1,1-dioxido-5-phenyl-2,3,4,5-tetrahydrobenzo[f][1,2,5]thiadiazepin-8-yl)-4-(hydroxymethyl)benzoic acid (9.9 mg, 1.0 equiv) was dissolved in dimethyl sulfoxide (0.84 mL). Cesium carbonate (25.0 mg, 4.0 equiv) was added and the mixture was heated at 90° C. for 19 h. Purification of the reaction mixture by RPHPLC afforded (R)-10-cyclopropyl-11-methyl-8-phenyl-8,9,10,11-tetrahydro-5H-benzo[3,4]chromeno[7,6-f][1,2,5]thiadiazepine-2-carboxylic acid 12,12-dioxide (5.9 mg, 64% yield). ESI MS m/z=477.0 [M+H]⁺. ¹H NMR (400 MHz, DMSO-d₆) δ 8.33 (d, J=1.6 Hz, 1H), 8.29 (s, 1H), 7.98 (dd, J=7.8, 1.6 Hz, 1H), 7.47 (d, J=7.9 Hz, 1H), 7.32-7.19 (m, 2H), 6.91-6.78 (m, 4H), 5.45-5.31 (m, 2H), 4.21 (d, J=16.1 Hz, 1H), 3.68 (s, 1H), 3.02 (t, J=10.0 Hz, 1H), 2.78 (s, 3H), 1.09-1.00 (m, 1H), 0.79-0.64 (m, 2H), 0.49-0.35 (m, 2H).

The following examples were prepared using a procedure similar to that used for Ex. 2:

| Ex # | Structure | MS | NMR |
|---|---|---|---|
| 3 | 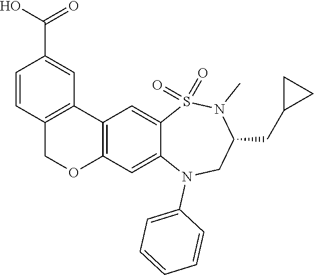 [SMILES: CN1[C@H](CC2CC2)CN(c2ccccc2)c2cc3c(cc2S1(=O)=O)-c1cc(C(=O)O)ccc1CO3] | [M + 1] 491.0 | ¹H NMR (400 MHz, dimethylsulfoxide- d₆) δ 8.32-8.23 (m, 2H), 7.97 (dd, J = 7.8, 1.5 Hz, 1H), 7.46 (d, J = 7.9 Hz, 1H), 7.31-7.27 (m, 2H), 6.91-6.83 (m, 4H), 5.41-5.32 (m, 2H), 4.20 (d, J = 16.1 Hz, 1H), 3.93-3.91 (m, 1H), 3.527- 3.47 (m, 1H), 2.68 (s, 3H), 1.76-1.69 (m, 1H), 1.40-1.33 (m, 1H), 0.92-0.89 (m, 1H), 0.57-0.53 (m, 2H), 0.20-0.17 (m, 2H). |

### Ex. 4: Synthesis of (R)-10-isobutyl-11-methyl-8-phenyl-8,9,10,11-tetrahydro-5H-benzo[3,4]chromeno[7,6-f][1,2,5]thiadiazepine-2-carboxylic acid 12,12-dioxide

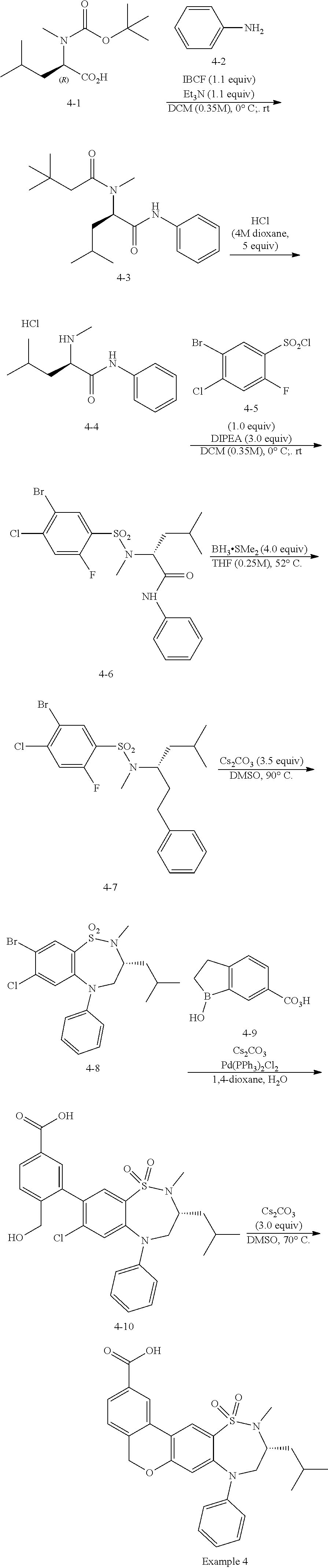

### Step 4-1

In a 40 mL vial equipped with a stir bar, a solution of N-(tert-butoxycarbonyl)-N-methyl-D-leucine (1.10 g, 1.0 equiv, CAS #89536-84-5) was dissolved in dichloromethane (0.3M, 12.8 mL). The solution was cooled to 0° C. using an ice and water bath. Isobutyl chloroformate (0.710 mL, 1.2 equiv) was then added, followed immediately by triethylamine (0.750 mL, 1.2 equiv). After stirring for 30 minutes at 0° C., aniline (0.49 mL, 1.2 equiv) was added. The resulting mixture was stirred for 16 h while being allowed to warm to room temperature. Upon completion, the reaction mixture was concentrated and the residue was purified by silica gel column chromatography (cyclohexanes/ethyl acetate, 0 to 20% ethyl acetate) to provide tert-butyl (R)-methyl(4-methyl-1-oxo-1-(phenylamino)pentan-2-yl)carbamate (1.35 g, 94% yield). ESI MS m/z=343.1 [M+Na]⁺.

### Step 4-2

In a 40 mL vial equipped with a stir bar, tert-butyl (R)-methyl(4-methyl-1-oxo-1-(phenylamino)pentan-2-yl)carbamate (1.35 g) was treated with hydrochloric acid (4M dioxane solution, 5.0 equiv, 5.3 mL). After stirring for 1.5 h, LC-MS analysis of crude reaction mixture indicated full conversion to the desired unprotected amine. Concentration of the reaction mixture afforded methyl-D-leucine hydrochloride (1.09 g) that was used in the next step without purification. ESI MS m/z=146.1 [M+H]⁺.

### Step 4-3

In a 40 mL vial equipped with a stir bar, methyl-D-leucine hydrochloride (1.09 g) was suspended in dichloromethane (12.1 mL, 0.35M). The mixture was cooled to 0° C. in an ice and water bath, and N,N-diisopropylethylamine (2.21 mL, 3.0 equiv) was added, followed by 5-bromo-4-chloro-2-fluorobenzenesulfonyl chloride (1.30 g, 1.0 equiv, CAS #: 1070972-67-6). After stirring for 16 h while being allowed to warm to room temperature, the reaction mixture was concentrated, and the crude residue was purified by silica gel column chromatography (cyclohexane/ethyl acetate) to afford (R)-2-((5-bromo-4-chloro-2-fluoro-N-methylphenyl)sulfonamido)-4-methyl-N-phenylpentanamide (2.08 g). ESI MS m/z=491.1 [M+H]⁺.

### Step 4-4

In a 40 mL vial equipped with a stir bar, (R)-2-((5-bromo-4-chloro-2-fluoro-N-methylphenyl)sulfonamido)-4-methyl-N-phenylpentanamide (2.08 g, 1.0 equiv) was dissolved in tetrahydrofuran (14.1 mL, 0.3M) under a nitrogen atmosphere. Borane-dimethyl sulfide complex (1.61 mL, 4.0 equiv) was added, and the resulting mixture was heated at 52° C. for 12 h. Upon cooling to room temperature, the mixture was further cooled in an ice and water bath and slowly quenched with water (1.0 mL). The mixture was concentrated and the residue was purified by silica gel column chromatography (cyclohexane/ethyl acetate, 0 to 35% ethyl acetate) to afford (R)-5-bromo-4-chloro-2-fluoro-N-methyl-N-(4-methyl-1-(phenylamino)pentan-2-yl)benzenesulfonamide (1.60 g, 79% yield). ESI MS m/z=477.1 [M+H]⁺.

### Step 4-5

In a 40 mL vial equipped with a stir bar, (R)-5-bromo-4-chloro-2-fluoro-N-methyl-N-(4-methyl-1-(phenylamino)pentan-2-yl)benzenesulfonamide (1.60 g, 1.0 equiv) was dissolved in dimethyl sulfoxide (13.4 mL, 0.25M). To this solution was added cesium carbonate (3.81 g, 3.5 equiv). The resulting mixture was heated at 90° C. for 6 h. The reaction mixture was concentrated on a BIOTAGE® V-10 evaporator and purified by silica gel column chromatography (cyclohexane/ethyl acetate, 0 to 15% ethyl acetate) to afford (R)-8-bromo-7-chloro-3-isobutyl-2-methyl-5-phenyl-2,3,4,5-tetrahydrobenzo[f][1,2,5]thiadiazepine 1,1-dioxide (790 mg, 52% yield).

### Step 4-6

In a 1 dram vial equipped with a stir bar, (R)-8-bromo-7-chloro-3-isobutyl-2-methyl-5-phenyl-2,3,4,5-tetrahydrobenzo[f][1,2,5]thiadiazepine 1,1-dioxide (50.0 mg, 1.0 equiv), cesium carbonate (142.3 mg, 4.0 equiv), 1-hydroxy-3H-2,1-benzoxaborole-6-carboxylic acid 1-9 (38.9 mg, 2.0 equiv, CAS #1221343-14-1), and bis(triphenylphosphine)palladium(II) dichloride (7.7 mg, 10 mol %, CAS #13965-03-2) were combined neat under a nitrogen atmosphere. Next, 1,4-dioxane (1.1 mL) and water (0.16 mL) were added, the vial was sealed with electrical tape, and the mixture was heated at 90° C. for 3 h. Upon cooling to room temperature, the mixture was quenched with formic acid (0.5 mL) and concentrated. Purification by RPHPLC afforded (R)-3-(7-chloro-3-isobutyl-2-methyl-1,1-dioxido-5-phenyl-2,3,4,5-tetrahydrobenzo[f][1,2,5]thiadiazepin-8-yl)-4-(hydroxymethyl)benzoic acid (12.0 mg, 21% yield). ESI MS m/z=527.0 [M−H]−.

### Step 4-7

In a 20 mL vial equipped with a stir bar, (R)-3-(7-chloro-3-isobutyl-2-methyl-1,1-dioxido-5-phenyl-2,3,4,5-tetrahydrobenzo[f][1,2,5]thiadiazepin-8-yl)-4-(hydroxymethyl)benzoic acid (12.0 mg) was dissolved in dimethyl sulfoxide (1.0 mL, 0.023M). Cesium carbonate (29.6 mg, 4.0 equiv) was added, and the mixture was heated at 70° C. for 18 h. Purification of the crude reaction mixture by RPHPLC afforded (R)-10-isobutyl-11-methyl-8-phenyl-8,9,10,11-tetrahydro-5H-benzo[3,4]chromeno[7,6-f][1,2,5]thiadiazepine-2-carboxylic acid 12,12-dioxide (3.5 mg, 31% yield). ESI MS m/z=493.0 [M+H]⁺. ¹H NMR (400 MHz, DMSO-d₆) δ 8.36-8.23 (m, 2H), 7.97 (dd, J=7.8, 1.5 Hz, 1H), 7.45 (d, J=7.9 Hz, 1H), 7.30-7.22 (m, 2H), 6.91-6.80 (m, 4H), 5.44-5.29 (m, 2H), 4.13-3.90 (m, 2H), 2.61 (s, 3H), 1.74 (d, J H 7.6 Hz, 1H), 1.64-1.51 (i, 1H), 1.39 (ddd, J=14.3, 9.3, 5.3 Hz, 1H), 0.97 (d, J 6.6 Hz, 3H), 0.94 (d, J 6.5 Hz, 3H).

The following examples were prepared using a procedure similar to that used for Ex. 4:

| Ex # | Structure | MS | NMR |
|---|---|---|---|
| 5 | 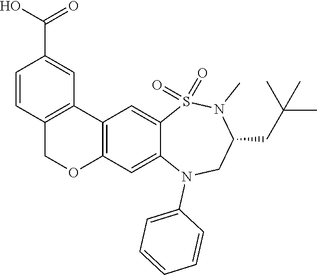 [SMILES: CN1[C@H](CC(C)(C)C)CN(c2ccccc2)c2cc3c(cc2S1(=O)=O)-c1cc(C(=O)O)ccc1CO3] | [M + 1] 507.2 | ¹H NMR (400 MHz, dimethylsulfoxide-d₆) δ 8.34-8.24 (m, 2H), 7.96 (dd, J = 7.8, 1.5 Hz, 1H), 7.44 (d, J = 7.9 Hz, 1H), 7.31-7.27 (m, 2H), 6.92- 6.89 (m, 3H), 6.80 (s, 1H), 5.43-5.27 (m, 2H), 4.14- 3.88 (m, 2H), 3.55 (br s, 1H), 2.66 (s, 3H), 1.76-1.70 (m, 1H), (dd, J = 14.8, 3.2 Hz, 1H), 1.00 (s, 9H). |
| 6 | 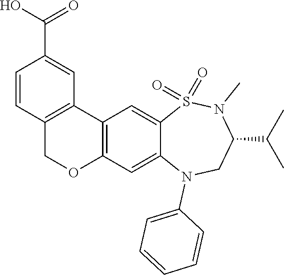 [SMILES: CC(C)[C@@H]1CN(c2ccccc2)c2cc3c(cc2S(=O)(=O)N1C)-c1cc(C(=O)O)ccc1CO3] | [M + 1] 479.2 | ¹H NMR (400 MHz, dimethylsulfoxide-d₆) δ 13.30 (br s, 1H), 8.35-8.21 (m, 2H), 7.96 (dd, J = 7.9, 1.5 Hz, 1H), 7.46 (d, J = 7.9 Hz, 1H), 7.34-7.30 (m, 2H), 6.99- 6.94 (m, 3H), 6.71 (s, 1H), 5.43-5.27 (m, 2H), 4.34 (d, J = 16.2 Hz, 1H), 3.65 (s, 1H), 2.71 (s, 3H), 1.95- 1.86 (m, 1H), 1.08 (d, J = 6.5 Hz, 3H), 1.04 (d, J = 6.5 Hz, 3H) |

### Ex. 7: Synthesis of (R)-10-butyl-11-methyl-8-phenyl-8,9,10,11-tetrahydro-5H-benzo[3,4]chromeno[7,6-f][1,2,5]thiadiazepine-2-carboxylic acid 12,12-dioxide

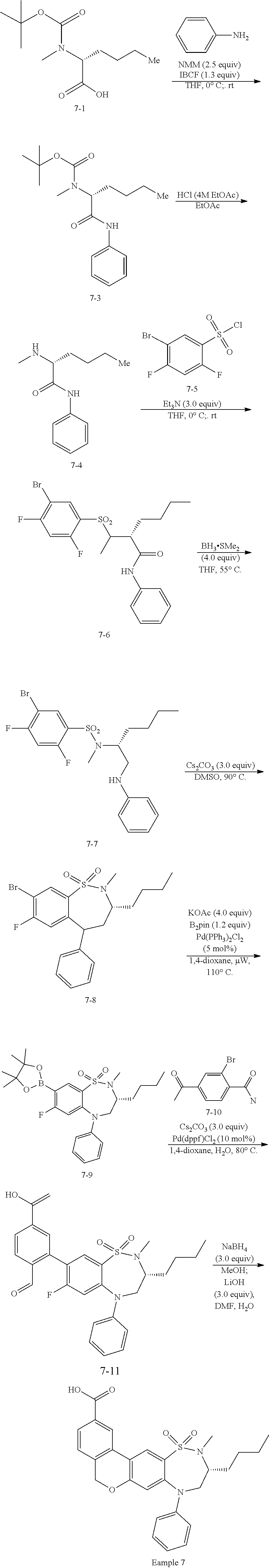

### Step 7-1

To a solution of (R)-2-((tert-butoxycarbonyl)(methyl)amino)hexanoic acid (104 g, 362 mmol, 1 equiv) in THF (1036 mL) was added NMM (91.4 g, 903.8 mmol, 2.5 eq) at 25° C. under a nitrogen atmosphere. The mixture was cooled to 0° C., and isobutyl chloroformate (63.8 g, 467.0 mmol, 1.3 eq) was added dropwise at 0° C. The mixture was then stirred for 15 min. Aniline (35.4 g, 379.6 mmol, 1.05 eq) was then added dropwise at 0° C. The reaction was monitored by HPLC until consumption of the starting material was observed. The mixture was then filtered, and the filtrate was concentrated. The residue was purified by silica gel column chromatography (Heptane:EtOAc 10:1) to afford tert-butyl (R)-methyl(1-oxo-1-(phenylamino)hexan-2-yl)carbamate (115.6 g, 89% purity, 81% assay, 81% yield). ¹H NMR (300 MHz, DMSO-d6) δ 9.87 (d, J=54.0 Hz, 1H), 7.60 (d, J=7.7 Hz, 2H), 7.30 (t, J=7.9 Hz, 2H), 7.05 (t, J=7.4 Hz, 1H), 4.56 (d, J=77.9 Hz, 1H), 2.84 (s, 3H), 1.90-1.58 (m, 2H), 1.40 (d, J=3.7 Hz, 9H), 1.35-1.17 (m, 4H), 0.91-0.85 (m, 3H).

### Step 7-2

To a solution of (R)-methyl(1-oxo-1-(phenylamino)hexan-2-yl)carbamate (115.6 g, 80.7% assay, 291 mmol, 1 eq) in EtOAc (578 mL) was added dropwise a solution of HCl in EtOAc (4 M, 218 mL, 873 mmol, 3.0 eq) at 25° C. under a nitrogen atmosphere. The resulting mixture was stirred at 25° C. for 16 h. The reaction was monitored by HPLC until consumption of the starting material was observed. The mixture was then concentrated, and water (1156.0 mL) and MTBE (1156.0 mL) were added. The mixture was stirred for 10 min and the phases were separated. The aqueous phase was adjusted to pH to 9-10 using 10% aqueous potassium carbonate solution, and then extracted with EtOAc. The combined organic phases were washed with brine, dried over Na₂SO₄, and concentrated to afford (R)-2-(methylamino)-N-phenylhexanamide hydrochloride (59.9 g, 99.9% purity, 96.8% assay, 90.4% yield) as a yellow solid. ¹H NMR (300 MHz, DMSO-d6) δ 9.81 (s, 1H), 7.75-7.56 (m, 2H), 7.30 (t, J=7.9 Hz, 2H), 7.05 (t, J=7.4 Hz, 1H), 3.00 (t, J=6.7 Hz, 1H), 2.24 (s, 3H), 1.61-1.43 (m, 2H), 1.35-1.20 (m, 4H), 0.86 (t, J=6.9 Hz, 3H).

### Step 7-3

To a solution of (R)-2-(methylamino)-N-phenylhexanamide hydrochloride (59.3 g, 96.8% assay, 261 mmol, 1 eq) in THF (593 mL) was added 5-bromo-2,4-difluorobenzenesulfonyl chloride (75.9 g, 261 mmol, 1 eq) and triethylamine (79.1 g, 781.5 mmol, 3.0 eq) at 0° C. under a nitrogen atmosphere. The reaction mixture was then stirred for 16 h at 25° C. The reaction was monitored by HPLC until the starting material was completely consumed. The reaction mixture was filtered through a pad of celite, and the filter cake was washed with EtOAc. The filtrate was concentrated, and the residue was purified by silica gel column chromatography (10:1 heptane/ethyl acetate) to afford (R)-2-((5-bromo-2,4-difluoro-N-methylphenyl)sulfonamido)-N-phenylhexanamide (96.3 g, 98.7% purity, 97.2% assay, 76% yield) as a white solid. ¹H NMR (400 MHz, DMSO-d6) δ 9.97 (s, 1H), 8.08 (t, J=7.4 Hz, 1H), 7.78-7.62 (m, 1H), 7.37 (d, J=7.7 Hz, 2H), 7.25 (t, J=7.9 Hz, 2H), 7.04 (t, J=7.3 Hz, 1H), 4.42 (dd, J=9.0, 6.5 Hz, 1H), 3.09 (d, J=1.7 Hz, 3H), 1.76 (ddd, J=11.7, 8.5, 3.9 Hz, 2H), 1.37-1.10 (m, 4H), 0.86 (t, J=7.1 Hz, 3H).

### Step 7-4

To a solution of (R)-2-((5-bromo-2,4-difluoro-N-methylphenyl)sulfonamido)-N-phenylhexanamide (70.0 g, 97.2% assay, 143.1 mmol, 1.0 eq) in THF (490 mL) was added a solution of BH₃ in DMS (57.2 mL, 10 M, 572.4 mmol, 4.0 eq) at 25° C., and the mixture was heated to 55° C. and stirred for 16 h. The reaction was monitored by HPLC until the starting material was completely consumed. The mixture was then cooled to 0° C., and MeOH (700 mL) was then added in a dropwise fashion. The resulting mixture was stirred for another 30 min at 25° C., then concentrated. The residue was purified by silica gel column chromatography (10:1 heptane/ethyl acetate) to afford (R)-5-bromo-2,4-difluoro-N-methyl-N-(1-(phenylamino)hexan-2-yl)benzenesulfonamide (55.7 g, 99.2% purity, 97.7% assay, 82.4% yield) as a white solid. ¹H NMR (300 MHz, DMSO-d6) δ 7.86 (t, J=7.5 Hz, 1H), 7.61 (dd, J=10.1, 9.0 Hz, 1H), 6.97 (t, J=7.9 Hz, 2H), 6.48 (t, J=7.3 Hz, 1H), 6.30 (d, J=7.7 Hz, 2H), 5.46 (t, J=6.0 Hz, 1H), 3.96-3.74 (m, 1H), 3.12-2.93 (m, 2H), 2.87 (d, J=2.3 Hz, 3H), 1.46 (ddd, J=14.4, 11.5, 5.7 Hz, 2H), 1.30-1.05 (m, 4H).

### Step 7-5

To a solution of (R)-5-bromo-2,4-difluoro-N-methyl-N-(1-(phenylamino)hexan-2-yl)benzenesulfonamide (55.0 g, 97.7% assay, 116.5 mmol, 1.0 eq) in dimethyl sulfoxide (495 mL) was added cesium carbonate (113.9 g, 349.5 mmol, 3.0 eq) at 25° C., and the mixture was heated to 90° C. and stirred for 1.5 h. The reaction was monitored by HPLC until the starting material was completely consumed. The mixture was then cooled to 25° C., and water (495.0 mL) was added. The mixture was extracted with EtOAc. The combined organic extracts were washed with brine, dried with Na₂SO₄, and concentrated. The residue was purified by silica gel column chromatography (15:1 heptane/ethyl acetate) to afford (R)-8-bromo-3-butyl-7-fluoro-2-methyl-5-phenyl-2,3,4,5-tetrahydrobenzo[f][1,2,5]thiadiazepine 1,1-dioxide (27.8 g, 98.7% purity, 97.1% assay, 53% yield) as a white solid. ¹H NMR (300 MHz, CDCl₃) δ 8.15 (d, J=7.6 Hz, 1H), 7.35 (t, J=7.9 Hz, 2H), 7.09 (t, J=7.4 Hz, 1H), 7.02 (d, J=7.8 Hz, 2H), 6.75 (d, J=10.1 Hz, 1H), 4.01 (dd, J=15.2, 2.4 Hz, 1H), 3.88-3.62 (m, 2H), 2.81 (s, 3H), 1.73 (dd, J=15.4, 6.1 Hz, 1H), 1.53-1.30 (m, 5H), 1.00-0.88 (m, 3H).

### Step 7-6

In a 25 mL microwave tube, (R)-8-bromo-3-butyl-7-fluoro-2-methyl-5-phenyl-2,3,4,5-tetrahydrobenzo[f][1,2,5]thiadiazepine 1,1-dioxide (1.00 eq, 800 mg, 1.81 mmol), potassium acetate (4.00 eq, 712 mg, 7.25 mmol), bis(triphenylphosphine)palladium(II) dichloride (64 mg, 0.091 mmol, 5 mol %), and bis(pinacolato)diboron (1.20 eq, 552 mg, 2.18 mmol) were combined neat. The container was flushed with nitrogen and dry 1,4-dioxane was added (18.1 mL). The reaction was heated at 110° C. in a microwave reactor for 40 mins. The resulting mixture was filtered through a celite pad, washing with DCM. The filtrate was concentrated and purified via silica gel column chromatography, eluting with 0-50% ethyl acetate in cyclohexane, to afford (R)-3-butyl-7-fluoro-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (712 mg, 80% yield). ESI MS m/z=489.2 [M+H]⁺.

### Step 7-7

To a vial containing 3-bromo-4-formylbenzoic acid (387 mg, 1.689 mmol, CAS #91760-66-6), (R)-3-butyl-7-fluoro-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (550 mg, 1.126 mmol), cesium carbonate (1101 mg, 3.38 mmol), and PdCl₂(dppf) (82 mg, 0.113 mmol) was added dioxane (9.79 mL) and H₂O (1.469 ml) under a nitrogen atmosphere. The reaction was stirred and heated at 80° C. overnight. The reaction was cooled and then quenched with HCl and extracted with EtOAc. The organic layer was dried, concentrated, then purified by silica gel column chromatography (0-25% ethyl acetate/cyclohexane) to provide (R)-3-(3-butyl-7-fluoro-2-methyl-1,1-dioxido-5-phenyl-2,3,4,5-tetrahydrobenzo[f][1,2,5]thiadiazepin-8-yl)-4-formylbenzoic acid (181 mg, 0.354 mmol, 31.5% yield). [M+H] m/z 511.386.

### Step 7-8

To a solution of (R)-3-(3-butyl-7-fluoro-2-methyl-1,1-dioxido-5-phenyl-2,3,4,5-tetrahydrobenzo[f][1,2,5]thiadiazepin-8-yl)-4-formylbenzoic acid (20 mg, 0.039 mmol) in MeOH (0.196 mL) was added NaBH₄ (4.45 mg, 0.118 mmol). Upon complete conversion to the aldehyde functional group to the corresponding alcohol, the reaction was diluted with DMF (0.196 ml) and LiOH (2.81 mg, 0.118 mmol) was added. The reaction was stirred until complete conversion was observed (heated to 50° C. if necessary). Formic acid (50 μL) was then added, and the mixture was subjected to preparative HPLC to afford (R)-10-butyl-11-methyl-8-phenyl-8,9,10,11-tetrahydro-5H-benzo[3,4]chromeno[7,6-f][1,2,5]thiadiazepine-2-carboxylic acid 12,12-dioxide (12 mg, 0.024 mmol, 62.2% yield). ¹H NMR (400 MHz, DMSO) δ 13.28 (s, 1H), 8.29 (d, J=1.6 Hz, 1H), 8.25 (s, 1H), 7.95 (dd, J=7.7, 1.5 Hz, 1H), 7.44 (d, J=7.9 Hz, 1H), 7.24 (t, J=7.9 Hz, 2H), 6.90-6.82 (m, 3H), 6.81 (s, 1H), 5.41-5.27 (m, 2H), 4.07 (d, J=15.9 Hz, 1H), 3.82-3.76 (m, 1H), 3.47-3.42 (m, 1H), 2.60 (s, 3H), 1.59-1.55 (m, 2H), 1.45-1.27 (m, 4H), 0.91 (t, J=6.9 Hz, 3H). [M+H] m/z 493.334.

The following examples were prepared using a procedure similar to that used for Ex. 7:

| Ex # | Structure | MS | NMR |
|---|---|---|---|
| 8 | 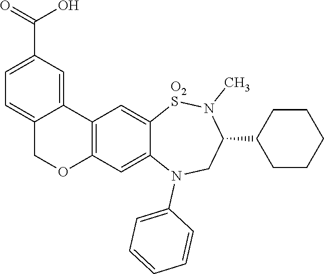 [SMILES: CN1[C@H](C2CCCCC2)CN(c2ccccc2)c2cc3c(cc2S1(=O)=O)-c1cc(C(=O)O)ccc1CO3] | [M + H] m/z 519.359 | ¹H NMR (400 MHz, DMSO) δ 13.27 (s, 1H), 8.27 (s, 1H), 8.21 (s, 1H), 7.92 (d, J = 8.0 Hz, 1H), 7.44-7.38 (m, 1H), 7.27 (t, J = 7.7 Hz, 2H), 6.93-6.88 (m, 3H), 6.71-6.67 (m, 1H), 5.30 (dd, J = 24.5, 14.1 Hz, 2H), 4.36-4.25 (m, 2H), 3.26-3.21 (m, 1H), 2.64 (s, 3H), 2.02-1.86 (m, 1H), 1.74-1.70 (m, 2H), 1.66- 1.51 (m, 2H), 1.25-1.15 (m, 4H), 1.05- 0.93 (m, 2H). |
| 9 | 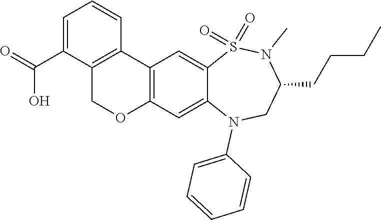 [SMILES: CCCC[C@@H]1CN(c2ccccc2)c2cc3c(cc2S(=O)(=O)N1C)-c1cccc(C(=O)O)c1CO3] | [M + H] m/z 493.200 | ¹H NMR (400 MHz, DMSO) δ 8.26 (s, 1H), 8.10 (d, J = 7.9 Hz, 1H), 7.91 (d, J = 7.8 Hz, 1H), 7.55 (t, J = 7.8 Hz, 1H), 7.24 (t, J = 7.8 Hz, 2H), 6.91-6.80 (m, 3H), 6.78 (s, 1H), 5.60 (dd, J = 39.8, 15.1 Hz, 2H), 4.06 (d, J = 16.1 Hz, 1H), 3.80-3.75 (m, 1H), 3.22 (s, 1H), 2.58 (s, 3H), 1.63-1.50 (m, 2H), 1.42- 1.26 (m, 4H), 0.91 (t, J = 6.8 Hz, 3H). |

### Ex. 10: Synthesis of (R)-10-butyl-3-fluoro-11-methyl-8-phenyl-8,9,10,11-tetrahydro-5H-benzo[3,4]chromeno[7,6-f][11,2,5]thiadiazepine-2-carboxylic acid 12,12-dioxide

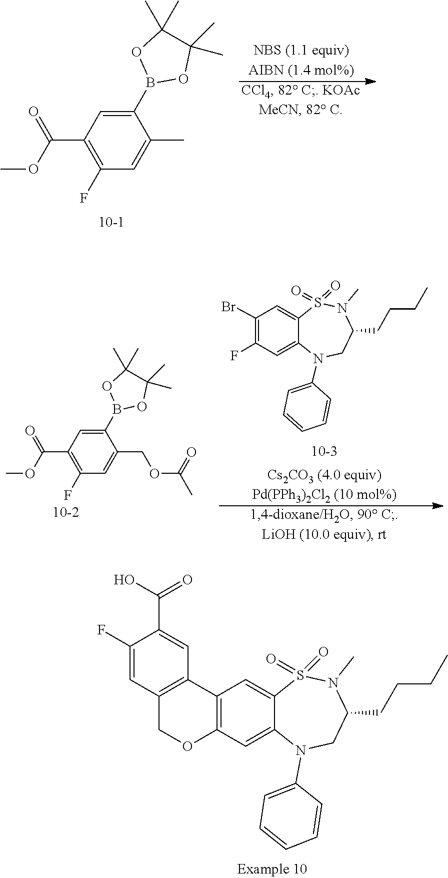

### Step 10-1

In a 50 mL round-bottom flask equipped with a stir bar and reflux condenser, methyl 2-fluoro-4-methyl-S-(4,4,5,5-tetramethyl-1,3,2-dioxaborolan-2-yl)benzoate (736.5 mg, 1.0 equiv, CAS #

1629913-80-9) was dissolved in carbon tetrachloride (10.0 mL, 0.25M) under a nitrogen atmosphere. Next, N-bromosuccinimide (490.2 mg, 1.1 equiv) was added, followed by AIBN (5.8 mg, 1.4 mol %, CAS #78-67-1). The resulting mixture was heated at 82° C. for 6 h. Upon cooling to room temperature, potassium acetate (2.46 g, 10.0 equiv) was added, followed by acetonitrile (10.0 mL). The mixture was heated at 82° C. for 27 h. Upon cooling to room temperature, the reaction mixture was filtered through a pad of celite using ethyl acetate to rinse. Concentration and purification of the residue by RPHPLC afforded methyl 4-(acetoxymethyl)-2-fluoro-5-(4,4,5,5-tetramethyl-1,3,2-dioxaborolan-2-yl)benzoate (395.3 mg, 49% yield). ESI MS m/z=353.0 [M+H]⁺. ¹H NMR (400 MHz, CDCl₃) δ 8.40 (d, J=8.2 Hz, 1H), 7.17 (d, J=12.0 Hz, 1H), 5.41 (s, 2H), 3.93 (s, 3H), 2.15 (s, 3H), 1.34 (s, 12H).

### Step 10-2

In a 1 dram vial equipped with a stir bar, (R)-8-bromo-3-butyl-7-fluoro-2-methyl-5-phenyl-2,3,4,5-tetrahydrobenzo[f][1,2,5]thiadiazepine 1,1-dioxide (30.0 mg, 1.0 equiv), methyl 4-(acetoxymethyl)-2-fluoro-5-(4,4,5,5-tetramethyl-1,3,2-dioxaborolan-2-yl)benzoate (47.9 mg, 2.0 equiv), cesium carbonate (88.6 mg, 4.0 equiv), and bis(triphenylphosphine)palladium(II) chloride (4.8 mg, 10 mol %) were combined neat under a nitrogen atmosphere. Next, 1,4-dioxane (0.68 mL) and water (0.1 mL) were added, and the vial was sealed with electrical tape. The reaction mixture was heated at 90° C. for 4.5 hours. Upon cooling to room temperature, lithium hydroxide (16.3 mg, 10.0 equiv) was added. After stirring for 16 h, the reaction mixture was quenched with formic acid (0.5 mL) and concentrated. Purification of the residue by RPHPLC afforded (R)-10-butyl-3-fluoro-11-methyl-8-phenyl-8,9,10,11-tetrahydro-5H-benzo[3,4]chromeno[7,6-f][1,2,5]thiadiazepine-2-carboxylic acid 12,12-dioxide (3.3 mg, 9% yield). ESI MS m/z=511.2 [M+H]⁺. ¹H NMR (500 MHz, DMSO) δ 13.60 (br s, 1H), 8.27-8.25 (m, 2H), 7.37 (d, J=10.5 Hz, 1H), 7.29-7.26 (m, 2H), 6.90-6.84 (m, 4H), 5.38-5.29 (m, 2H), 4.10 (d, J=16.1 Hz, 1H), 3.85-3.79 (m, 1H), 3.46 (br s, 1H), 2.62 (s, 3H), 1.64-1.57 (m, 2H), 1.41-1.31 (m, 4H), 0.96-0.93 (m, 3H).

### Ex. 11: Synthesis of (R)-3-butyl-7-fluoro-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide

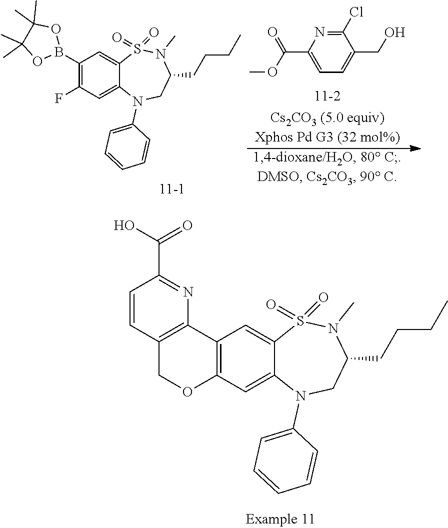

### Step 11-1

A mixture of (R)-3-butyl-7-fluoro-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (20 mg, 0.041 mmol), methyl 6-chloro-5-(hydroxymethyl)picolinate (28.9 mg, 0.143 mmol, CAS #1205671-72-2), XPhos Pd G3 (11 mg, 0.013 mmol, 32 mol %), aqueous cesium carbonate (2 M, 102 μl, 0.205 mmol), and 1,4-dioxane (410 μL) was heated to 80° C. for 98 minutes. The mixture was then cooled to ambient temperature and concentrated in vacuo. The residue was suspended in dimethyl sulfoxide (1 mL) and additional neat cesium carbonate (133 mg, 0.409 mmol) was added. The resulting mixture was heated to 90° C. for 16 h. After this time, the mixture was cooled to ambient temperature and purified by reverse-phase preparative HPLC to afford (R)-10-butyl-11-methyl-8-phenyl-8,9,10,11-tetrahydro-5H-pyrido[3′,2′:3,4]chromeno[7,6-f][1,2,5]thiadiazepine-2-carboxylic acid 12,12-dioxide (1.07 mg, 0.0022 mmol, 5% yield). ¹H NMR (400 MHz, CDCl₃) δ 8.72 (s, 1H), 8.12 (d, J=7.7 Hz, 1H), 7.65 (dt, J=7.9, 0.9 Hz, 1H), 7.40-7.32 (m, 2H), 7.16-7.02 (m, 3H), 6.47 (s, 1H), 5.35-5.20 (m, 2H), 4.11-3.93 (m, 2H), 3.67 (s, 1H), 2.88 (s, 3H), 1.85-1.26 (m, 6H), 0.92 (t, J=7.1 Hz, 3H). [M+H] m/z 489.168.

### Ex. 12: Synthesis of (R)-3,4-difluoro-10-isobutyl-11-methyl-8-phenyl-8,9,10,11-tetrahydro-5H-benzo[3,4]chromeno[7,6-f][1,2,5]thiadiazepine-2-carboxylic acid 12,12-dioxide

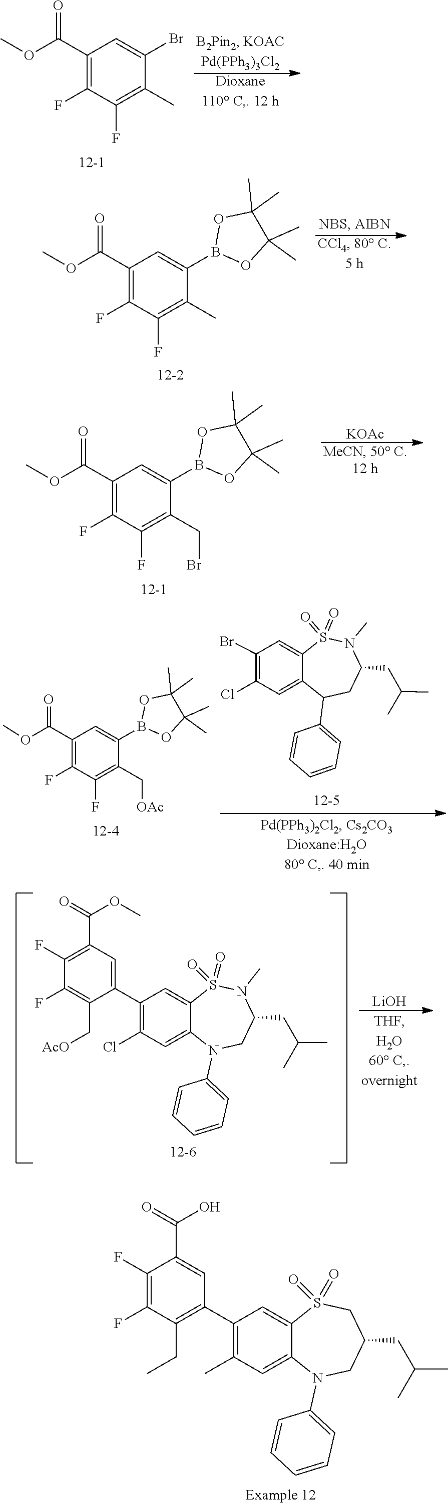

### Step 12-1

In a 250 mL round bottom flask, methyl 5-bromo-2,3-difluoro-4-methylbenzoate (1.0 g, CAS #: 2734772-84-8), potassium acetate (1.5 g, 4.0 equiv), bis(triphenylphosphine)palladium(II) dichloride (132 mg, 0.05 equiv, CAS #: 13965-03-2), and bis(pinacolato)diboron (1.15 g, 1.2 equiv, CAS #: 73183-34-3) were added. The reaction mixture was flushed with nitrogen, and 1,4-dioxane (37.7 mL) was added. Then, the reaction mixture was heated at 110° C. for 12 h. Upon cooling to room temperature, the reaction mixture was filtered through celite and concentrated. Purification of the residue by silica gel column chromatography afforded methyl 2,3-difluoro-4-methyl-5-(4,4,5,5-tetramethyl-1,3,2-dioxaborolan-2-yl)benzoate (1.02 g, 87% yield). [M+H]⁺ 313.1.

### Step 12-2

In a 100 mL flask, methyl 2,3-difluoro-4-methyl-5-(4,4,5,5-tetramethyl-1,3,2-dioxaborolan-2-yl)benzoate (1.02 g) was dissolved in carbon tetrachloride (10.9 mL), followed by addition of N-bromosuccinimide (640 mg, 1.10 equiv, CAS #128-08-5) and azobisisobutyronitrile (13.4 mg, 0.025 equiv, CAS #78-67-1). The reaction mixture was then refluxed at 80° C. for 5 h. Reaction progress was monitored through LC-MS. Upon cooling to room temperature, the reaction mixture was concentrated to provide crude methyl 4-(bromomethyl)-2,3-difluoro-5-(4,4,5,5-tetramethyl-1,3,2-dioxaborolan-2-yl)benzoate that was used in the next step without purification. ESI MS m/z=391.0 [M+H]⁺.

### Step 12-3

The crude 4-(bromomethyl)-2,3-difluoro-5-(4,4,5,5-tetramethyl-1,3,2-dioxaborolan-2-yl)benzoate from Step 2 was dissolved in acetonitrile (10.9 mL). Potassium acetate (3.2 g, 10.0 equiv) was then added, and the reaction mixture was heated at 50° C. for 12 h. After this time, the reaction mixture was filtered through silica gel using ethyl acetate to rinse. Upon concentration, the crude residue was purified by silica gel column chromatography to afford methyl 4-(acetoxymethyl)-2,3-difluoro-5-(4,4,5,5-tetramethyl-1,3,2-dioxaborolan-2-yl)benzoate (740 mg, 61% yield). ESI MS m/z=371.2 [M+H]⁺.

### Step 12-4

In a 2 dram vial equipped with a stir bar, (R)-8-bromo-7-chloro-3-isobutyl-2-methyl-5-phenyl-2,3,4,5-tetrahydrobenzo[f][1,2,5]thiadiazepine 1,1-dioxide (60.0 mg), methyl 4-(acetoxymethyl)-2,3-difluoro-5-(4,4,5,5-tetramethyl-1,3,2-dioxaborolan-2-yl)benzoate (72.8 mg, 1.5 equiv), bis(triphenylphosphine)palladium(II) chloride (9.2 mg, 10 mol %) and cesium carbonate (128 mg, 3.0 equiv) were combined neat under a nitrogen atmosphere. Next, 1,4-dioxane (1.14 ml) and water (0.17 ml) were added, and the reaction mixture was heated at 90° C. for 100 minutes. Upon cooling to room temperature, lithium hydroxide (1M solution in water, 1.3 mL, 10.0 equiv) and tetrahydrofuran (1.3 mL) were added and reaction mixture was stirred overnight at 60° C. Reaction progress was monitored by LC-MS. After 12 h, the reaction was incomplete, and the mixture was then heated at 80° C. for 4 h. Upon cooling to room temperature, the reaction mixture was quenched with 1N HCl, and the aqueous phase was extracted three times with ethyl acetate. The combined organic layers were washed with water and brine, dried over sodium sulfate and concentrated. The crude residue was re-dissolved in 2.0 mL of dimethyl sulfoxide, passed through a 0.45 μm syringe filter, and purified by RPHPLC to afford (R)-3,4-difluoro-10-isobutyl-11-methyl-8-phenyl-8,9,10,11-tetrahydro-5H-benzo[3,4]chromeno[7,6-f][1,2,5]thiadiazepine-2-carboxylic acid 12,12-dioxide (7.9 mg, 11% yield) [M+H]⁺, 529.2. ¹H NMR (400 MHz, dimethylsulfoxide-d₆) δ 13.86 (br, 1H), 8.24 (s, 1H), 8.06 (d, J=5.5 Hz, 1H), 7.36-7.17 (m, 2H), 6.92-6.76 (m, 4H), 5.50-5.31 (m, 2H), 4.04 (d, J=16.0 Hz, 1H), 3.97-3.80 (m, 1H), 3.52-3.35 (br, 1H), 2.58 (s, 3H), 1.77-1.62 (m, 1H), 1.62-1.44 (m, 1H), 1.41-1.28 (m, 1H), 0.93 (d, J=6.7 Hz, 3H), 0.90 (d, J=6.5 Hz, 3H).

The following examples were prepared using a procedure similar to that used for Ex. 12:

| Ex. | | |
|---|---|---|
| # | Structure | MS |
| 13 | 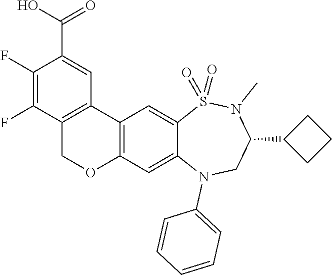 [SMILES: CN1[C@H](C2CCC2)CN(c2ccccc2)c2cc3c(cc2S1(=O)=O)-c1cc(C(=O)O)c(F)c(F)c1CO3] | [M + 1] 527.2 |
| 14 | 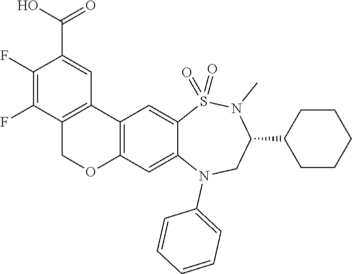 [SMILES: CN1[C@H](C2CCCCC2)CN(c2ccccc2)c2cc3c(cc2S1(=O)=O)-c1cc(C(=O)O)c(F)c(F)c1CO3] | [M + 1] 555.3 |

### Ex. 15: Synthesis of (R)-9-butyl-10-methyl-7-phenyl-7,8,9,10-tetrahydro-4H-thieno[2′,3′:3,4]chromeno[7,6-f][1,2,5]thiadiazepine-2-carboxylic acid 11,11-dioxide

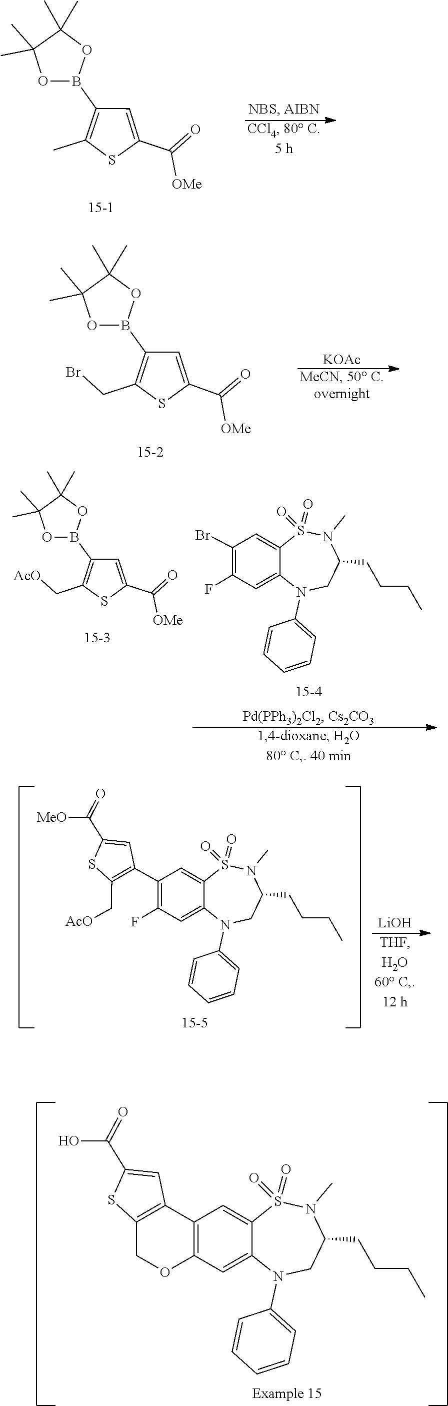

### Step 15-1

In a 100 mL flask, methyl 5-methyl-4-(4,4,5,5-tetramethyl-1,3,2-dioxaborolan-2-yl)thiophene-2-carboxylate (1.0 g, CAS #: 1109284-49-2) was dissolved in dry carbon tetrachloride (11.8 mL), followed by the addition of N-bromosuccinimide (693.8 mg, 1.1 equiv, CAS #128-08-5) and azobisisobutyronitrile (14.5 mg, 0.025 equiv, CAS #78-67-1). The reaction mixture was then refluxed at 80° C. for 5 h. Reaction progress was monitored by LC-MS. Upon cooling to room temperature, the reaction mixture was concentrated to provide methyl 5-(bromomethyl)-4-(4,4,5,5-tetramethyl-1,3,2-dioxaborolan-2-yl)thiophene-2-carboxylate which was used in the next step without purification. ESI MS m/z=361.2 [M+H]⁺.

### Step 15-2

The crude 5-(bromomethyl)-4-(4,4,5,5-tetramethyl-1,3,2-dioxaborolan-2-yl)thiophene-2-carboxylate from Step 15-1 was dissolved in dry acetonitrile (11.8 mL) and potassium acetate (3.5 g, 10.0 equiv) was added to the resulting solution. The reaction mixture was heated at 50° C. overnight. The reaction mixture was then filtered through silica and washed with ethyl acetate. Upon concentration, the crude residue was purified by silica gel column chromatography to afford methyl 5-(acetoxymethyl)-4-(4,4,5,5-tetramethyl-1,3,2-dioxaborolan-2-yl)thiophene-2-carboxylate (1.01 g, 84% yield). ESI MS m/z=341.0 [M+H]⁺.

### Step 15-3

In a 2 dram vial equipped with a stir bar, (R)-8-bromo-3-butyl-7-fluoro-2-methyl-5-phenyl-2,3,4,5-tetrahydrobenzo[f][1,2,5]thiadiazepine 1,1-dioxide (50.0 mg), methyl 5-(acetoxymethyl)-4-(4,4,5,5-tetramethyl-1,3,2-dioxaborolan-2-yl)thiophene-2-carboxylate (46.2 mg, 1.2 equiv), bis(triphenylphosphine)palladium(II) dichloride (4.0 mg, 0.05 equiv, CAS #13965-03-2) and cesium carbonate (110.7 mg, 3.0 equiv) were combined neat under nitrogen atmosphere. Next, 1,4-dioxane (0.97 mL) and water (0.16 mL) were added, and the mixture was stirred at 80° C. for 1 h. Upon cooling to room temperature, lithium hydroxide (1M solution in water, 1.1 mL, 10.0 equiv) and tetrahydrofuran (1.15 mL) were added and the reaction mixture was stirred for 12 h at 60° C. Reaction progress was monitored by LC-MS. After complete conversion, the reaction mixture was quenched with 1N HCl and extracted three times with ethyl acetate. The combined organic layers were washed with water and brine and dried over sodium sulfate and concentrated. The crude residue was re-dissolved in 2.0 mL of dimethyl sulfoxide, passed through a 0.45 μm syringe filter, and purified by RPHPLC to afford (R)-9-butyl-10-methyl-7-phenyl-7,8,9,10-tetrahydro-4H-thieno[2′,3′:3,4]chromeno[7,6-f][1,2,5]thiadiazepine-2-carboxylic acid 11,11-dioxide (11.6 mg, 20% yield). ESI MS m/z=499.2 [M+H]⁺. ¹H NMR (400 MHz, dimethylsulfoxide-d₆) δ 13.34 (br s, 1H), 8.18 (s, 1H), 8.08 (s, 1H), 7.21 (dd, J=8.6, 7.1 Hz, 2H), 6.92-6.73 (m, 4H), 5.61-5.42 (m, 2H), 4.05 (d, J=16.0 Hz, 1H), 3.86-3.68 (m, 1H), 3.47-3.35 (m, 1H), 2.54 (s, 3H), 1.65-1.20 (m, 6H), 0.99-0.84 (m, 3H).

The following examples were prepared using a procedure similar to that used for Ex. 15:

| Ex # | Structure | MS | NMR |
|---|---|---|---|
| 16 | 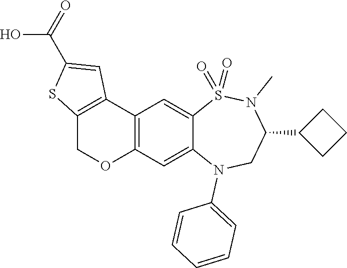 [SMILES: CN1[C@H](C2CCC2)CN(c2ccccc2)c2cc3c(cc2S1(=O)=O)-c1cc(C(=O)O)sc1CO3] | [M + 1] 497.2 | ¹H NMR (400 MHz, dimethylsulfoxide-d₆) δ 13.34 (br, 1H), 8.18 (s, 1H), 8.09 (s, 1H), 7.26-7.18 (m, 2H), 6.84-6.76 (m, 2H), 6.73 (d, J = 8.2 Hz, 2H), 5.52 (q, J = 14.8 Hz, 2H), 3.94 (d, J = 16.1 Hz, 1H), 3.71 (t, J = 10.3 Hz, 1H), 3.35-3.14 (m, 1H), 2.50 (s, 3H), 2.25-2.15 (m, 1H), 2.08-1.76 (m, 6H). |
| 17 | 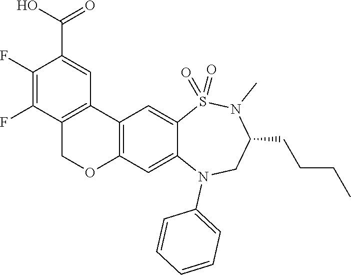 [SMILES: CCCC[C@@H]1CN(c2ccccc2)c2cc3c(cc2S(=O)(=O)N1C)-c1cc(C(=O)O)c(F)c(F)c1CO3] | [M + 1] 529.3 | ¹H NMR (500 MHz, dimethylsulfoxide-d₆) δ 13.86 (br, 1H), 8.23 (s, 1H), 8.06 (d, J = 5.3 Hz, 1H), 7.25 (t, J = 7.9 Hz, 2H), 6.92-6.83 (m, 3H), 6.79 (s, 1H), 5.52-5.33 (m, 2H), 4.07 (d, J = 16.0 Hz, 1H), 3.76 (s, 1H), 3.47 (s, 1H), 2.60 (s, 3H), 1.64-1.48 (m, 2H), 1.46-1.24 (m, 4H), 0.90 (t, J = 6.9 Hz, 3H). |

### Ex. 18: Synthesis of (R)-9-isobutyl-10-methyl-7-phenyl-7,8,9,10-tetrahydro-4H-thieno[2′,3′:3,4]chromeno[7,6-f][1,2,5]thiadiazepine-2-carboxylic acid 11,11-dioxide

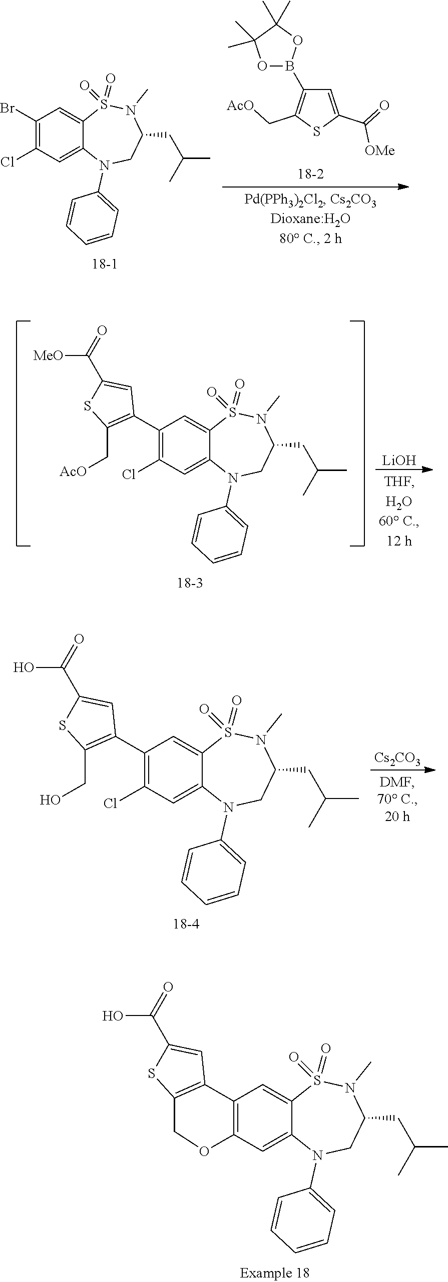

### Step 18-1

In a 2-dram vial equipped with a stir bar, (R)-8-bromo-7-chloro-3-isobutyl-2-methyl-5-phenyl-2,3,4,5-tetrahydrobenzo[f][1,2,5]thiadiazepine 1,1-dioxide (60.0 mg), methyl 5-(acetoxymethyl)-4-(4,4,5,5-tetramethyl-1,3,2-dioxaborolan-2-yl)thiophene-2-carboxylate (53.5 mg, 1.2 equiv), bis(triphenylphosphine)palladium(II) dichloride (4.6 mg, 0.05 equiv, CAS #13965-03-2) and cesium carbonate (128 mg, 3.0 equiv) were combined neat under a nitrogen atmosphere, followed by the addition of 1,4-dioxane (1.14 mL) and water (0.17 mL). The reaction mixture was then stirred at 80° C. for 2 h. Upon cooling to room temperature, lithium hydroxide (1M solution in water, 1.1 mL, 10.0 equiv) and tetrahydrofuran (1.2 mL) were added, and reaction mixture was stirred for 12 h at 60° C. Reaction progress was monitored by LC-MS. After complete conversion, the reaction mixture was quenched with 1N HCl, and extracted three times with ethyl acetate. The combined organic layers were washed with water and brine and dried over sodium sulfate before being concentrated. The crude residue was re-dissolved in 2.0 mL of dimethyl sulfoxide, and purified by reverse phase C₁₈ gold column to afford (R)-4-(7-chloro-3-isobutyl-2-methyl-1,1-dioxido-5-phenyl-2,3,4,5-tetrahydrobenzo[f] [1,2,5]thiadiazepin-8-yl)-5-(hydroxymethyl)thiophene-2-carboxylic acid (36 mg, 51% yield). ESI MS m/z=536.2 [M+H]⁺.

### Step 18-2

(R)-4-(7-chloro-3-isobutyl-2-methyl-1,1-dioxido-5-phenyl-2,3,4,5-tetrahydrobenzo[f][1,2,5]thiadiazepin-8-yl)-5-(hydroxymethyl)thiophene-2-carboxylic acid (36.0 mg) was dissolved in N,N-dimethylformamide (1.3 mL) and cesium carbonate (87.6 mg, 4.0 equiv) was added at room temperature. The resulting mixture was stirred at 70° C. for 20 h. After completion, the reaction was quenched with formic acid (1.0 mL) and concentrated. The crude residue was re-dissolved in 2.0 mL of dimethyl sulfoxide, passed through a 0.45 μm syringe filter, and purified by RPHPLC to afford (R)-9-isobutyl-10-methyl-7-phenyl-7,8,9,10-tetrahydro-4H-thieno[2′,3′:3,4]chromeno[7,6-f][1,2,5]thiadiazepine-2-carboxylic acid 11,11-dioxide (18.7 mg, 56% yield) ESI MS m/z=499.2 [M+H]⁺. ¹H NMR (500 MHz, dimethylsulfoxide-d₆) δ 13.34 (br, 1H), 8.19 (s, 1H), 8.09 (s, 1H), 7.20 (dd, J=8.5, 7.2 Hz, 2H), 6.83-6.74 (m, 4H), 5.59-5.46 (m, 2H), 4.02 (d, J=16.1 Hz, 1H), 3.96-3.86 (m, 1H), 3.34-3.28 (m, 1H), 2.52 (s, 3H), 1.77-1.63 (m, 1H), 1.56-1.47 (m, 1H), 1.40-1.28 (m, 1H), 0.93 (d, J=6.6 Hz, 3H), 0.89 (d, J=6.5 Hz, 3H).

The following examples were prepared using a procedure similar to that used for Ex. 18:

| Ex. | | | |
|---|---|---|---|
| # | Structure | MS | NMR |
| 19 | 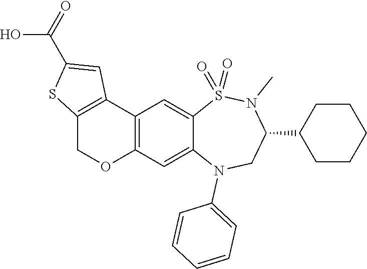 [SMILES: CN1[C@H](C2CCCCC2)CN(c2ccccc2)c2cc3c(cc2S1(=O)=O)-c1cc(C(=O)O)sc1CO3] | [M + 1] 525.4 | ¹H NMR (500 MHz, dimethylsulfoxide-d₆) δ 13.32 (br, 1H), 8.20 (s, 1H), 8.07 (s, 1H), 7.25 (t, J = 7.7 Hz, 2H), 6.95-6.78 (m, 3H), 6.69 (s, 1H), 5.61-5.42 (m, 2H), 4.31 (d, J = 16.1 Hz, 1H), 3.48 (s, 1H), 2.60 (s, 3H), 2.06-1.83 (m, 2H), 1.79-1.47 (m, 4H), 1.32-0.85 (m, 5H) |

### Ex. 20 Synthesis of (R)-9-butyl-10-methyl-7-phenyl-7,8,9,10-tetrahydro-4H-pyrazolo[1″,5″:4′,5′][1,4]oxazino[3′,2′:4,5]benzo[1,2-f][1,2,5]thiadiazepine-2-carboxylic acid 11,11-dioxide

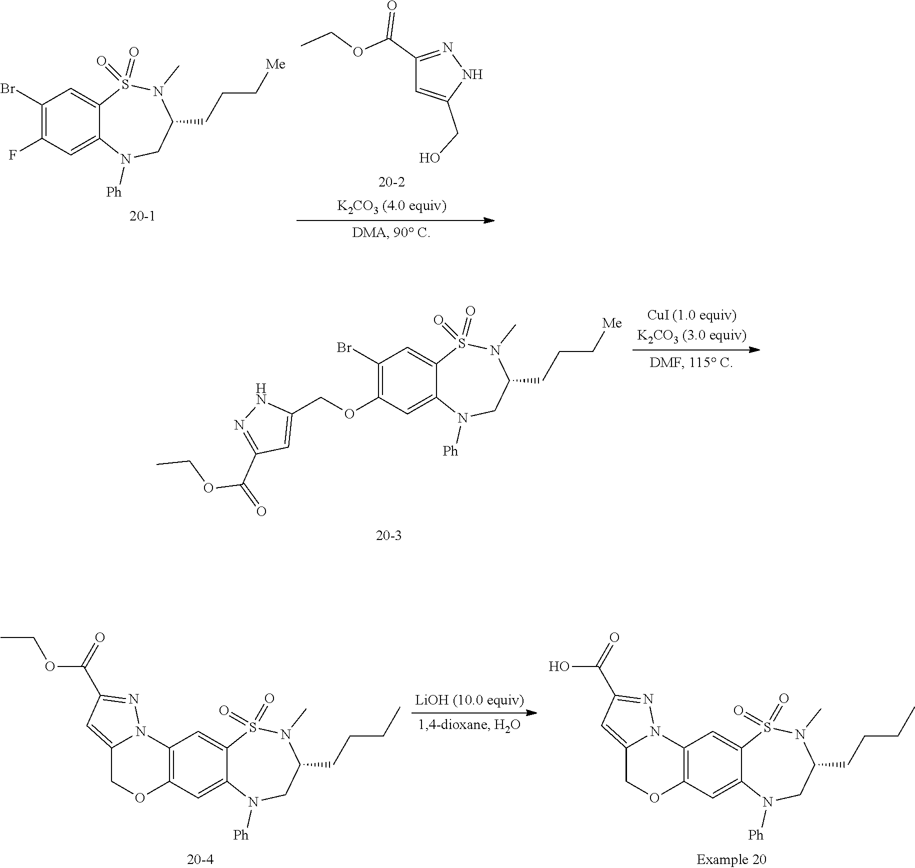

### Step 20-1

In a 1 dram vial equipped with a stir bar, (R)-8-bromo-3-butyl-7-fluoro-2-methyl-5-phenyl-2,3,4,5-tetrahydrobenzo[f][1,2,5]thiadiazepine 1,1-dioxide (100.0 mg, 0.23 mmol, 1.0 equiv), ethyl 5-(hydroxymethyl)-1H-pyrazole-3-carboxylate (77.0 mg, 0.45 mmol, 2.0 equiv, CAS #61453-48-3), and potassium carbonate (125.0 mg, 0.91 mmol, 4.0 equiv) were combined neat. Next, N,N-dimethylacetamide (1.5 mL, 0.15M) was added, and the resulting mixture was heated at 90° C. for 18 h. Upon cooling to room temperature, formic acid (0.5 mL) was added. The reaction mixture was passed through a 0.45 micron syringe filter and purified by RPHPLC to afford ethyl (R)-5-(((8-bromo-3-butyl-2-methyl-1,1-dioxido-5-phenyl-2,3,4,5-tetrahydrobenzo[f][1,2,5]thiadiazepin-7-yl)oxy)methyl)-1H-pyrazole-3-carboxylate (60.7 mg, 45% yield). ESI MS m/z=593.2 [M+H]⁺.

### Step 20-2

In a 20 mL vial equipped with a stir bar, (R)-5-(((8-bromo-3-butyl-2-methyl-1,1-dioxido-5-phenyl-2,3,4,5-tetrahydrobenzo[f][1,2,5]thiadiazepin-7-yl)oxy)methyl)-1H-pyrazole-3-carboxylate (60.7 mg, 0.10 mmol, 1.0 equiv) was dissolved in N,N-dimethylformamide (2.05 mL, 0.05M) under a nitrogen atmosphere. Next, potassium carbonate (42.5 mg, 0.31 mmol, 3.0 equiv), and copper(I) iodide (19.5 mg, 0.10 mmol, 1.0 equiv) were added, and the reaction mixture was heated at 115° C. for 18 h. Upon cooling to room temperature, the reaction mixture was treated with formic acid (0.5 mL) and passed through a 0.45 micron syringe filter. Purification by RPHPLC afforded ethyl (R)-9-butyl-10-methyl-7-phenyl-7,8,9,10-tetrahydro-4H-pyrazolo[1″,5″:4′,5′][1,4]oxazino[3′,2′:4,5]benzo[1,2-f][1,2,5]thiadiazepine-2-carboxylate 11,11-dioxide (21.4 mg, 41% yield). ESI MS m/z=511.2 [M+H]⁺.

### Step 20-3

In a 20 mL vial equipped with a stir bar, ethyl (R)-9-butyl-10-methyl-7-phenyl-7,8,9,10-tetrahydro-4H-pyrazolo[1″,5″:4′,5′][1,4]oxazino[3′,2′:4,5]benzo[1,2-f][1,2,5]thiadiazepine-2-carboxylate 11,11-dioxide (21.4 mg, 0.04 mmol, 1.0 equiv) was dissolved in a mixture of 1,4-dioxane (1.9 mL) and water (0.48 mL). Lithium hydroxide (10.0 mg, 0.42 mmol, 10.0 equiv) was added and the mixture was stirred at room temperature for 3 h. Formic acid (0.5 mL) was added, and the reaction mixture was concentrated, re-dissolved in DMF, passed through a 0.45 micron syringe filter, and purified by RPHPLC to afford (R)-9-butyl-10-methyl-7-phenyl-7,8,9,10-tetrahydro-4H-pyrazolo[1l″,5″:4′,5′][1,4]oxazino[3′,2′:4,5]benzo[1,2-f][1,2,5]thiadiazepine-2-carboxylic acid 11,11-dioxide. ESI MS m/z=483.2 [M+H]⁺.

### Ex. 21: Synthesis of (5R,10R)-10-butyl-11-methyl-8-phenyl-5-(trifluoromethyl)-8,9,10,11-tetrahydro-5H-benzo[3,4]chromeno[7,6-f][1,2,5]thiadiazepine-2-carboxylic acid 12,12-dioxide and (5S,10R)-10-butyl-11-methyl-8-phenyl-5-(trifluoromethyl)-8,9,10,11-tetrahydro-5H-benzo[3,4]chromeno[7,6-f][1,2,5]thiadiazepine-2-carboxylic acid 12,12-dioxide

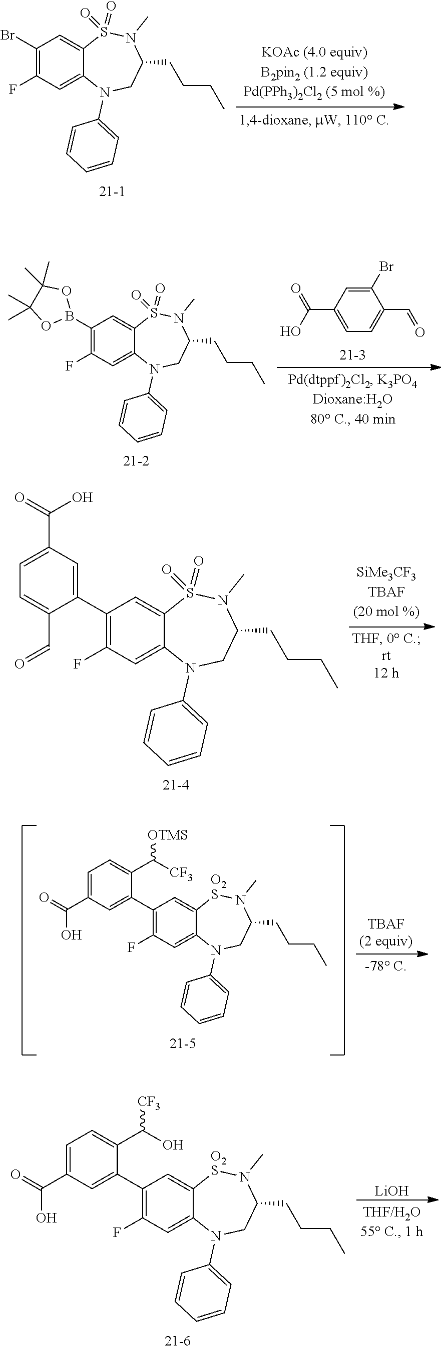

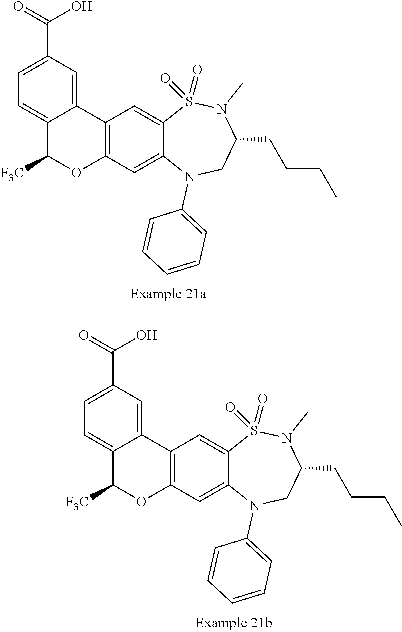 [SMILES: CCCC[C@@H]1CN(c2ccccc2)c2cc3c(cc2S(=O)(=O)N1C)-c1cc(C(=O)O)ccc1[C@H](C(F)(F)F)O3 ; CCCC[C@@H]1CN(c2ccccc2)c2cc3c(cc2S(=O)(=O)N1C)-c1cc(C(=O)O)ccc1[C@H](C(F)(F)F)O3]

### Step 21-1

In a 25 mL microwave reaction vial, (R)-8-bromo-3-butyl-7-fluoro-2-methyl-5-phenyl-2,3,4,5-tetrahydrobenzo[f][1,2,5]thiadiazepine 1,1-dioxide (800 mg), potassium acetate (711.5 mg, 4.0 equiv), bis(triphenylphosphine)palladium(II) dichloride (63.6 mg, 5 mol % equiv, CAS #: 13965-03-2), and bis(pinacolato)diboron (552.3 mg, 1.20 equiv, CAS #: 73183-34-3) were combined neat. The vessel was flushed with nitrogen three times and dry 1,4-dioxane (18.1 mL) was added. Then, the reaction was heated at 110° C. in microwave reactor for 40 minutes. The reaction mixture was then filtered through celite and concentrated. Purification of the residue by silica gel column chromatography afforded (R)-3-butyl-7-fluoro-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (712.0 mg, 80% yield). ESI MS m/z=489.2 [M+H]⁺.

### Step 21-2

In a 20 mL reaction vial equipped with a stir bar, (R)-3-butyl-7-fluoro-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (200.0 mg), 3-bromo-4-formyl-benzoic acid (187.6 mg, 2.0 equiv, CAS #91760-66-6), [1,1′-bis(di-tert-butylphosphino)ferrocene]dichloropalladium(II) (26.7 mg, 0.1 equiv, CAS #95408-45-0) and potassium phosphate tribasic (260.8 mg, 3.0 equiv, CAS #: 7778-53-2) were combined. The reaction vial was flushed with nitrogen three times and 1,4-dioxane (3.3 mL) and water (0.8 mL) were added. The reaction mixture was heated at 80° C. for 1 h on a heating block. Upon cooling to the room temperature, reaction mixture was quenched with formic acid (1 mL) and concentrated. Purification of the residue by silica gel column chromatography to afforded (R)-3-(3-butyl-7-fluoro-2-methyl-1,1-dioxido-5-phenyl-2,3,4,5-tetrahydrobenzo[f][1,2,5]thiadiazepin-8-yl)-4-formylbenzoic acid (152 mg, 72% yield). ESI MS m/z=511.1 [M+H]⁺.

### Step 21-3

In a 2 dram vial equipped with a stir bar, (R)-3-(3-butyl-7-fluoro-2-methyl-1,1-dioxido-5-phenyl-2,3,4,5-tetrahydrobenzo[f][1,2,5]thiadiazepin-8-yl)-4-formylbenzoic acid (20.0 mg) was dissolved in dry tetrahydrofuran (0.4 mL) under a nitrogen atmosphere. (Trifluoromethyl)trimethylsilane (0.0094 mL, 1.5 equiv, CAS #: 81290-20-2) and tetrabutylammonium fluoride (1.0 M in tetrahydrofuran, 0.0078 mL, 0.2 equiv, CAS #: 429-41-4) were added dropwise at 0° C. After complete addition, the ice bath was removed, and reaction mixture was stirred for 12 h at room temperature. Additional tetrabutylammonium fluoride (1.0 M in tetrahydrofuran, 0.078 mL, 2.0 equiv) was added at −78° C. and the mixture was stirred for 30 min at the same temperature. Reaction progress was monitored by LC-MS. The crude reaction mixture was quenched by addition of saturated aqueous ammonium chloride solution and extracted with ethyl acetate three times. The combined organic layers were washed with water and brine, dried over sodium sulfate, and concentrated to afford 3-((R)-3-butyl-7-fluoro-2-methyl-1,1-dioxido-5-phenyl-2,3,4,5-tetrahydrobenzo[f][1,2,5]thiadiazepin-8-yl)-4-(2,2,2-trifluoro-1-hydroxyethyl)benzoic acid, 21-6, as a mixture of diastereomers. The crude diastereomeric mixture was transferred to the next step without further purification. ESI MS m/z=581.2 [M+H]⁺.

### Step 21-4

Crude 3-((R)-3-butyl-7-fluoro-2-methyl-1,1-dioxido-5-phenyl-2,3,4,5-tetrahydrobenzo[f][1,2,5]thiadiazepin-8-yl)-4-(2,2,2-trifluoro-1-hydroxyethyl)benzoic acid, 21-6, as a mixture of diastereomers was dissolved in tetrahydrofuran (1 mL) and lithium hydroxide (1 N solution in water, 0.78 mL, 20.0 equiv) was added to the resulting solution. The reaction mixture was stirred for 1 h at 55° C. After complete conversion, the reaction mixture was quenched with 1N HCl and extracted three times with ethyl acetate. The combined organic layers were washed with water and brine and dried over sodium sulfate. The crude residue was re-dissolved in 2.0 mL of dimethyl sulfoxide, passed through a 0.45 μm syringe filter, and purified by RPHPLC to afford (5R,10R)-10-butyl-11-methyl-8-phenyl-5-(trifluoromethyl)-8,9,10,11-tetrahydro-5H-benzo[3,4]chromeno[7,6-f][1,2,5]thiadiazepine-2-carboxylic acid 12,12-dioxide (5.1 mg, 23% yield) and (5S,10R)-10-butyl-11-methyl-8-phenyl-5-(trifluoromethyl)-8,9,10,11-tetrahydro-5H-benzo[3,4]chromeno[7,6-f][1,2,5]thiadiazepine-2-carboxylic acid 12,12-dioxide (3.9 mg, 18% yield. ESI MS m/z=561.2 [M+H]⁺.

### Ex22: Synthesis of (5S,10R)-5-(tert-butyl)-10-butyl-11-methyl-8-phenyl-8,9,10,11-tetrahydro-5H-benzo[3,4]chromeno[7,6-f][1,2,5]thiadiazepine-2-carboxylic acid 12,12-dioxide and (5R,10R)-5-(tert-butyl)-10-butyl-11-methyl-8-phenyl-8,9,10,11-tetrahydro-5H-benzo[3,4]chromeno[7,6-f][1,2,5]thiadiazepine-2-carboxylic acid 12,12-dioxide

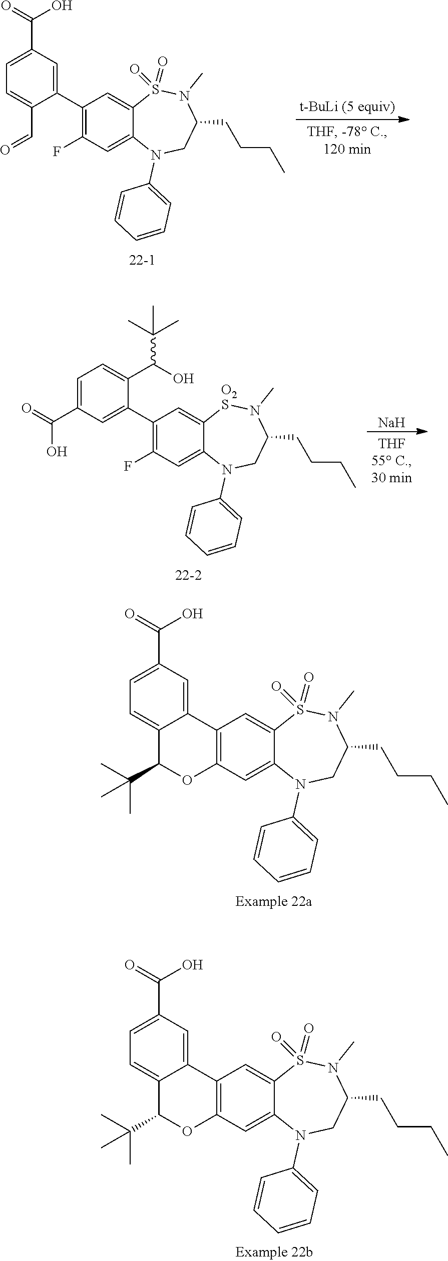

### Step 22-1

In a 2 dram vial equipped with a stir bar, (R)-3-(3-butyl-7-fluoro-2-methyl-1,1-dioxido-5-phenyl-2,3,4,5-tetrahydrobenzo[f][1,2,5]thiadiazepin-8-yl)-4-formylbenzoic acid (25.0 mg,) was dissolved in dry tetrahydrofuran (0.4 mL). The resulting mixture was cooled to −78° C. and tert-butyllithium solution (1.7 M in pentane, 0.07 mL, 2.5 equiv) was added dropwise. The reaction mixture was stirred for 30 minutes at −78° C. Additional tert-butyllithium solution (0.07 mL, 2.5 equiv) was added and continued stirring for additional 90 minutes. After this time, the reaction mixture was carefully quenched with 1N HCl and the aqueous phase was extracted three times with ethyl acetate. The combined organic layers were washed with water and brine, dried over sodium sulfate, and concentrated to afford 3-((R)-3-butyl-7-fluoro-2-methyl-1,1-dioxido-5-phenyl-2,3,4,5-tetrahydrobenzo[f][1,2,5]thiadiazepin-8-yl)-4-(1-hydroxy-2,2-dimethylpropyl)benzoic acid as a mixture of diastereomers that was used in the next step without further purification. ESI MS m/z=567.0 [M−H]−.

### Step 22-2

The crude 3-((R)-3-butyl-7-fluoro-2-methyl-1,1-dioxido-5-phenyl-2,3,4,5-tetrahydrobenzo[f][1,2,5]thiadiazepin-8-yl)-4-(1-hydroxy-2,2-dimethylpropyl)benzoic acid (27.9 mg) was dissolved in dry tetrahydrofuran (1.0 mL) under nitrogen atmosphere, followed by addition of sodium hydride (60% in mineral oil, 5.9 mg, 3.0 equiv). Then, the reaction mixture was stirred at 55° C. for 30 minutes. After complete conversion, the reaction mixture was quenched with 1N HCl and the aqueous phase extracted three times with ethyl acetate. The combined organic layers were washed with water and brine and dried over sodium sulfate. Upon concentration, the crude residue was re-dissolved in 2.0 mL of dimethyl sulfoxide, passed through a 0.45 μm syringe filter, and purified by RPHPLC to afford (5S,10R)-5-(tert-butyl)-10-butyl-11-methyl-8-phenyl-8,9,10,11-tetrahydro-5H-benzo[3,4]chromeno[7,6-f][1,2,5]thiadiazepine-2-carboxylic acid 12,12-dioxide (4.1 mg, 15% yield) and (5R,10R)-5-(tert-butyl)-10-butyl-11-methyl-8-phenyl-8,9,10,11-tetrahydro-5H-benzo[3,4]chromeno[7,6-f][1,2,5]thiadiazepine-2-carboxylic acid 12,12-dioxide (2.2 mg, 8.2% yield). ESI MS m/z=549.2 [M+H]⁺.

### Ex. 23: Synthesis of (R)-10-butyl-5,5,11-trimethyl-8-phenyl-8,9,10,11-tetrahydro-5H-benzo[3,4]chromeno[7,6-f][1,2,5]thiadiazepine-2-carboxylic acid 12,12-dioxide

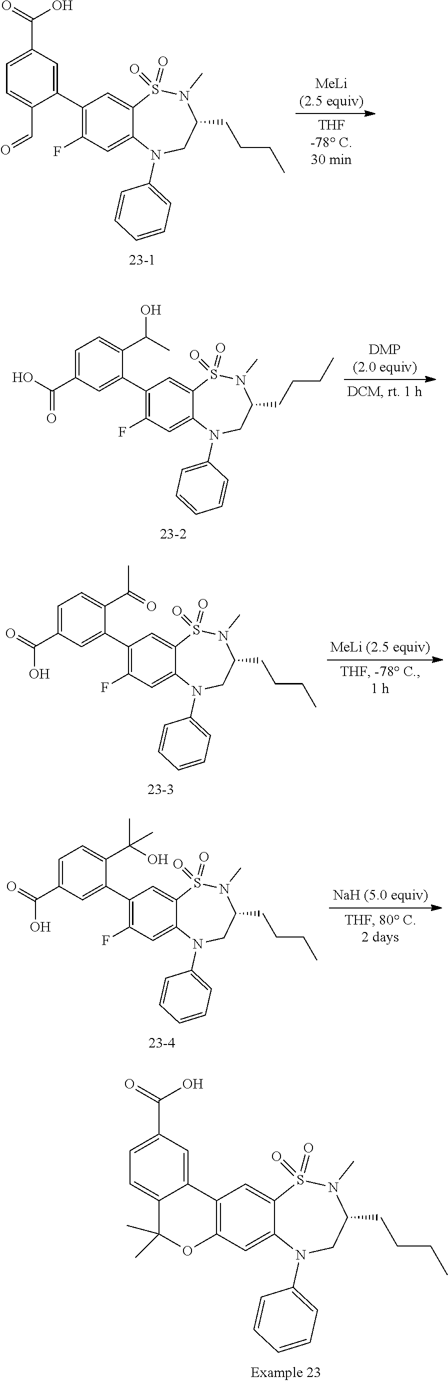

### Step 23-1

In a 2 dram vial equipped with a stir bar, (R)-3-(3-butyl-7-fluoro-2-methyl-1,1-dioxido-5-phenyl-2,3,4,5-tetrahydrobenzo[f][1,2,5]thiadiazepin-8-yl)-4-formylbenzoic acid (114 mg) was dissolved in THF (2.2 mL) under a nitrogen atmosphere. The resulting solution was cooled to −78° C. and methyl lithium (2.5 M in diethyl ether, 372 μl, 2.5 equiv) was added dropwise. The resulting mixture was stirred for 30 minutes at −78° C. Reaction progress was monitored by LC-MS. After complete conversion, the reaction mixture was quenched with 1N HCl and extracted with ethyl acetate three times. The combined organic layers were washed with water and brine, dried over sodium sulfate and concentrated to afford 3-((R)-3-butyl-7-fluoro-2-methyl-1,1-dioxido-5-phenyl-2,3,4,5-tetrahydrobenzo[f][1,2,5]thiadiazepin-8-yl)-4-((S)-1-hydroxyethyl)benzoic acid as a diastereomeric mixture that was used in the next step without purification. ESI MS m/z=525.2 [M−H]−.

### Step 23-2

In a 2 dram vial equipped with a stir bar, 3-((R)-3-butyl-7-fluoro-2-methyl-1,1-dioxido-5-phenyl-2,3,4,5-tetrahydrobenzo[f][1,2,5]thiadiazepin-8-yl)-4-(1-hydroxyethyl)benzoic acid (0.12 g) was dissolved in dry dichloromethane (2.2 mL). The resulting solution was cooled to 0° C. and Dess-Martin periodinane (0.19 g, 2.2 equiv.) was added in one portion. Then, the reaction mixture was stirred at room temperature for 1 h. The reaction mixture was concentrated and purified by reversed-phase flash chromatography to afford (R)-4-acetyl-3-(3-butyl-7-fluoro-2-methyl-1,1-dioxido-5-phenyl-2,3,4,5-tetrahydrobenzo[f][1,2,5]thiadiazepin-8-yl)benzoic acid (86 mg, 74% yield). ESI MS m/z=525.3 [M+H]⁺.

### Step 23-3

In a 2 dram vial, (R)-4-acetyl-3-(3-butyl-7-fluoro-2-methyl-1,1-dioxido-5-phenyl-2,3,4,5-tetrahydrobenzo[f][1,2,5]thiadiazepin-8-yl)benzoic acid (86 mg) was dissolved in dry tetrahydrofuran (1.6 mL) under a nitrogen atmosphere. The resulting solution was cooled to −78° C. and methyl lithium (1.5 M in diethyl ether, 273 μl, 2.5 equiv) was added dropwise. The resulting mixture was stirred for 60 minutes at −78° C. Reaction progress was monitored by LC-MS. After complete conversion, the reaction mixture was quenched with 1N HCl and extracted with ethyl acetate three times. The combined organic layers was washed with water and brine, dried over sodium sulfate and concentrated to afford (R)-3-(3-butyl-7-fluoro-2-methyl-1,1-dioxido-5-phenyl-2,3,4,5-tetrahydrobenzo[f][1,2,5]thiadiazepin-8-yl)-4-(2-hydroxypropan-2-yl)benzoic acid that was used in the next step without further purification. ESI MS m/z=539.2. [M−H]⁻.

### Step 23-4

In a 2 dram vial, (R)-3-(3-butyl-7-fluoro-2-methyl-1,1-dioxido-5-phenyl-2,3,4,5-tetrahydrobenzo[f][1,2,5]thiadiazepin-8-yl)-4-(2-hydroxypropan-2-yl)benzoic acid (89.0 mg) was dissolved in dry tetrahydrofuran (1.6 mL) under a nitrogen atmosphere. Sodium hydride (60% in mineral oil, 33.0 mg, 5.0 equiv) was added at room temperature. The reaction mixture was then stirred for 2 days at 80° C. Reaction progress was monitored by LC-MS. Upon complete consumption of the starting material, the reaction mixture was quenched with 1N HCl and extracted with ethyl acetate three times. The combined organic layers were washed with water and brine, dried over sodium sulfate and concentrated. The crude residue was re-dissolved in 2.0 mL of dimethyl sulfoxide, passed through a 0.45 μm syringe filter, and purified by RPHPLC to afford (R)-10-butyl-5,5,11-trimethyl-8-phenyl-8,9,10,11-tetrahydro-5H-benzo[3,4]chromeno[7,6-f][1,2,5]thiadiazepine-2-carboxylic acid 12,12-dioxide (38 mg, 44% yield). ESI MS m/z=521.3 [M+H]⁺. ¹H NMR (400 MHz, dimethylsulfoxide-d₆) δ 13.27 (br, 1H), 8.32-8.22 (m, 2H), 8.25 (s, 1H), 7.95 (dd, J=8.1, 1.6 Hz, 1H), 7.56 (d, J=8.1 Hz, 1H), 7.24 (dd, J=8.6, 7.2 Hz, 2H), 6.92-6.82 (m, 3H), 6.68 (s, 1H), 4.05 (d, J=15.9 Hz, 1H), 3.85-3.65 (m, 1H), 3.58-3.41 (m, 1H), 2.61 (s, 3H), 1.65 (s, 3H), 1.60 (s, 3H), 1.60-1.50 (m, 2H), 1.44-1.24 (m, 4H), 0.90 (t, J=6.9 Hz, 3H).

### Ex. 24: Synthesis of 10-butyl-11-methyl-8-phenyl-8,9,10,11-tetrahydro-6H-benzo[3,4]isochromeno[7,6-f][1,2,5]thiadiazepine-2-carboxylic acid 12,12-dioxide

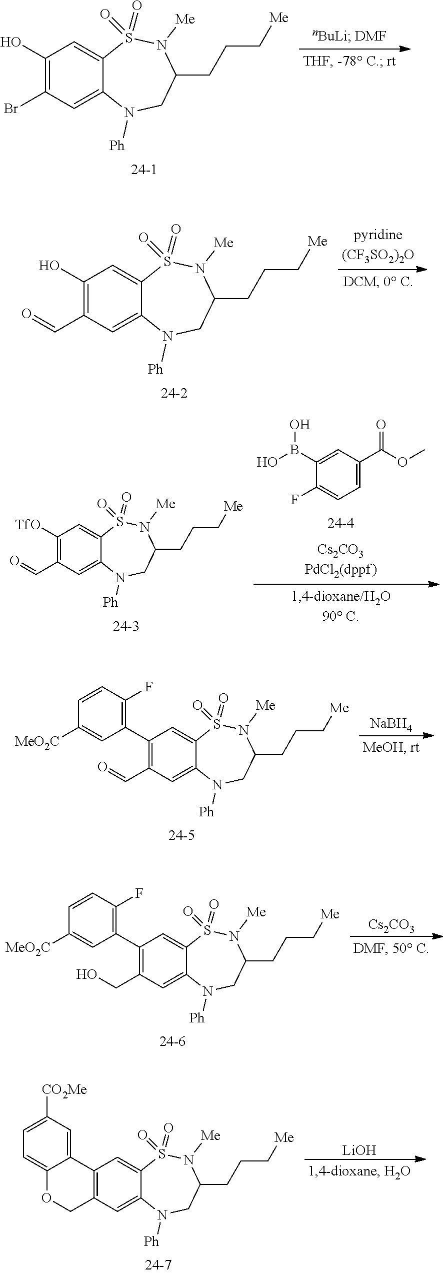

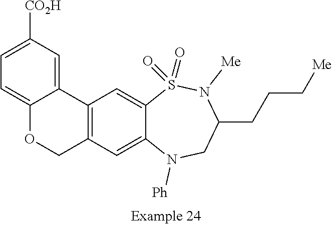 [SMILES: CCCCC1CN(c2ccccc2)c2cc3c(cc2S(=O)(=O)N1C)-c1cc(C(=O)O)ccc1OC3]

### Step 24-1

To a mixture of 7-bromo-3-butyl-8-hydroxy-2-methyl-5-phenyl-2,3,4,5-tetrahydrobenzo[f][1,2,5]thiadiazepine 1,1-dioxide (5.00 g, 11.4 mmol) in THF (37.9 ml) that had been cooled to −78° C. was added n-BuLi (12.19 ml, 17.1 mmol) dropwise. The reaction was stirred for 2 hours, then DMF (4.41 ml, 56.9 mmol) was added dropwise at −78° C., and the solution was warmed to room temperature overnight. The reaction was quenched with saturated aqueous NH₄Cl and extracted with EtOAc. The combined organic layers were washed with brine (100 mL), dried over Na₂SO₄, and concentrated. The residue was purified by column chromatography (5 to 15% EtOAc in hexanes) to provide 3-butyl-8-hydroxy-2-methyl-5-phenyl-2,3,4,5-tetrahydrobenzo[f][1,2,5]thiadiazepine-7-carbaldehyde 1,1-dioxide (2 g, 5.15 mmol, 45% yield). ESI MS m/z=389.3 [M+H]⁺.

### Step 24-2

To a flask containing 3-butyl-8-hydroxy-2-methyl-5-phenyl-2,3,4,5-tetrahydrobenzo[f][1,2,5]thiadiazepine-7-carbaldehyde 1,1-dioxide (2 g, 5.15 mmol) in dry CH₂Cl₂ (25.7 ml) was added analytical-grade pyridine (0.583 ml, 7.21 mmol) under N₂. The solution was cooled to 0° C., then treated with dropwise addition of triflicanhydride (1.305 ml, 7.72 mmol) and allowed to stir at 0° C. for 2 hours. The reaction was then diluted with CH₂Cl₂, quenched with saturated NaHCO₃, and extracted with CH₂Cl₂. The combined organic layers were washed with brine, dried over anhydrous Na₂SO₄, and concentrated. The residue was purified by column chromatography (5 to 15% EtOAc in hexanes) to provide rac-3-butyl-7-formyl-2-methyl-1,1-dioxido-5-phenyl-2,3,4,5-tetrahydrobenzo[f][1,2,5]thiadiazepin-8-yl trifluoromethanesulfonate (1.16 g, 2.219 mmol, 43% yield). ESI MS m/z=521.2 [M+H]⁺.

### Step 24-3

To a vial containing 3-butyl-7-formyl-2-methyl-1,1-dioxido-5-phenyl-2,3,4,5-tetrahydrobenzo[f][1,2,5]thiadiazepin-8-yl trifluoromethanesulfonate (200.0 mg, 0.384 mmol), (2-fluoro-5-(methoxycarbonyl)phenyl)boronic acid (95.0 mg, 0.480 mmol), cesium carbonate (376 mg, 1.153 mmol), and PdCl₂(dppf) (28.1 mg, 0.038 mmol) was added 1,4-dioxane (3.34 ml) and water (0.501 ml) under N₂. The reaction was stirred and heated at 90° C. for 4 hours. The reaction was then cooled to room temperature and quenched with HCl. The aqueous phase was extracted with EtOAc. The organic layer was dried, concentrated, then purified by column chromatography (0-25% ethyl acetate/cyclohexane) to provide ethyl methyl 3-(3-butyl-7-formyl-2-methyl-1,1-dioxido-5-phenyl-2,3,4,5-tetrahydrobenzo[f][1,2,5]thiadiazepin-8-yl)-4-fluorobenzoate (124 mg, 0.236 mmol, 62% yield). ESI MS m/z=525.2 [M+H]⁺.

### Step 24-4

To methyl 3-(3-butyl-7-formyl-2-methyl-1,1-dioxido-5-phenyl-2,3,4,5-tetrahydrobenzo[f][1,2,5]thiadiazepin-8-yl)-4-fluorobenzoate (75.0 mg, 0.143 mmol) in MeOH (1.430 ml), was added NaBH₄ (10.8 mg, 0.286 mmol). The reaction mixture was stirred at room temperature for 1 h, then 1N HCl was added and the reaction was extracted with EtOAc. The organic layer was dried over Na₂SO₄, filtered, and concentrated. The crude product methyl 3-(3-butyl-7-(hydroxymethyl)-2-methyl-1,1-dioxido-5-phenyl-2,3,4,5-tetrahydrobenzo[f][1,2,5]thiadiazepin-8-yl)-4-fluorobenzoate (74.0 mg, 0.141 mmol, 98% yield) was used without further purification. ESI MS m/z=527.2 [M+H]⁺.

### Step 24-5

A solution of methyl 3-(3-butyl-7-(hydroxymethyl)-2-methyl-1,1-dioxido-5-phenyl-2,3,4,5-tetrahydrobenzo[f][1,2,5]thiadiazepin-8-yl)-4-fluorobenzoate (25.0 mg, 0.047 mmol) and Cs₂CO₃ (77.0 mg, 0.237 mmol) in DMF (0.475 ml) was heated to 50° C. Upon complete conversion, the reaction was diluted with EtOAc, washed once with 1N HCl, three times with water, and once with brine. The organic layer was dried over Na₂SO₄, filtered, and concentrated to provide methyl 10-butyl-11-methyl-8-phenyl-8,9,10,11-tetrahydro-6H-benzo[3,4]isochromeno[7,6-f][1,2,5]thiadiazepine-2-carboxylate 12,12-dioxide (22.0 mg, 0.043 mmol, 91% yield), which was used without further purification in subsequent step. [M+H] m/z 507.200.

### Step 24-6

To methyl 10-butyl-11-methyl-8-phenyl-8,9,10,11-tetrahydro-6H-benzo[3,4]isochromeno[7,6-f][1,2,5]thiadiazepine-2-carboxylate 12,12-dioxide (26 mg, 0.051 mmol) in dioxane (0.467 ml) and H₂O (0.047 mL) was added LiOH (2.458 mg, 0.103 mmol). The reaction was stirred at room temperature. Upon complete conversion, formic acid (50 L) was added and the mixture was subjected to preparative RPHPLC purification to provide 10-butyl-11-methyl-8-phenyl-8,9,10,11-tetrahydro-6H-benzo[3,4]isochromeno[7,6-f][1,2,5]thiadiazepine-2-carboxylic acid 12,12-dioxide (3 mg, 6.09 μmol, 11.87% yield). ¹H NMR (400 MHz, DMSO) δ 13.02 (s, 1H), 8.39 (d, J=2.1 Hz, 1H), 8.19 (s, 1H), 7.89 (dd, J=8.5, 2.0 Hz, 1H), 7.33 (s, 1H), 7.23 (t, J=7.1 Hz, 2H), 7.11 (d, J=8.5 Hz, 1H), 6.87-6.79 (m, 3H), 5.26 (dd, J=22.7, 14.4 Hz, 2H), 4.14 (d, J=16.1 Hz, 1H), 3.82-3.77 (m, 1H), 3.43-3.38 (m, 1H), 2.58 (s, 3H), 1.60-1.56 (m, 2H), 1.43-1.28 (m, 4H), 0.91 (t, J=6.9 Hz, 3H). ESI MS m/z=492.3 [M+H]⁺.

### Ex. 25: Synthesis (R)-10-cyclohexyl-11-methyl-8,9,10,11-tetrahydro-5H-benzo[3,4]chromeno[7,6-f][1,2,5]thiadiazepine-2-carboxylic acid 12,12-dioxide

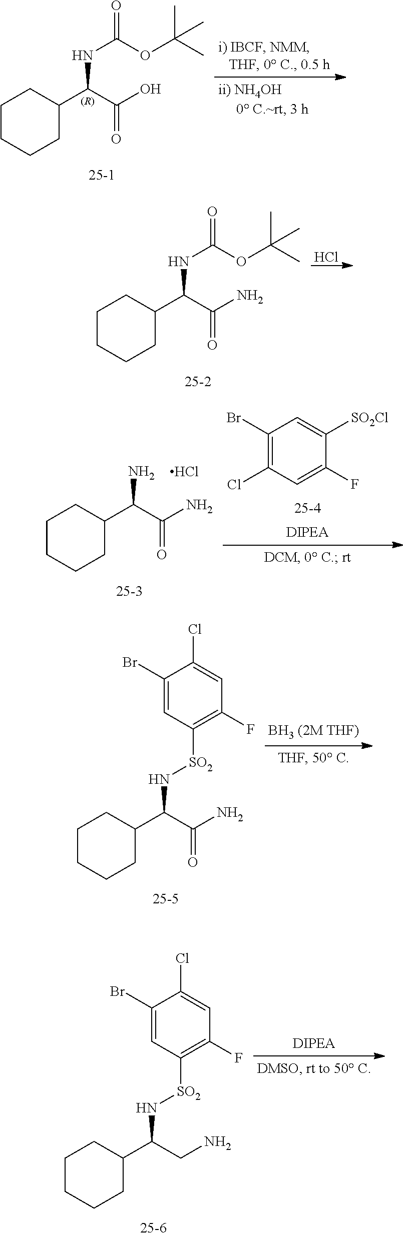

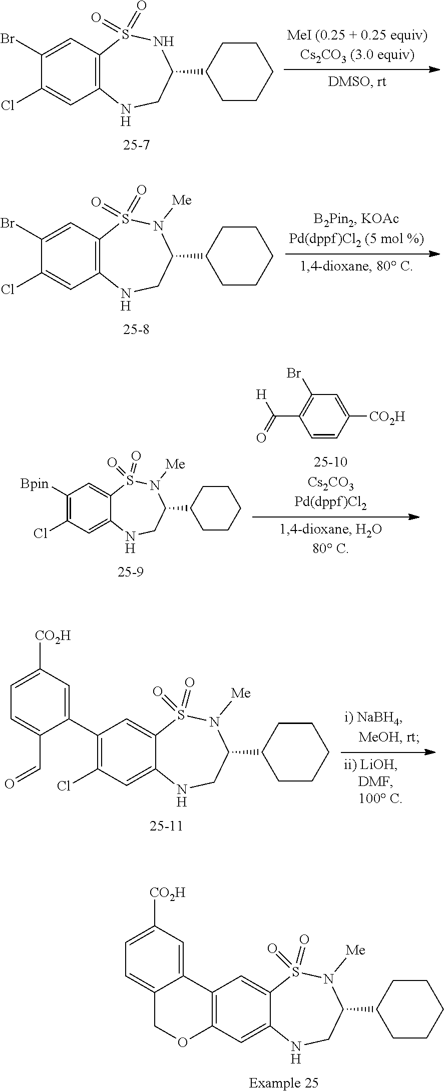

### Step 25-1

To a solution of compound (R)-2-((tert-butoxycarbonyl)amino)-2-cyclohexylacetic acid (60.0 g, 233.2 mmol, 1.0 eq, CAS #70491-05-3) in THF (600 mL) was added IBCF (38.2 g, 279.8 mmol, 1.2 eq) and NMM (28.3 g, 279.8 mmol, 1.2 eq) dropwise at 0° C. Then the reaction mixture was stirred at 0° C. for 0.5 h. After this time, to the reaction mixture was added NH₄OH (24.4 g, 279.8 mmol, 1.2 eq) and the resulting mixture was stirred for 3 h at room temperature. The mixture was filtered to give compound tert-butyl (R)-(2-amino-1-cyclohexyl-2-oxoethyl)carbamate (50.0 g, 83%) as a white solid, which was used in the next step without any further purification. ¹HNMR (400 MHz, DMSO-d₆) δ ppm 7.26 (s, 1H), 6.98 (s, 1H), 6.49 (d, J=9.0 Hz, 1H), 3.74 (d, J=7.2 Hz, 1H), 1.65-1.53 (m, 6H), 1.37 (s, 9H), 1.17-0.92 (m, 5H). ESI MS m/z=201.0 [M-56+H]⁺.

### Step 25-2

HCl in 1,4-dioxane (4M, 500 mL) was added to (R)-(2-amino-1-cyclohexyl-2-oxoethyl)carbamate (50.0 g, 195.0 mmol, 1.0 eq). The reaction mixture was stirred at room temperature for 4 h. After completion, the reaction mixture was concentrated to give compound (R)-2-amino-2-cyclohexylacetamide hydrochloride (35.0 g, crude) as a white solid, which was used in the next step without any further purification. ESI MS m/z=157.2 [M+H]⁺.

### Step 25-3

To a solution of compound (R)-2-amino-2-cyclohexylacetamide hydrochloride (35.0 g, 182.2 mmol, 1.0 eq) in DCM (350 mL) was added DIEA (70.5 g, 546.6 mmol, 3.0 eq) and 5-bromo-4-chloro-2-fluorobenzenesulfonyl chloride (55.7 g, 182.2 mmol, 1.0 eq). Then the reaction mixture was stirred at room temperature overnight. After completion, the mixture was filtered and the filter cake was triturated with EtOAc to give (R)-2-((5-bromo-4-chloro-2-fluorophenyl)sulfonamido)-2-cyclohexylacetamide (50.0 g, 64%) as a white solid. ¹HNMR (400 MHz, DMSO-d₆) δ ppm 8.16 (brs, 1H), 8.02 (d, J=7.2 Hz, 1H), 7.91 (d, J=9.6 Hz, 1H), 7.31 (s, 1H), 6.99 (s, 1H), 3.57 (d, J=7.2 Hz, 1H), 1.68-1.47 (m, 6H), 1.23-1.08 (m, 5H). ESI MS m/z=428.8 [M+H]⁺.

### Step 25-4

To (R)-2-((5-bromo-4-chloro-2-fluorophenyl)sulfonamido)-2-cyclohexylacetamide (40.0 g, 93.5 mmol, 1.0 eq) was added BH₃ (374 mL, 2 M in THF, 8.0 eq) at room temperature. The reaction mixture was stirred at 50° C. for 12 h. After completion, the resulting mixture was cooled to 0° C., and H₂O (300 mL) was added. The mixture was then extracted with EtOAc three times (500 mL per extraction), washed with brine (500 mL), and dried over Na₂SO₄. Filtration and concentration of the filtrate in vacuo and trituration of the residue (Petroleum ether: EtOAc=5:1) gave (R)—N-(2-amino-1-cyclohexylethyl)-5-bromo-4-chloro-2-fluorobenzenesulfonamide (35.0 g, 91%) as a white solid. ESI MS m/z=412.9 [M+H]⁺.

### Step 25-5

In a 1 L round-bottomed flask equipped with a stir bar and a reflux condenser under a nitrogen atmosphere, (R)—N-(2-amino-1-cyclohexylethyl)-5-bromo-4-chloro-2-fluorobenzenesulfonamide (12.55 g, 30.3 mmol, 1.0 equiv) was dissolved in DMSO (121 mL). Next, N,N-diisopropylethylamine (19.6 g, 26.5 mL, 5.0 equiv) was added and the resulting mixture was heated at 50° C. for 2.5 h. At this time, LCMS analysis of the crude reaction indicated clean conversion to the desired cyclization product. Upon cooling to room temperature, the reaction mixture was diluted with water and the aqueous layer was extracted with ethyl acetate. The combined organic layers were washed with brine and dried over sodium sulfate. Concentration afforded the crude (R)-8-bromo-7-chloro-3-cyclohexyl-2,3,4,5-tetrahydrobenzo[f][1,2,5]thiadiazepine 1,1-dioxide which was used in the subsequent step without purification. ESI MS m/z=393.0 [M+H]⁺.

### Step 25-6

In a 500 mL round-bottomed flask equipped with a stir bar and a reflux condenser the crude (R)-8-bromo-7-chloro-3-cyclohexyl-2,3,4,5-tetrahydrobenzo[f][1,2,5]thiadiazepine 1,1-dioxide formed above was dissolved in DMSO (100 mL) under a nitrogen atmosphere. Next, cesium carbonate (30.7 g, 94.0 mmol, 3.0 equiv) was added, followed by methyl iodide (1.12 g, 7.85 mmol, 0.25 equiv). After 15 min approximately 50% of the starting (R)-8-bromo-7-chloro-3-cyclohexyl-2,3,4,5-tetrahydrobenzo[f] [1,2,5]thiadiazepine 1,1-dioxide had been consumed based on LCMS analysis, and additional methyl iodide (1.12 g, 7.85 mmol, 0.25 equiv) was added. After an additional 15 min, all of the starting (R)-8-bromo-7-chloro-3-cyclohexyl-2,3,4,5-tetrahydrobenzo[f][1,2,5]thiadiazepine 1,1-dioxide had been cleanly converted to the desired N-Me sulfonamide. The reaction mixture was diluted with water, and the aqueous layer was extracted with ethyl acetate. The combined organic layers were washed with brine and dried over sodium sulfate. Upon concentration, the crude residue was purified by silica gel column chromatography to afford (R)-8-bromo-7-chloro-3-cyclohexyl-2-methyl-2,3,4,5-tetrahydrobenzo[f][1,2,5]thiadiazepine 1,1-dioxide as a white solid (5.17 g, 12.7 mmol, 40% yield). ESI MS m/z=407.0 [M+H]⁺.

### Step 25-7

A vial containing (R)-8-bromo-7-chloro-3-cyclohexyl-2-methyl-2,3,4,5-tetrahydrobenzo[f][1,2,5]thiadiazepine 1,1-dioxide (1.00 eq, 750 mg, 1.84 mmol), KOAc (4.00 eq, 722 mg, 7.36 mmol), Pd(dppf)C₁₂ (0.05 eq, 67 mg, 0.092 mmol), and B₂Pin₂ (1.20 eq, 561 mg, 2.21 mmol) in 1,4-dioxane (9.2 ml) was purged with N₂, then stirred at 80° C. for 10 h. The reaction was cooled, then filtered over celite using EtOAc to rinse. The solution was concentrated and the residue was purified by silica gel column chromatography (0-30% EtOAc/cyclohexane) to provide (R)-7-chloro-3-cyclohexyl-2-methyl-8-(4,4,5,5-tetramethyl-1,3,2-dioxaborolan-2-yl)-2,3,4,5-tetrahydrobenzo[f][1,2,5]thiadiazepine 1,1-dioxide (295.0 mg, 0.65 mmol, 35% yield). ESI MS m/z=455.1 [M+H]⁺.

### Step 25-8

To a vial containing 3-bromo-4-formylbenzoic acid (101.0 mg, 0.44 mmol), (R)-7-chloro-3-cyclohexyl-2-methyl-8-(4,4,5,5-tetramethyl-1,3,2-dioxaborolan-2-yl)-2,3,4,5-tetrahydrobenzo[f][1,2,5]thiadiazepine 1,1-dioxide (100.0 mg, 0.22 mmol), cesium carbonate (215.0 mg, 0.66 mmol), and PdCl₂(dppf) (16.0 mg, 0.022 mmol) was added 1,4-dioxane (0.5 mL) and water (0.1 mL) under N₂. The reaction was stirred and heated at 80° C. overnight. The reaction was then cooled to room temperature and quenched with HCl. The aqueous phase was extracted with EtOAc. The organic layer was dried, concentrated, then purified by silica gel column chromatography (0-25% ethyl acetate/cyclohexane) to provide (R)-3-(7-chloro-3-cyclohexyl-2-methyl-1,1-dioxido-2,3,4,5-tetrahydrobenzo[f][1,2,5]thiadiazepin-8-yl)-4-formylbenzoic acid (92.0 mg, 0.19 mmol, 88% yield). ESI MS m/z=477.1 [M+H]⁺.

### Step 25-9

To a solution of (R)-3-(7-chloro-3-cyclohexyl-2-methyl-1,1-dioxido-2,3,4,5-tetrahydrobenzo[f][1,2,5]thiadiazepin-8-yl)-4-formylbenzoic acid (25 mg, 0.052 mmol) in MeOH (0.26 mL) was added NaBH₄ (9.9 mg, 0.262 mmol). Upon complete conversion of the aldehyde to the corresponding alcohol, the reaction was diluted with DMF (0.52 mL) and LiOH (22.0 mg, 0.520 mmol) was added. The reaction was heated to 100° C. and stirred until complete conversion was observed. Formic acid (50 μL) was then added, and the mixture was subjected to preparative RPHPLC to afford (R)-10-cyclohexyl-11-methyl-8,9,10,11-tetrahydro-5H-benzo[3,4]chromeno[7,6-f][1,2,5]thiadiazepine-2-carboxylic acid 12,12-dioxide (10.0 mg, 0.023 mmol, 43% yield). ¹H NMR (400 MHz, DMSO) δ 13.17 (s, 1H), 8.11 (d, J=1.6 Hz, 1H), 7.91 (s, 1H), 7.81 (dd, J=7.8, 1.5 Hz, 1H), 7.35 (d, J=7.8 Hz, 1H), 6.91-6.86 (m, 1H), 6.44 (s, 1H), 5.20 (dd, J=20.4, 14.1 Hz, 2H), 3.87-3.83 (m, 1H), 3.43-3.34 (m, 1H), 3.17-3.13 (m, 1H), 2.80 (s, 3H), 2.10-1.99 (m, 1H), 1.67 (d, J=16.3 Hz, 4H), 1.17 (q, J=16.2 Hz, 4H), 0.99 (dt, J=24.2, 11.7 Hz, 2H). ESI MS m/z=443.2 [M+H]⁺.

### Ex. 26: Synthesis of (R)-3,4-difluoro-10-isobutyl-11-methyl-8,9,10,11-tetrahydro-5H-benzo[3,4]chromeno[7,6-f][1,2,5]thiadiazepine-2-carboxylic acid 12,12-dioxide

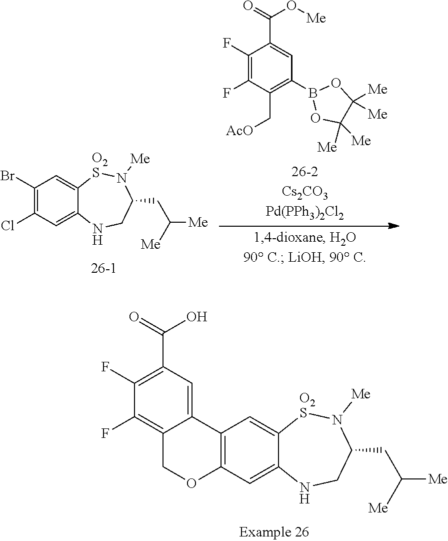 [SMILES: COC(=O)c1cc(B2OC(C)(C)C(C)(C)O2)c(COC(C)=O)c(F)c1F ; CC(C)C[C@@H]1CNc2cc(Cl)c(Br)cc2S(=O)(=O)N1C ; CC(C)C[C@@H]1CNc2cc3c(cc2S(=O)(=O)N1C)-c1cc(C(=O)O)c(F)c(F)c1CO3]

### Step 26-1

A solution of (R)-8-bromo-7-chloro-3-isobutyl-2-methyl-2,3,4,5-tetrahydrobenzo[f][1,2,5]thiadiazepine 1,1-dioxide (34.0 mg, 0.088 mmol), cesium carbonate (86.0 mg, 0.265 mmol), Pd(PPh₃)₂Cl₂ (6.21 mg, 8.84 μmol), and methyl 4-(acetoxymethyl)-2,3-difluoro-5-(4,4,5,5-tetramethyl-1,3,2-dioxaborolan-2-yl)benzoate (49.0 mg, 0.133 mmol) in 1,4-dioxane (0.769 mL) and H₂O (0.115 mL) under N₂ was heated at 90° C. for 2 hours. Upon complete conversion (monitored by LC-MS), LiOH (21.0 mg, 0.884 mmol) and H₂O (0.12 mL) was added and the reaction was stirred at 90° C. for 4 hours. The reaction was quenched with formic acid (50 μL) and subjected directly to preparative RPHPLC purification to provide (R)-3,4-difluoro-10-isobutyl-11-methyl-8,9,10,11-tetrahydro-5H-benzo[3,4]chromeno[7,6-f][1,2,5]thiadiazepine-2-carboxylic acid 12,12-dioxide (23.0 mg, 0.051 mmol, 58% yield). 1H NMR (400 MHz, DMSO) δ 13.77 (s, 1H), 7.93 (s, 1H), 7.86 (d, J=5.6 Hz, 1H), 6.84 (d, J=7.2 Hz, 1H), 6.54 (s, 1H), 5.31 (s, 2H), 3.70-3.63 (m, 1H), 3.56 (t, J=12.8 Hz, 1H), 3.21-3.12 (m, 1H), 2.72 (s, 3H), 1.77-1.65 (m, 1H), 1.65-1.54 (m, 1H), 1.30-1.18 (m, 1H), 0.94 (dd, J=6.6, 1.4 Hz, 6H). ESI MS m/z=453.2 [M+H]⁺.

### Ex. 27: Synthesis of (R)-10-cyclohexyl-6,11-dimethyl-8-phenyl-5,6,8,9,10,11-hexahydro-[1,2,5]thiadiazepino[7,6-b]phenanthridine-2-carboxylic acid 12,12-dioxide

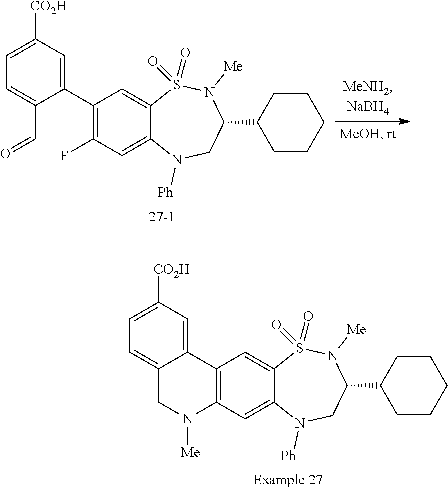 [SMILES: CN1[C@H](C2CCCCC2)CN(c2ccccc2)c2cc(F)c(-c3cc(C(=O)O)ccc3C=O)cc2S1(=O)=O ; CN1Cc2ccc(C(=O)O)cc2-c2cc3c(cc21)N(c1ccccc1)C[C@@H](C1CCCCC1)N(C)S3(=O)=O]

### Step 27-1

To a solution of (R)-3-(3-butyl-7-fluoro-2-methyl-1,1-dioxido-5-phenyl-2,3,4,5-tetrahydrobenzo[f][1,2,5]thiadiazepin-8-yl)-4-formylbenzoic acid (30.0 mg, 0.059 mmol) in MeOH (0.588 mL) was added 1 M methanamine in MeOH (58.8 μL, 0.118 mmol). The reaction was stirred for 2 h, then NaBH₄ (6.7 mg, 0.176 mmol) was added. The reaction mixture was stirred at room temperature for 1 h, then 2N HCl was added and the reaction mixture was extracted with EtOAc. The organic layer was dried over Na₂SO₄, filtered, and concentrated. The residue was then purified by preparative RPHPLC to afford (R)-10-butyl-6,11-dimethyl-8-phenyl-5,6,8,9,10,11-hexahydro-[1,2,5]thiadiazepino[7,6-b]phenanthridine-2-carboxylic acid 12,12-dioxide (8.0 mg, 0.016 mmol, 26.9% yield) as an off-white solid. ¹H NMR (400 MHz, DMSO) δ 13.19 (s, 1H), 8.24 (d, J=1.6 Hz, 1H), 8.08 (s, 1H), 7.87 (dd, J=7.9, 1.6 Hz, 1H), 7.36 (d, J=7.9 Hz, 1H), 7.18 (dd, J=8.6, 7.3 Hz, 2H), 6.79-6.71 (m, 3H), 6.60 (s, 1H), 4.50 (dd, J=21.3, 15.0 Hz, 2H), 4.04 (d, J=16.1 Hz, 1H), 3.86-3.81 (m, 1H), 3.25 (s, 1H), 2.84 (s, 3H), 2.52 (s, 3H), 1.64-1.48 (m, 2H), 1.38-1.32 (m, 4H), 0.91 (t, J=7.5 Hz, 3H). ESI MS m/z=506.2 [M+H]⁺.

The following examples were prepared using a procedure similar to that used for Ex. 27:

| Ex. | | | |
|---|---|---|---|
| # | Structure | MS | NMR |
| 28 | 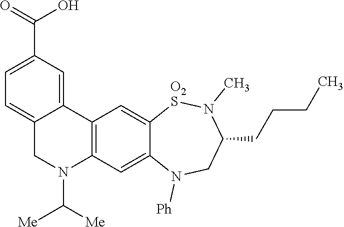 [SMILES: CCCC[C@@H]1CN(c2ccccc2)c2cc3c(cc2S(=O)(=O)N1C)-c1cc(C(=O)O)ccc1CN3C(C)C] | [M + H] m/z 534.412 | ¹H NMR (400 MHz, DMSO) δ 13.18 (s, 1H), 8.22 (d, J = 1.6 Hz, 1H), 8.11 (s, 1H), 7.88 (dd, J = 7.8, 1.6 Hz, 1H), 7.45 (d, J = 7.9 Hz, 1H), 7.19 (dd, J = 8.7, 7.1 Hz, 2H), 6.79-6.71 (m, 4H), 4.36 (s, 2H), 4.09-3.92 (m, 2H), 3.89-3.79 (m, 1H), 2.54 (s, 3H), 1.67-1.45 (m, 2H), 1.42- 1.31 (m, 5H), 1.17 (dd, J = 19.5, 6.5 Hz, 6H), 0.92 (t, J = 6.7 Hz, 3H). |
| 29 | 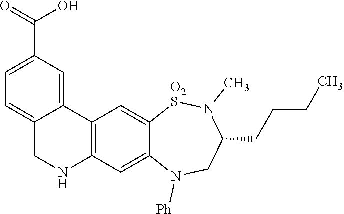 [SMILES: CCCC[C@@H]1CN(c2ccccc2)c2cc3c(cc2S(=O)(=O)N1C)-c1cc(C(=O)O)ccc1CN3] | [M + H] m/z 492.282 | |
| 30 | 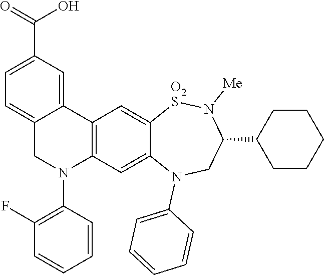 [SMILES: CN1[C@H](C2CCCCC2)CN(c2ccccc2)c2cc3c(cc2S1(=O)=O)-c1cc(C(=O)O)ccc1CN3c1ccccc1F] | [M + H] m/z 612.174 | ¹H NMR (400 MHz, DMSO) δ 13.04 (s, 1H), 7.97 (d, J = 8.2 Hz, 1H), 7.85-7.76 (m, 2H), 7.59 (d, J = 8.1 Hz, 1H), 7.31 (t, J = 7.8 Hz, 2H), 7.09 (s, 1H), 6.99 (dd, J = 14.3, 6.3 Hz, 3H), 6.82 (t, J = 7.7 Hz, 1H), 6.52 (q, J = 7.2 Hz, 1H), 6.25 (t, J = 8.5 Hz, 1H), 6.16 (s, 1H), 4.37 (d, J = 15.5 Hz, 1H), 4.24 (d, J = 5.7 Hz, 2H), 3.77-3.61 (m, 2H), 2.67 (s, 2H), 2.55 (s, 3H), 1.92 (d, J = 16.1 Hz, 1H), 1.72 (s, 2H), 1.61 (s, 2H), 1.17 (d, J = 16.7 Hz, 4H), 1.04-0.93 (m, 2H). |

### Ex. 31: Synthesis of (R)-10-cyclohexyl-6,11-dimethyl-5,6,8,9,10,11-hexahydro-111,2,5]thiadiazepino[7,6-b]phenanthridine-2-carboxylic acid 12,12-dioxide

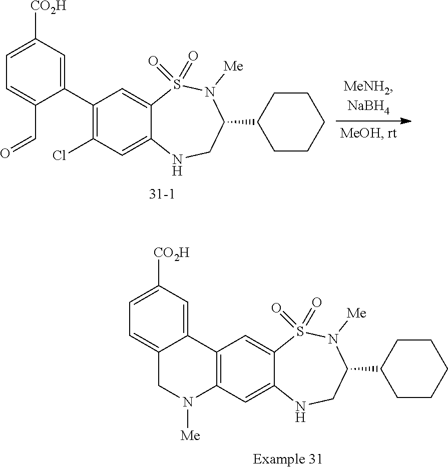 [SMILES: CN1[C@H](C2CCCCC2)CNc2cc(Cl)c(-c3cc(C(=O)O)ccc3C=O)cc2S1(=O)=O ; CN1Cc2ccc(C(=O)O)cc2-c2cc3c(cc21)NC[C@@H](C1CCCCC1)N(C)S3(=O)=O]

### Step 31-1

To a solution of (R)-3-(7-chloro-3-cyclohexyl-2-methyl-1,1-dioxido-2,3,4,5-tetrahydrobenzo[f][1,2,5]thiadiazepin-8-yl)-4-formylbenzoic acid (25 mg, 0.052 mmol) in MeOH (0.52 mL) was added 1 M methanamine in MeOH (157 μL, 0.157 mmol). The reaction was stirred for 2 h, then NaBH₄ (9.9 mg, 0.262 mmol) was added. The reaction mixture was stirred at room temperature for 1 h, then 2N HCl was added and the reaction was extracted with EtOAc. The organic layer was dried over Na₂SO₄, filtered, and concentrated. The residue was then purified by preparative RPHPLC to afford (R)-10-cyclohexyl-6,11-dimethyl-5,6,8,9,10,11-hexahydro-[1,2,5]thiadiazepino[7,6-b]phenanthridine-2-carboxylic acid 12,12-dioxide (3 mg, 6.59 μmol, 12.56% yield) as an off-white solid. ¹H NMR (400 MHz, DMSO) δ 8.08 (d, J=1.6 Hz, 1H), 7.77 (s, 1H), 7.73 (dd, J=7.6, 1.6 Hz, 1H), 7.26 (d, J=8.0 Hz, 1H), 6.71 (s, 1H), 6.14 (s, 1H), 4.34 (dd, J=17.3, 15.3 Hz, 2H), 3.98-3.85 (m, 1H), 3.77 (m, 2H), 2.85 (s, 3H), 2.76 (s, 3H), 2.08-1.97 (m, 1H), 1.71-1.67 (m, 4H), 1.27-1.11 (m, 4H), 1.04-0.90 (m, 2H). ESI MS m/z=456.2 [M+H]⁺.

The following examples were prepared using a procedure similar to that used for Ex. 31:

| Ex. # | Structure | MS |
|---|---|---|
| 32 | 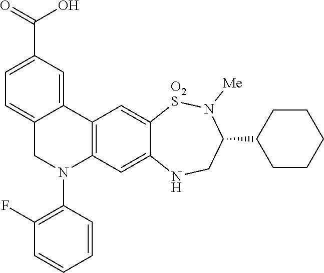 [SMILES: CN1[C@H](C2CCCCC2)CNc2cc3c(cc2S1(=O)=O)-c1cc(C(=O)O)ccc1CN3c1ccccc1F] | [M + H] m/z 536.247 |

### Ex. 33: Synthesis of (R)-10′-butyl-11′-methyl-8′-phenyl-8′,9′,10′,11′-tetrahydro-6′H-spiro[cyclopropane-1,5′-[1,2,5]thiadiazepino[7,6-b]phenanthridine]-2′-carboxylic acid 12′,12′-dioxide

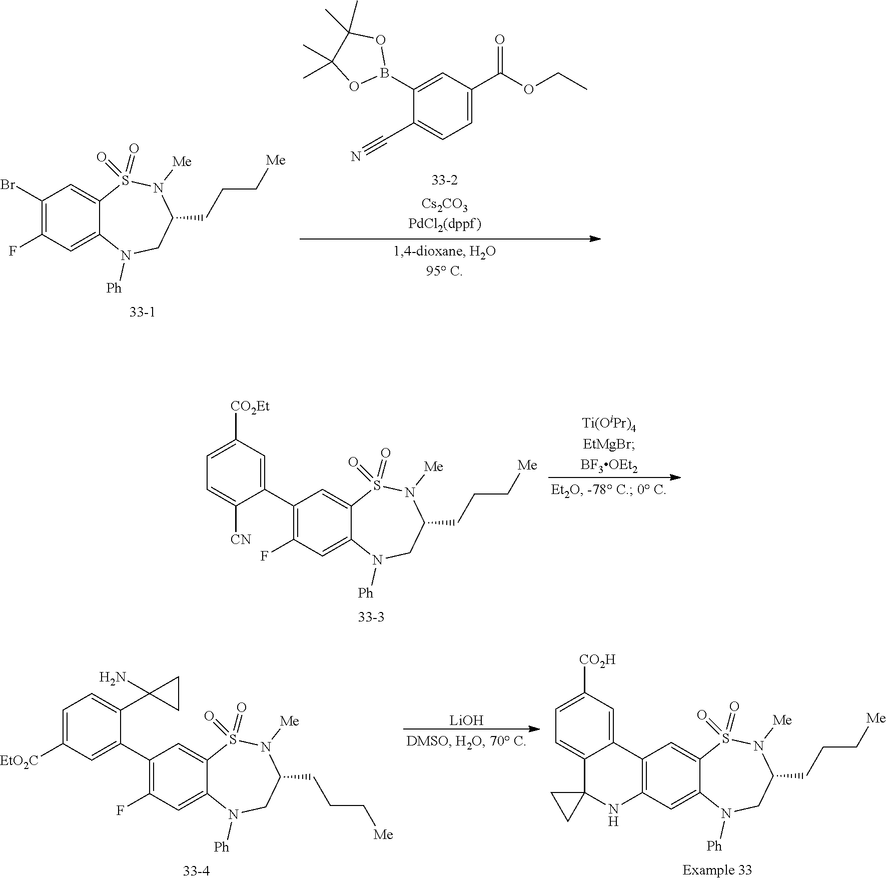 [SMILES: CCCC[C@@H]1CN(c2ccccc2)c2cc(F)c(Br)cc2S(=O)(=O)N1C ; CCOC(=O)c1ccc(C#N)c(B2OC(C)(C)C(C)(C)O2)c1 ; CCCC[C@@H]1CN(c2ccccc2)c2cc(F)c(-c3cc(C(=O)OCC)ccc3C#N)cc2S(=O)(=O)N1C ; CCCC[C@@H]1CN(c2ccccc2)c2cc(F)c(-c3cc(C(=O)OCC)ccc3C3(N)CC3)cc2S(=O)(=O)N1C ; "LiOH" ; CCCC[C@@H]1CN(c2ccccc2)c2cc3c(cc2S(=O)(=O)N1C)-c1cc(C(=O)O)ccc1C1(CC1)N3]

### Step 33-1

To a vial containing (R)-8-bromo-3-butyl-7-fluoro-2-methyl-5-phenyl-2,3,4,5-tetrahydrobenzo[f][1,2,5]thiadiazepine 1,1-dioxide (200 mg, 0.453 mmol), ethyl 4-cyano-3-(4,4,5,5-tetramethyl-1,3,2-dioxaborolan-2-yl)benzoate (273 mg, 0.906 mmol), cesium carbonate (443 mg, 1.359 mmol), and PdCl₂(dppf) (33.2 mg, 0.045 mmol) was added dioxane (3.94 mL) and H₂O (0.591 mL) under N₂. The reaction was stirred and heated at 95° C. overnight. The reaction was cooled to room temperature and then quenched with HCl and extracted with EtOAc. The organic layer was dried, concentrated, then purified by column silica gel column chromatography (0-25% ethyl acetate/cyclohexane) to provide ethyl (R)-3-(3-butyl-7-fluoro-2-methyl-1,1-dioxido-5-phenyl-2,3,4,5-tetrahydrobenzo[f][1,2,5]thiadiazepin-8-yl)-4-cyanobenzoate (43.2 mg, 0.081 mmol, 17.80% yield). ESI MS m/z=536.4 [M+H]⁺.

### Step 33-2

In a vial equipped with a stir bar, ethyl (R)-3-(3-butyl-7-fluoro-2-methyl-1,1-dioxido-5-phenyl-2,3,4,5-tetrahydrobenzo[f][1,2,5]thiadiazepin-8-yl)-4-cyanobenzoate (43 mg, 0.080 mmol) was dissolved in Et₂O (0.535 ml) under N₂. The solution was cooled to −78° C., and titanium(IV) isopropoxide (28.2 μL, 0.096 mmol) was added in one portion. Then, ethylmagnesium bromide (64.2 μL, 0.193 mmol) was added dropwise over the course of 10 minutes at −78° C. The reaction mixture was stirred for 45 minutes and then warmed to 0° C. and stirred for 90 minutes. To the reaction mixture was added BF3·OEt2 (20.35 μL, 0.161 mmol) dropwise over the course of 2 minutes and the reaction mixture warmed to room temperature overnight. The mixture was quenched with H₂O and was then extracted with EtOAc, dried over Na₂SO₄, and concentrated to provide ethyl (R)-4-(1-aminocyclopropyl)-3-(3-butyl-7-fluoro-2-methyl-1,1-dioxido-5-phenyl-2,3,4,5-tetrahydrobenzo[f][1,2,5]thiadiazepin-8-yl)benzoate (5 mg, 8.84 μmol, 11.01% yield) which was used without further purification in the next step. ESI MS m/z=566.4 [M+H]⁺.

### Step 33-3

The crude ethyl (R)-4-(1-aminocyclopropyl)-3-(3-butyl-7-fluoro-2-methyl-1,1-dioxido-5-phenyl-2,3,4,5-tetrahydrobenzo[f][1,2,5]thiadiazepin-8-yl)benzoate (5 mg, 8.84 μmol) was redissolved in DMSO (0.5 mL) and H₂O (0.5 mL), then LiOH was added and the reaction was heated to 70° C. Upon completion (monitored by LC-MS), the reaction was quenched with formic acid (50 L) and was directly subjected to preparative RPHPLC to provide the cyclized and saponified product (R)-10′-butyl-11′-methyl-8′-phenyl-8′,9′,10′,11′-tetrahydro-6′H-spiro[cyclopropane-1,5′-[1,2,5]thiadiazepino[7,6-b]phenanthridine]-2′-carboxylic acid 12′,12′-dioxide (2 mg, 3.86 μmol, 4.81% yield). ¹HNMR (400 MHz, DMSO) δ 9.18 (s, 1H), 9.11 (s, 1H), 8.44 (d, J=8.7 Hz, 1H), 8.30 (dd, J=8.4, 1.5 Hz, 1H), 7.83 (s, 1H), 7.22 (t, J=7.9 Hz, 2H), 6.82 (d, J=7.4 Hz, 3H), 6.66 (s, 1H), 4.17 (d, J=15.8 Hz, 1H), 4.00-3.88 (m, 1H), 3.46-3.37 (m, 1H), 2.58 (s, 3H), 1.68-1.52 (m, 2H), 1.47-1.31 (m, 6H), 1.23 (s, 1H), 1.15 (s, 1H), 0.93 (t, J=6.9 Hz, 3H). ESI MS m/z=518.4 [M+H]⁺.

### Ex. 34: Synthesis of (R)-3-butyl-2-methyl-5-phenyl-2,3,4,5-tetrahydrobenzo[2,3]benzofuro[6,5-f][1,2,5]thiadiazepine-10-carboxylic acid 1,1-dioxide

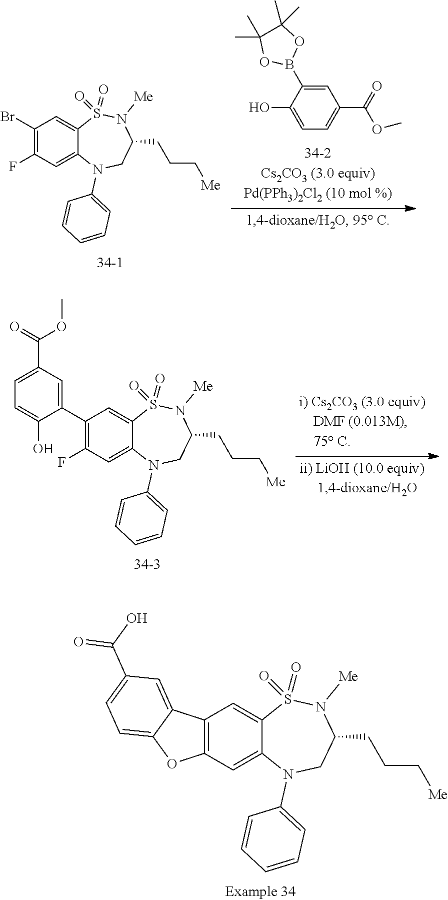

### Step 34-1

In a 1 dram vial equipped with a stir bar, (R)-8-bromo-3-butyl-7-fluoro-2-methyl-5-phenyl-2,3,4,5-tetrahydrobenzo[f][1,2,5]thiadiazepine 1,1-dioxide (30.0 mg, 0.07 mmol, 1.0 equiv), methyl 4-hydroxy-3-(4,4,5,5-tetramethyl-1,3,2-dioxaborolan-2-yl)benzoate (28.4 mg, 0.10 mmol, 1.5 equiv), cesium carbonate (66.4 mg, 0.20 mmol, 3.0 equiv), and bis(triphenylphosphine)palladium(II) dichloride (4.8 mg, 0.007 mmol, 10 mol %) were combined neat under an atmosphere of nitrogen. Next, 1,4-dioxane (0.68 mL) and water (0.1 mL) were added. The vial was sealed with electrical tape, and the reaction was heated at 95° C. for 3 h. Upon cooling to room temperature, the mixture was concentrated and purified by RPHPLC to afford methyl (R)-3-(3-butyl-7-fluoro-2-methyl-1,1-dioxido-5-phenyl-2,3,4,5-tetrahydrobenzo[f][1,2,5]thiadiazepin-8-yl)-4-hydroxybenzoate (23.6 mg, 68% yield). ESI MS m/z=513.2 [M+H]⁺.

### Step 34-2

In a 20 mL vial equipped with a stir bar, methyl (R)-3-(3-butyl-7-fluoro-2-methyl-1,1-dioxido-5-phenyl-2,3,4,5-tetrahydrobenzo[f][1,2,5]thiadiazepin-8-yl)-4-hydroxybenzoate (23.6 mg, 0.05 mmol, 1.0 equiv) was dissolved in N,N-dimethylformamide (1.0 mL, 0.013M). Next, cesium carbonate (45.0 mg, 0.14 mmol, 3.0 equiv) was added, and the mixture was heated at 75° C. for 30 minutes. Upon cooling to room temperature, the solvent was removed using a Biotage® V-10 evaporator. The crude residue was re-dissolved in 1,4-dioxane (2.0 mL) and water (0.50 mL). Lithium hydroxide (24.0 mg, 10.0 equiv) was then added, and the resulting mixture was stirred at room temperature for 15 h. The reaction mixture was treated with formic acid (0.50 mL) and concentrated. The residue was purified by RPHPLC to afford (R)-3-butyl-2-methyl-5-phenyl-2,3,4,5-tetrahydrobenzo[2,3]benzofuro[6,5-f][1,2,5]thiadiazepine-10-carboxylic acid 1,1-dioxide (13.5 mg, 61% yield). ESI MS m/z=479.2 [M+H]⁺.

### Ex. 35: Synthesis of (R)-12-butyl-13-methyl-10-phenyl-6,7,10,11,12,13-hexahydrobenzo[7′,8′][1,4]dioxocino[6′,5′:4,5]benzo[1,2-f][1,2,5]thiadiazepine-2-carboxylic acid 14,14-dioxide

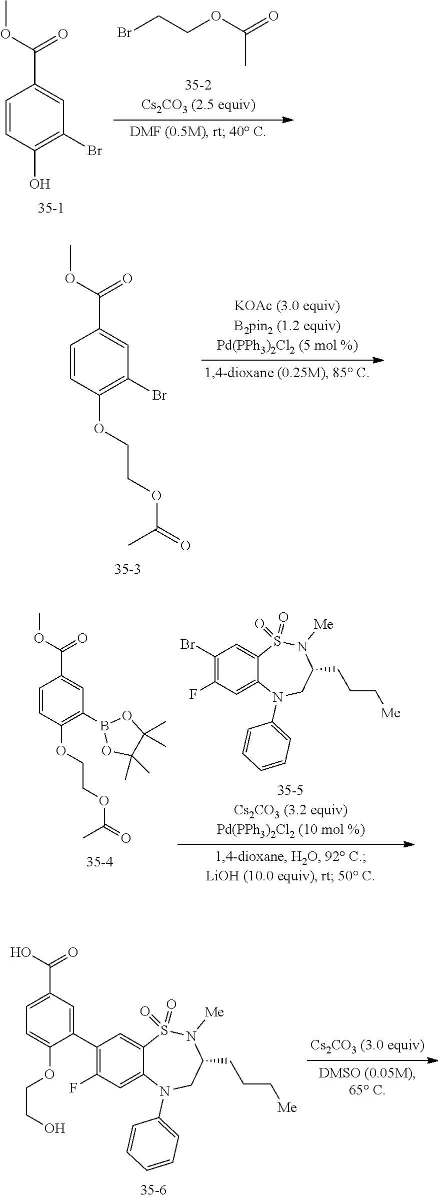

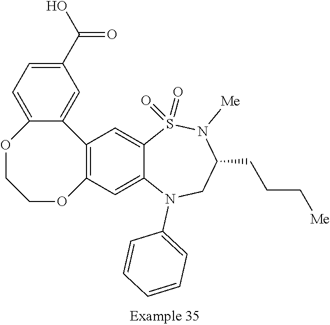 [SMILES: CCCC[C@@H]1CN(c2ccccc2)c2cc3c(cc2S(=O)(=O)N1C)-c1cc(C(=O)O)ccc1OCCO3]

### Step 35-1

In a 40 mL vial equipped with a stir bar, methyl 3-bromo-4-hydroxybenzoate (1.00 g, 4.3 mmol, 1.0 equiv) was dissolved in N,N-dimethylformamide (8.7 mL, 0.5M). Next, cesium carbonate (3.53 g, 10.8 mmol, 2.5 equiv) was added, followed by 2-bromoethyl acetate (720 mL, 6.5 mmol, 1.5 equiv). The resulting mixture was stirred at room temperature for 1 h. Additional 2-bromoethyl acetate (720 mL, 6.5 mmol, 1.5 equiv) was added, and the mixture was then heated at 40° C. for 16 h. The reaction mixture was filtered through silica gel using ethyl acetate to rinse and concentrated. The crude residue was purified by silica gel column chromatography to afford methyl 4-(2-acetoxyethoxy)-3-bromobenzoate (1.33 g, 97% yield). ESI MS m/z=319.2 [M+H]⁺.

### Step 35-2

In a 40 mL vial equipped with a stir bar, methyl 4-(2-acetoxyethoxy)-3-bromobenzoate (658.0 mg, 2.1 mmol, 1.0 equiv), potassium acetate (610.9 mg, 6.2 mmol, 3.0 equiv), bis(pinacolato)diboron (632.3 mg, 2.5 mmol, 1.2 equiv), and bis(triphenylphosphine)palladium(II) dichloride (72.8 mg, 0.10 mmol, 5 mol %) were combined neat under a nitrogen atmosphere. Next, 1,4-dioxane (8.2 mL, 0.25M) was added, and the mixture was heated at 85° C. for 19 h. Upon cooling to room temperature, the mixture was concentrated and purified by RPHPLC to afford methyl 4-(2-acetoxyethoxy)-3-(4,4,5,5-tetramethyl-1,3,2-dioxaborolan-2-yl)benzoate (169.6 mg, 22% yield). ESI MS m/z=365.2 [M+H]⁺.

### Step 35-3

In a 20 mL vial equipped with a stir bar, methyl 4-(2-acetoxyethoxy)-3-(4,4,5,5-tetramethyl-1,3,2-dioxaborolan-2-yl)benzoate (169.6 mg, 0.47 mmol, 1.5 equiv), (R)-8-bromo-3-butyl-7-fluoro-2-methyl-5-phenyl-2,3,4,5-tetrahydrobenzo[f][1,2,5]thiadiazepine 1,1-dioxide (137.0 mg, 0.31 mmol, 1.0 equiv), cesium carbonate (328.7 mg, 1.0 mmol, 3.2 equiv), and bis(triphenylphosphine)palladium(II) dichloride (21.8 mg, 0.03 mmol, 10 mol %) were combined neat under a nitrogen atmosphere. Next, 1,4-dioxane (3.1 mL) and water (0.47 mL) were added, and the mixture was heated at 92° C. for 1 h. Upon cooling to room temperature, lithium hydroxide (74.3 mg, 3.1 mmol, 10.0 equiv) was added and the mixture was stirred for 5 h at room temperature. After this time, additional lithium hydroxide (74.3 mg, 3.1 mmol, 10.0 equiv) was added, and the mixture was stirred for 15 h at room temperature. The mixture was then heated at 50° C. for 6 h. The reaction mixture was then concentrated, and the residue was purified by RPHPLC to afford (R)-3-(3-butyl-7-fluoro-2-methyl-1,1-dioxido-5-phenyl-2,3,4,5-tetrahydrobenzo[f][1,2,5]thiadiazepin-8-yl)-4-(2-hydroxyethoxy)benzoic acid (50.0 mg, 30% yield). ESI MS m/z=543.2 [M+H]⁺.

### Step 35-4

In a 20 mL vial equipped with a stir bar, (R)-3-(3-butyl-7-fluoro-2-methyl-1,1-dioxido-5-phenyl-2,3,4,5-tetrahydrobenzo[f][1,2,5]thiadiazepin-8-yl)-4-(2-hydroxyethoxy)benzoic acid (50.0 mg) was dissolved in dimethyl sulfoxide (1.8 mL, 0.05M). Cesium carbonate (90.0 mg, 0.27 mmol, 3.0 equiv) was then added, and the reaction mixture was heated at 65° C. overnight. Upon cooling to room temperature, formic acid (0.5 mL) was added, and the mixture was passed through a 0.45 micron syringe filter. Purification by RPHPLC afforded (R)-12-butyl-13-methyl-10-phenyl-6,7,10,11,12,13-hexahydrobenzo[7′,8′][1,4]dioxocino[6′,5′:4,5]benzo[11,2-f] [1,2,5]thiadiazepine-2-carboxylic acid 14,14-dioxide (18.2 mg, 38% yield). ESI MS m/z=523.2 [M+H]⁺.

### Ex. 36: Synthesis of (R)-9-cyclobutyl-10-methyl-7-phenyl-7,8,9,10-tetrahydro-4H-thieno[3′,2′:3,4]chromeno[7,6-f][1,2,5]thiadiazepine-2-carboxylic acid 11,11-dioxide

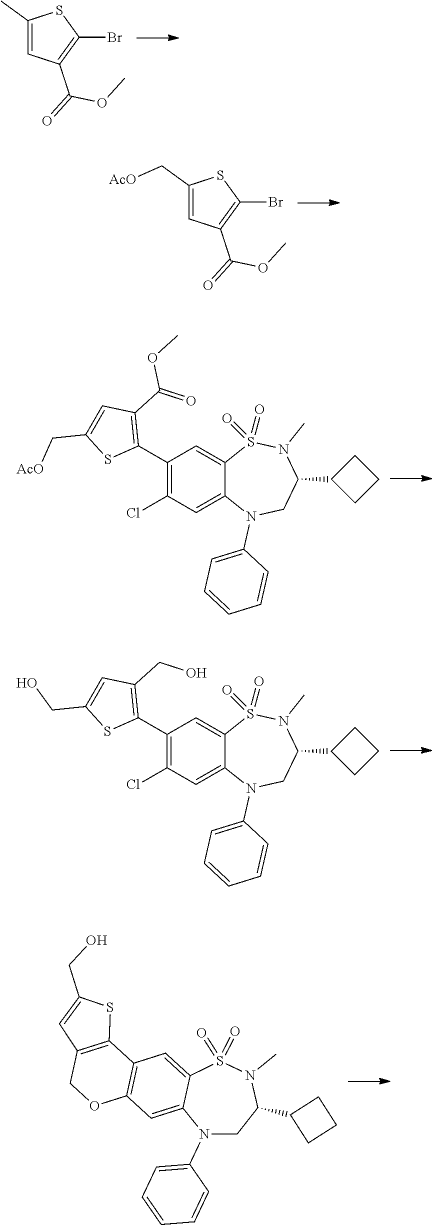

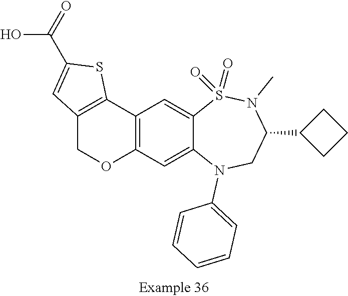 [SMILES: CN1[C@H](C2CCC2)CN(c2ccccc2)c2cc3c(cc2S1(=O)=O)-c1sc(C(=O)O)cc1CO3]

### Step 36-1

A solution of methyl 2-bromo-5-methylthiophene-3-carboxylate (701 mg, 2.98 mmol) and carbon tetrachloride (11.9 mL) was stirred vigorously under nitrogen for 15 minutes at ambient temperature. After 15 minutes, AIBN (14.7 mg, 0.089 mmol) and NBS (584 mg, 3.28 mmol) were quickly added in one portion. The suspension was then slowly warmed to 80° C. under positive nitrogen pressure. After 3 h, the mixture was cooled to ambient temperature and filtered. The resulting yellow solution was concentrated under a nitrogen stream and the residue obtained therefrom was redissolved in acetonitrile (12 mL). Potassium acetate (1.75 g, 17.9 mmol) was then added in one portion and the mixture was heated to 70° C. for 90 min. The mixture was diluted with MTBE and filtered through celite, rinsing with MTBE. The filtrate was concentrated and the residue was subjected to silica gel column chromatography (0-40% ethyl acetate in cyclohexane) to afford methyl 5-(acetoxymethyl)-2-bromothiophene-3-carboxylate (503 mg, 1.72 mmol, 58% yield). ¹H NMR (400 MHz, CDCl3) δ 7.34 (d, J=0.8 Hz, 1H), 5.12 (d, J=0.9 Hz, 2H), 3.87 (s, 3H), 2.09 (s, 3H). [M-AcOH+H] m/z 232.927.

### Step 36-2

A mixture of (R)-7-chloro-3-cyclobutyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (220 mg, 0.44 mmol), PdCl₂(PPh₃)₂ (61 mg, 0.087 mmol), methyl 5-(acetoxymethyl)-2-bromothiophene-3-carboxylate (190 mg, 0.65 mmol), 2 M aqueous cesium carbonate solution (1.1 mL, 2.2 mmol), and THF (4.4 mL) was heated to 70° C. for 40 min. The mixture was then cooled to ambient temperature, the aqueous layer was removed by pipet, and the organic layer was concentrated directly. The residue was subjected to silica gel column chromatography (0-40% ethyl acetate in cyclohexane), to afford semi-purified methyl (R)-5-(acetoxymethyl)-2-(7-chloro-3-cyclobutyl-2-methyl-1,1-dioxido-5-phenyl-2,3,4,5-tetrahydrobenzo[f][1,2,5]thiadiazepin-8-yl)thiophene-3-carboxylate (assumed 0.44 mmol, 100% yield). 1H NMR (400 MHz, CDCl3) δ 7.93 (s, 1H), 7.60 (dddd, J=7.0, 5.5, 4.4, 1.3 Hz, 3H), 7.50 (s, 1H), 7.07-7.00 (m, 1H), 7.00-6.95 (m, 2H), 5.23 (s, 2H), 3.99 (d, J=15.7 Hz, 1H), 3.78 (t, J=10.5 Hz, 1H), 3.73 (s, 3H), 3.47 (t, J=13.2 Hz, 1H), 2.71 (s, 3H), 2.51 (q, J=8.5 Hz, 1H), 2.03-1.81 (m, 4H), 1.77 (q, J=8.2 Hz, 1H), 1.55 (s, 1H). [M+H] m/z 589.000.

### Step 36-3

DIBAL-H (3.5 mL, 1 M in toluene, 3.5 mmol) was added dropwise at −78° C. to a stirred solution of methyl (R)-5-(acetoxymethyl)-2-(7-chloro-3-cyclobutyl-2-methyl-1,1-dioxido-5-phenyl-2,3,4,5-tetrahydrobenzo[f][1,2,5]thiadiazepin-8-yl)thiophene-3-carboxylate (assumed 0.44 mmol) and DCM (4.4 mL). After 40 min at −78° C., MeOH (1 mL) was added carefully and the mixture was warmed to rt. Saturated aqueous Rochelle's salt solution (4 mL) was then added and the mixture was stirred vigorously for 30 min. Significant precipitated solids were filtered off and the filtrate was partitioned between MTBE and brine. The aqueous phase was extracted with MTBE and the combined orgnaic layers were dried over magnesium sulfate, filtered, and concentrated. The residue was subjected to silica gel column chromatography (0-100% ethyl acetate in cyclohexane) to afford (R)-8-(3,5-bis(hydroxymethyl)thiophen-2-yl)-7-chloro-3-cyclobutyl-2-methyl-5-phenyl-2,3,4,5-tetrahydrobenzo[f][1,2,5]thiadiazepine 1,1-dioxide (30 mg, 0.058 mmol, 13% over 2 steps). 1H NMR (400 MHz, CDCl3) δ 7.94 (s, 1H), 7.38-7.28 (m, 2H), 7.14 (d, J=19.7 Hz, 2H), 7.08-6.93 (m, 4H), 4.85-4.79 (m, 2H), 4.45 (s, 2H), 4.00 (d, J=15.1 Hz, 1H), 3.75 (t, J=10.7 Hz, 1H), 3.49 (t, J=13.4 Hz, 1H), 2.70 (s, 3H), 2.53 (d, J=8.7 Hz, 1H), 2.11 (m, 2H), 2.00-1.75 (m, 4H). [M+H-H₂O] m/z 501.000.

### Step 36-4

(R)-8-(3,5-bis(hydroxymethyl)thiophen-2-yl)-7-chloro-3-cyclobutyl-2-methyl-5-phenyl-2,3,4,5-tetrahydrobenzo[f][1,2,5]thiadiazepine 1,1-dioxide (30 mg, 0.058 mmol) was dissolved in DMSO (0.75 mL) and cesium carbonate (113 mg, 0.35 mmol) was added. The mixture was then heated to 100° C. for 1 h before cooling to ambient temperature. The reaction mixture was partitioned between MTBE and water, then the organic layer was removed. The aqueous layer was treated with 1 M tartaric acid solution and extracted once more with MTBE. The combined organic layers were washed with brine, dried over magnesium sulfate, filtered and concentrated. The resulting crude (R)-9-cyclobutyl-2-(hydroxymethyl)-10-methyl-7-phenyl-7,8,9,10-tetrahydro-4H-thieno[3′,2′:3,4]chromeno[7,6-f][1,2,5]thiadiazepine 11,11-dioxide (assumed 0.062 mmol, 100% yield) was taken forward without further purification. [M+H] m/z 483.000.

### Step 36-5

Sulfur trioxide pyridine complex (49 mg, 0.31 mmol) was added at 0° C. to a stirred solution of crude (R)-9-cyclobutyl-2-(hydroxymethyl)-10-methyl-7-phenyl-7,8,9,10-tetrahydro-4H-thieno[3′,2′:3,4]chromeno[7,6-f][1,2,5]thiadiazepine 11,11-dioxide (assumed 0.062 mmol) in iPr₂NEt (43 μL, 0.25 mmol), DMSO (35 μL, 0.50 mmol), and DCM (1.2 mL). After 10 min, the reaction mixture was partitioned between MTBE and 1 N HCl. The organic phase was separated and washed with brine, dried over magnesium sulfate, filtered, and concentrated. The residue obtained therefrom (assumed 0.062 mmol) was then redissolved in tBuOH (0.62 mL) and water (0.62 mL). Anhydrous sodium monophosphate (30 mg, 0.25 mmol), 2-methyl-2-butene (2 M in THF, 250 μL, 0.50 mmol), and sodium chlorite (80% by weight, 14 mg, 0.125 mmol) were then sequentially added. After 30 min, 9 mg additional sodium chlorite was added. After 15 minutes further, the reaction mixture was filtered and purified directly by HPLC to afford (R)-9-cyclobutyl-10-methyl-7-phenyl-7,8,9,10-tetrahydro-4H-thieno[3′,2′:3,4]chromeno[7,6-f][1,2,5]thiadiazepine-2-carboxylic acid 11,11-dioxide (7 mg, 0.014 mmol, 23% overall yield). 1H NMR (400 MHz, CDCl3) δ 7.98 (s, 1H), 7.59 (s, 1H), 7.33-7.29 (m, 2H), 7.00 (tt, J=7.4, 1.1 Hz, 1H), 6.97-6.92 (m, 2H), 6.66 (s, 1H), 5.38-5.24 (m, 2H), 3.98 (d, J=15.6 Hz, 1H), 3.81 (t, J=10.5 Hz, 1H), 3.44 (d, J=14.0 Hz, 1H), 2.71 (s, 3H), 2.15 (dd, J=7.7, 3.4 Hz, 2H), 2.02-1.73 (m, 5H). [M+H] m/z 496.986.

Starting from the appropriate primary amine, the following compounds were prepared using a procedure analogous to that used for Ex.4 and Ex.12:

| Ex. | | |
|---|---|---|
| # | Structure | MS |
| 37 | 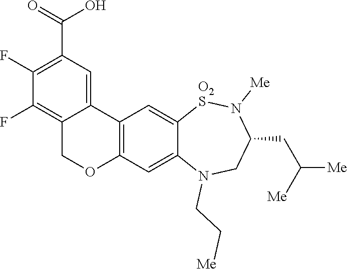 [SMILES: CCCN1C[C@@H](CC(C)C)N(C)S(=O)(=O)c2cc3c(cc21)OCc1c-3cc(C(=O)O)c(F)c1F] | [M + H] m/z 495.2 |
| 38 | 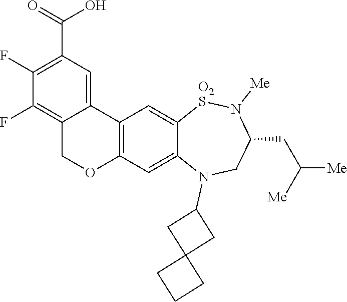 [SMILES: CC(C)C[C@@H]1CN(C2CC3(CCC3)C2)c2cc3c(cc2S(=O)(=O)N1C)-c1cc(C(=O)O)c(F)c(F)c1CO3] | [M + H] m/z 547.2 |
| 39 | 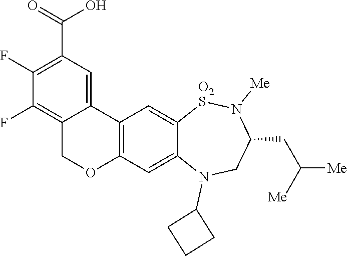 [SMILES: CC(C)C[C@@H]1CN(C2CCC2)c2cc3c(cc2S(=O)(=O)N1C)-c1cc(C(=O)O)c(F)c(F)c1CO3] | [M + H] m/z 507.2 |
| 40 | 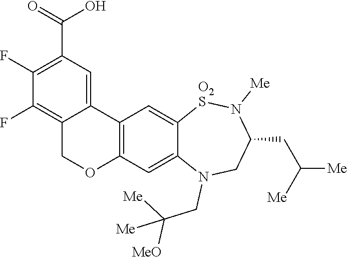 [SMILES: COC(C)(C)CN1C[C@@H](CC(C)C)N(C)S(=O)(=O)c2cc3c(cc21)OCc1c-3cc(C(=O)O)c(F)c1F] | [M + H] m/z 539.2 |

### Biological Activity

### Methods:

HepG2-NTCP A3 cells were maintained in DMEM media supplemented with GlutaMAX™, 10% fetal bovine serum, 1% penicillin/streptomycin, and 5 ug/mL puromycin at 37° C. in a humidified atmosphere with 5% CO₂ in a collagen-coated tissue culture flask.

HepG2-NTCP cells were seeded in 384 well plate containing 16,000 cells/well two days prior to the infection. On the day of infection, compounds were 3-fold serially diluted in DMSO and pre-incubated with HepG2-NTCP cells for two hours before purified HBV addition. HBV infection was carried out at 2000 GE/cell with 4% PEG, and the final concentration of DMSO is 0.5%. On day one post infection, HBV-containing media was aspirated, cells were washed once with PBS and then maintained in 2.5% DMSO containing media for the remainder of the assay.

Supernatants from infected HepG2-NTCP cells were collected at day 8 post infection, and the amount of viral antigen HBeAg was measured by HBeAg AlphaLISA detection kit (PerkinElmer) following the manufacturer's recommended protocol. Irrespective of readout, compound concentrations that reduce viral product accumulation in supernatants by 50% relative to DMSO controls (EC₅₀) are reported. EC₅₀ ranges are as follows: A<0.1 □M; B 0.1-1 □M; C>1□M.

TABLE 1
Summary of HBV Activities

| Example | Cellular | Example | Cellular |
|---|---|---|---|
| Number | EC₅₀ (□M) | Number | EC₅₀ (□M) |
| 1 | B | 2 | B |
| 3 | B | 4 | A |
| 5 | A | 6 | B |
| 7 | A | 8 | A |
| 9 | C | 10 | A |
| 11 | C | 12 | A |
| 13 | A | 14 | A |
| 15 | A | 16 | A |
| 17 | A | 18 | A |
| 19 | A | 20 | — |
| 21a | B | 21b | B |
| 22a | C | 22b | C |
| 23 | B | 24 | B |
| 25 | C | 26 | — |
| 27 | A | 28 | B |
| 29 | B | 30 | C |
| 31 | C | 32 | B |
| 33 | C | 34 | B |
| 35 | B | 36 | A |
| 37 | A | 38 | A |
| 39 | B | 40 | B |

HDV virus collected from the supernatants of HuH7-END cells was purified in the presence of 6% polyethylene glycol (PEG). Viral titer was then quantified by RT-qPCR using HDV specific primers against a reference standard. For HDV infection, HepG2-NTCP cells were seeded in 96-well plates at 60,000 cells/well. On the day of infection, compounds were 4-fold serially diluted in DMSO and pre-incubated with HepG2-NTCP cells for two hours before infecting with 800 GE/cell of HDV in 4% PEG. On day one post infection, HDV-containing media was aspirated, cells were washed once with PBS and then maintained in 2.5% DMSO containing media for the remainder of the assay. Viral RNA was isolated using RNeasy kits (Qiagen) at day 12 post infection and reverse transcribed into cDNA using High-Capacity RNA-to-cDNA Kit (Thermo Fisher). Relative HDV gene expression was quantified by RT-qPCR after normalization to expression of a housekeeping gene. Irrespective of readout, compound concentrations that reduce viral RNA by 50% relative to DMSO controls (EC₅₀) are reported. EC₅₀ ranges are as follows: A<0.1 □M; B 0.1-1 □M; C>1 □M.

TABLE 2
Summary of HDV Activities

| Example | Cellular | Example | Cellular |
|---|---|---|---|
| Number | EC₅₀ (□M) | Number | EC₅₀ (□M) |
| 1 | A | — | — |

While this invention has been particularly shown and described with references to preferred embodiments thereof, it will be understood by those skilled in the art that various changes in form and details may be made therein without departing from the scope of the invention encompassed by the appended claims.

1. A compound represented by Formula (I): 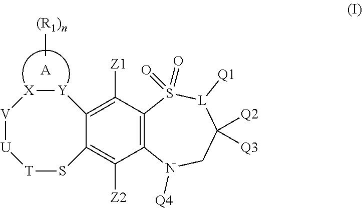
or a pharmaceutically acceptable salt thereof, wherein:
L is CR₁₄ or N;
R₁₄ is hydrogen, optionally substituted —C₁-C₆ alkyl, optionally substituted —C₂-C₆ alkenyl, optionally substituted —C₂-C₆ alkynyl, or optionally substituted —C₁-C₆ alkoxy;
when L is N, Q1 is selected from the group consisting of hydrogen, optionally substituted —C₁-C₆ alkyl, optionally substituted —C₂-C₆ alkenyl, optionally substituted —C₃-C₈ cycloalkyl, optionally substituted —C₃-C₈ cycloalkenyl, optionally substituted 3- to 8-membered heterocycloalkyl, optionally substituted aryl, optionally substituted heteroaryl, optionally substituted arylalkyl, and optionally substituted heteroarylalkyl;
when L is CR₁₄, Q1 is selected from the group consisting of hydrogen, optionally substituted —C₁-C₆ alkyl, optionally substituted —C₂-C₆ alkenyl, optionally substituted the group consisting of —C₁-C₆ alkoxy, optionally substituted —C₃-C₈ cycloalkyl, optionally substituted —C₃-C₈ cycloalkenyl, optionally substituted 3- to 8-membered heterocycloalkyl, optionally substituted aryl, optionally substituted heteroaryl; optionally substituted arylalkyl, and optionally substituted heteroarylalkyl;
Q2 and Q3 are each independently selected from the group consisting of hydrogen, optionally substituted —C₁-C₆ alkyl, optionally substituted —C₂-C₆ alkenyl, optionally substituted the group consisting of —C₁-C₆ alkoxy, optionally substituted —C₃-C₈ cycloalkyl, optionally substituted —C₃-C₈ cycloalkenyl, optionally substituted 3- to 8-membered heterocycloalkyl, optionally substituted aryl, optionally substituted heteroaryl; optionally substituted arylalkyl, and optionally substituted heteroarylalkyl;
Q4 is selected from the group consisting of hydrogen, optionally substituted —C₁-C₆ alkyl, optionally substituted —C₂-C₆ alkenyl, optionally substituted —C₃-C₈ cycloalkyl, optionally substituted —C₃-C₈ cycloalkenyl, optionally substituted 3- to 8-membered heterocycloalkyl, optionally substituted aryl, optionally substituted heteroaryl; optionally substituted arylalkyl, and optionally substituted heteroarylalkyl;
Z1 and Z2 are each independently selected from the group consisting of:
1) hydrogen;
2) halogen;
3) —NO₂;
4) Cyano;
5) Optionally substituted —C₁-C₈ alkyl;
6) Optionally substituted —C₂-C₈ alkenyl;
7) Optionally substituted —C₂-C₈ alkynyl;
8) Optionally substituted —C₃-C₈ cycloalkyl;
9) Optionally substituted 3- to 12-membered heterocycloalkyl;
10) Optionally substituted aryl;
11) Optionally substituted arylalkyl;
12) Optionally substituted heteroaryl;
13) Optionally substituted heteroarylalkyl;
14) —SR₁₁;
15) —S(O)₂R₁₁;
16) —S(O)₂N(R₁₁)(R₁₂);
17) —C(O)R₁₁;
18) —C(O)OR₁₁;
19) —C(O)N(R₁₁)(R₁₂);
20) —C(O)N(R₁₁)S(O)₂(R₁₂);
21) —N(R₁₁)(R₁₂);
22) —N(R₁₃)C(O)N(R₁₁)(R₁₂);
23) —N(R₁₁)C(O)(R₁₂);
24) —N(R₁₁)C(O)₂(R₁₂);
25) —N(R₁₃)S(O)₂N(R₁₁)(R₁₂);
26) —N(R₁₁)S(O)₂(R₁₂);
27) —OR₁₁;
28) —OC(O)R₁₁;
29) —OC(O)OR₁₁; and
30) —OC(O)N(R₁₁)(R₁₂);
R₁₁, R₁₂, and R₁₃ are each independently selected from the group consisting of hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, and optionally substituted heteroaryl;
alternatively, R₁ and R₁₂ are taken together with the nitrogen atom to which they attached to form an optionally substituted 3-8 membered heterocyclic containing 0, 1, 2, or 3 double bonds;
ring A is a 6- to 12-membered aromatic ring or a 5- to 12-membered heteroaromatic ring containing 0-3 heteroatoms independently selected from N, O, and S wherein X and Y are the atoms at the site of ring fusion;
X is C or N;
Y is C or N;
two of S, T, U and V are selected from the group consisting of O, C(R₁₅)(R₁₆), N(R₁₇), S, S(O), S(O)₂, C(O); and the remainder of S, T, U and V are independently C(R₁₅)(R₁₆); alternatively one, two, or all of U, V, and T are absent;
R₁₅ and R₁₆, are each independently selected from the group consisting of hydrogen, optionally substituted —C₁-C₈ alkyl, halogen, hydroxy, optionally substituted —C₁-C₆ alkoxy, optionally substituted —C₂-C₈ alkenyl, optionally substituted —C₃-C₈ cycloalkyl, optionally substituted 3- to 8-membered heterocycloalkyl, optionally substituted aryl, and optionally substituted heteroaryl;
R₁₇ is selected from hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, and optionally substituted heteroaryl;
alternatively R₁₅ and R₁₆, are taken together with the carbon atom to which they are attached to form an optionally substituted 3-8 membered heterocyclic or carbocyclic ring containing 0, 1, 2, or 3 double bonds;
n is 0, 1, 2, 3, or 4;
each R₁ is independently selected from the group consisting of:
1) halogen;
2) —NO₂;
3) Cyano;
4) Optionally substituted —C₁-C₈ alkyl;
5) Optionally substituted —C₂-C₈ alkenyl;
6) Optionally substituted —C₂-C₈ alkynyl;
7) Optionally substituted —C₃-C₈ cycloalkyl;
8) Optionally substituted 3- to 12-membered heterocycloalkyl;
9) Optionally substituted aryl;
10) Optionally substituted arylalkyl;
11) Optionally substituted heteroaryl;
12) Optionally substituted heteroarylalkyl;
13) —SR₂₇;
14) —SO₃H;
15) —S(O)₂R₂₇;
16) —S(O)₂N(R₂₇)(R₂₈);
17) —PO₃H₂;
18) —C(O)R₂₇;
19) —C(O)OR₂₇;
20) —C(O)N(R₂₇)(R₂₈);
21) —C(O)N(R₂₇)S(O)₂(R₂₈);
22) —N(R₂₇)(R₂₈);
23) —N(R₂₉)C(O)N(R₂₇)(R₂₈);
24) —N(R₂₇)C(O)(R₂₈);
25) —N(R₂₇)C(O)₂(R₂₈);
26) —N(R₂₉)S(O)₂N(R₂₇)(R₂₈);
27) —N(R₂₇)S(O)₂(R₂₈);
28) —OR₂₇;
29) —OC(O)R₂₇;
30) —OC(O)OR₂₇; and
31) —OC(O)N(R₂₇)(R₂₈);
wherein R₂₇, R₂₈, R₂₉, are each independently selected from the group consisting of hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, and optionally substituted heteroaryl;
alternatively, R₂₇ and R₂₈ are taken together with the nitrogen atom to which they attached to form an optionally substituted 3-8 membered heterocyclic containing 0, 1, 2, or 3 double bonds; alternatively, R₂₇ and R₂₈ are taken together with the nitrogen atom to which they attached to form an optionally substituted 3-8 membered heterocyclic containing 0, 1, 2, or 3 double bonds.

2. The compound of claim 1, represented by Formula (V-1) or Formula (V-2), or a pharmaceutically acceptable salt thereof: 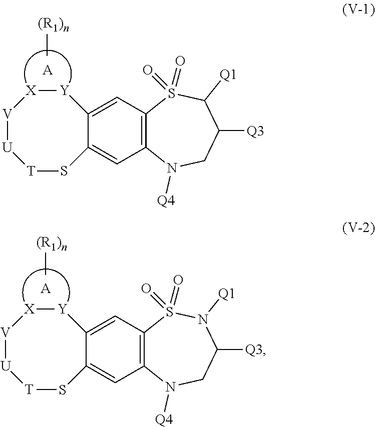
wherein Q1, Q3, Q4, S, T, U, V, X, Y, A, n and R₁ are as defined in claim 1.

3. The compound of claim 2, wherein Q1 is hydrogen or optionally substituted —C₁-C₆ alkyl; Q3 is optionally substituted —C₁-C₆ alkyl, optionally substituted —C₃-C₈ cycloalkyl, or optionally substituted 3- to 8-membered heterocycloalkyl; and Q4 is hydrogen, optionally substituted —C₁-C₆ alky, optionally substituted —C₃-C₈ cycloalkyl, optionally substituted 3- to 8-membered heterocycloalkyl, optionally substituted aryl or optionally substituted heteroaryl.

4. The compound of claim 1, represented by one of Formulas (VI-1b) to (VI-23b), or a pharmaceutically acceptable salt thereof: 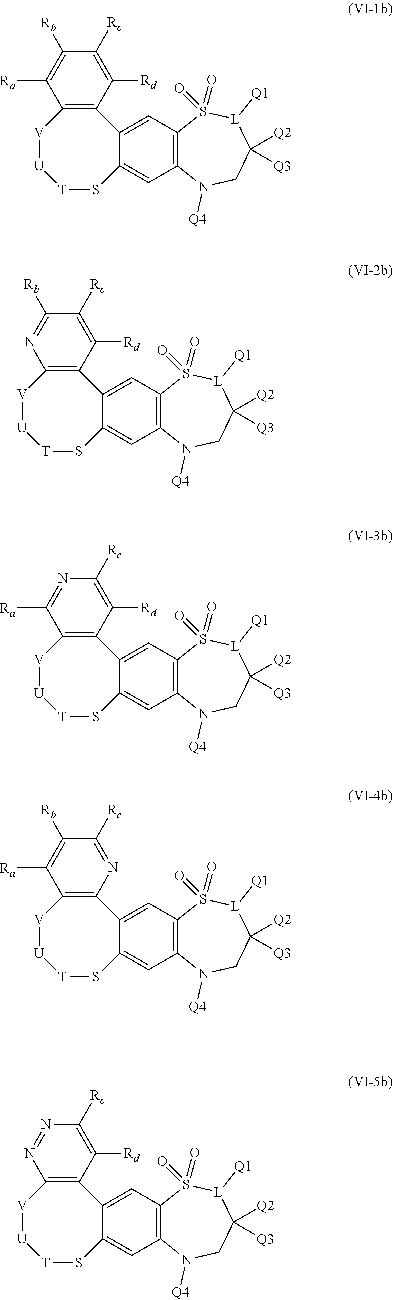 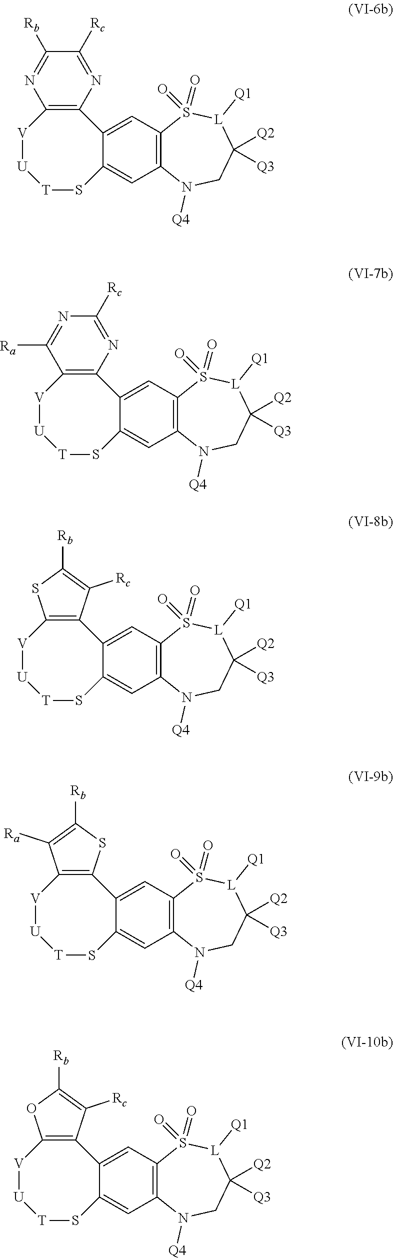 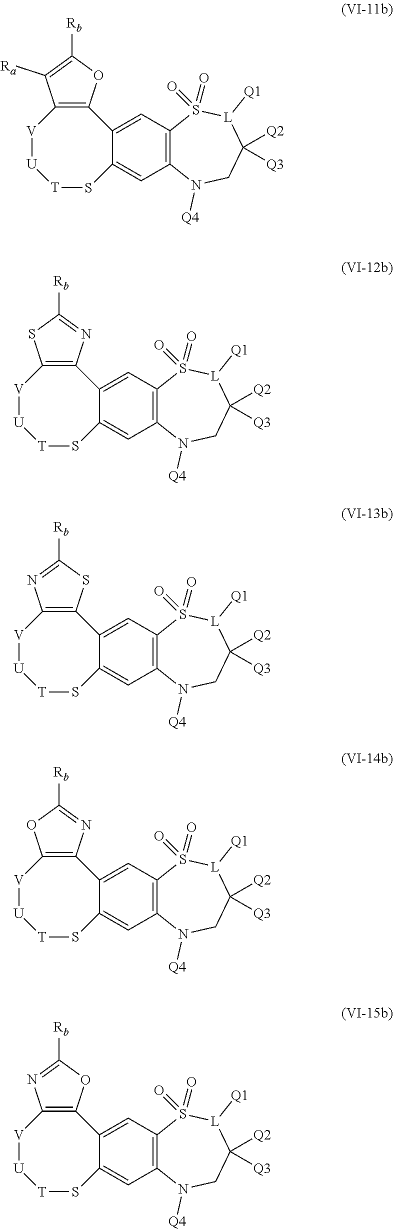 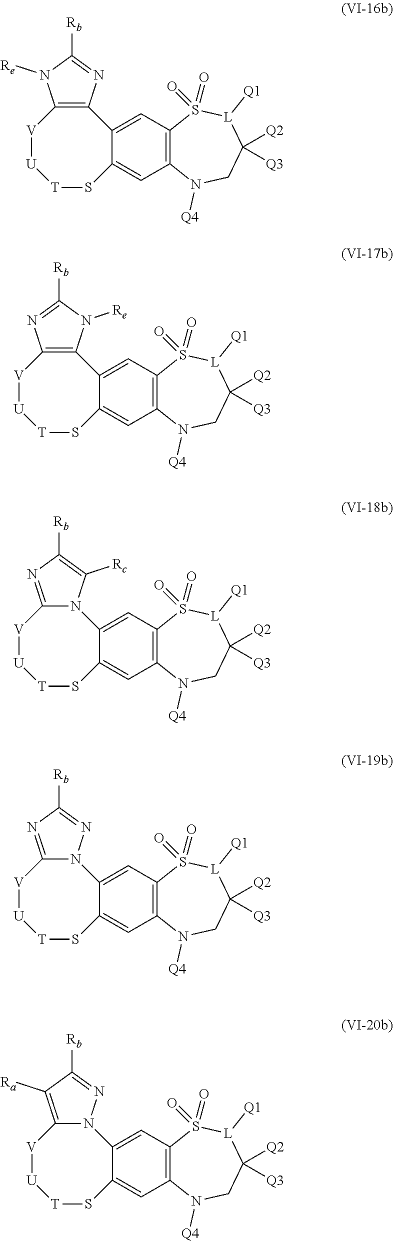 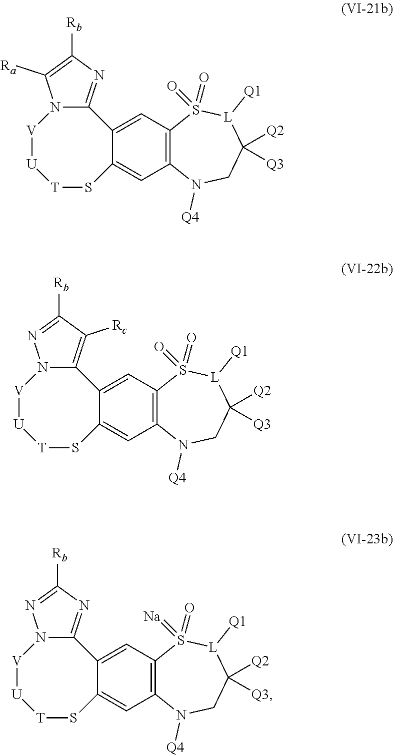
wherein Rₐ, R_{b}, R_{c}, and R_{d} are each independently selected from the group consisting of:
1) hydrogen;
2) halogen;
3) Cyano;
4) Optionally substituted —C₁-C₈ alkyl;
5) Optionally substituted —C₃-C₈ cycloalkyl;
6) —SR₂₇;
7) —SO₃H;
8) —S(O)₂R₂₇;
9) —S(O)₂N(R₂₇)(R₂₈);
10) —PO₃H₂;
11) —C(O)OR₂₇;
12) —C(O)N(R₂₇)(R₂₈);
13) —C(O)N(R₂₇)S(O)₂(R₂₈);
14) —N(R₂₇)(R₂₈);
15) —N(R₂₉)C(O)N(R₂₇)(R₂₈);
16) —N(R₂₇)C(O)(R₂₈);
17) —N(R₂₇)C(O)₂(R₂₈);
18) —N(R₂₉)S(O)₂N(R₂₇)(R₂₈);
19) —N(R₂₇)S(O)₂(R₂₈);
20) —OR₂₇; and
21) —OC(O)R₂₇;
Rₑ is selected from the group consisting of:
1) hydrogen;
2) optionally substituted —C₁-C₆ alkyl;
3) optionally substituted —C₃-C₈ cycloalkyl;
4) optionally substituted 3- to 8-membered heterocycloalkyl;
5) optionally substituted aryl; and
6) optionally substituted heteroaryl;
and L, Q1, Q2, Q3, Q4, S, T, U, V, R₂₇ and R₂₈ are as defined in claim 1.

5. The compound of claim 1, represented by one of Formulas (X-1a) to (X-4a), or a pharmaceutically acceptable salt thereof: 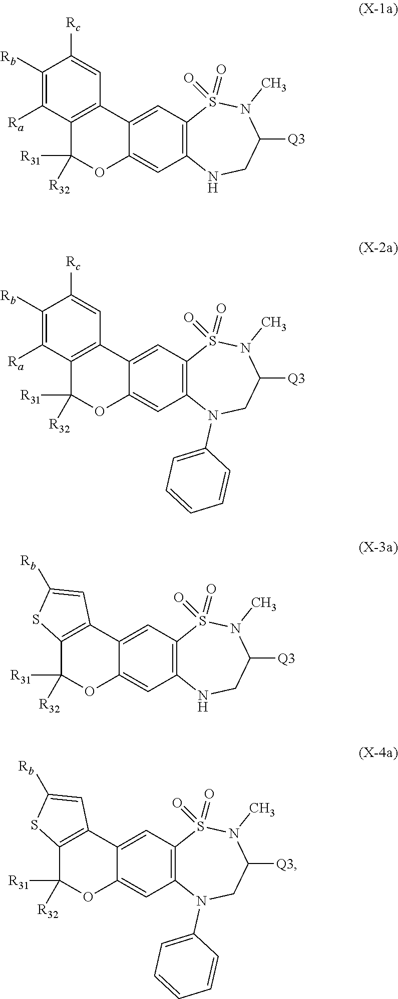
wherein Rₐ, R_{b}, and R_{c} are each independently selected from the group consisting of:
1) hydrogen;
2) halogen;
3) Cyano;
4) Optionally substituted —C₁-C₈ alkyl;
5) Optionally substituted —C₃-C₈ cycloalkyl;
6) —SR₂₇;
7) —SO₃H;
8) —S(O)₂R₂₇;
9) —S(O)₂N(R₂₇)(R₂₈);
10) —PO₃H₂;
11) —C(O)OR₂₇;
12) —C(O)N(R₂₇)(R₂₈);
13) —C(O)N(R₂₇)S(O)₂(R₂₈);
14) —N(R₂₇)(R₂₈);
15) —N(R₂₉)C(O)N(R₂₇)(R₂₈);
16) —N(R₂₇)C(O)(R₂₈);
17) —N(R₂₇)C(O)₂(R₂₈);
18) —N(R₂₉)S(O)₂N(R₂₇)(R₂₈);
19) —N(R₂₇)S(O)₂(R₂₈);
20) —OR₂₇; and
21) —OC(O)R₂₇;
R₃₁ and R₃₂ are independently selected from the group consisting of:
1) hydrogen;
2) halogen;
3) optionally substituted —C₁-C₆ alkyl;
4) optionally substituted —C₂-C₆ alkenyl
5) optionally substituted —C₃-C₈ cycloalkyl;
6) optionally substituted 3- to 8-membered heterocycloalkyl;
7) optionally substituted aryl; and
8) optionally substituted heteroaryl;
and Q3 is as defined in claim 1.

6. The compound of claim 1, represented by one of Formula Formulae (XIII-1a) (XIII-4a), or a pharmaceutically acceptable salt thereof: 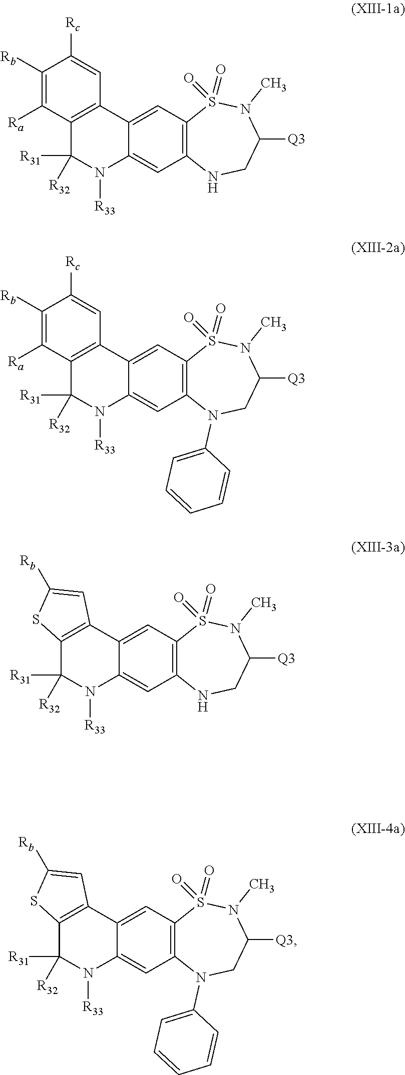
wherein Rₐ, R_{b}, and R_{c} are each independently selected from the group consisting of:
1) hydrogen;
2) halogen;
3) Cyano;
4) Optionally substituted —C₁-C₈ alkyl;
5) Optionally substituted —C₃-C₈ cycloalkyl;
6) —SR₂₇;
7) —SO₃H;
8) —S(O)₂R₂₇;
9) —S(O)₂N(R₂₇)(R₂₈);
10) —PO₃H₂;
11) —C(O)OR₂₇;
12) —C(O)N(R₂₇)(R₂₈);
13) —C(O)N(R₂₇)S(O)₂(R₂₈);
14) —N(R₂₇)(R₂₈);
15) —N(R₂₉)C(O)N(R₂₇)(R₂₈);
16) —N(R₂₇)C(O)(R₂₈);
17) —N(R₂₇)C(O)₂(R₂₈);
18) —N(R₂₉)S(O)₂N(R₂₇)(R₂₈);
19) —N(R₂₇)S(O)₂(R₂₈);
20) —OR₂₇; and
21) —OC(O)R₂₇;
R₃₁ and R₃₂ are independently selected from the group consisting of:
1) hydrogen;
2) halogen;
3) optionally substituted —C₁-C₆ alkyl;
4) optionally substituted —C₂-C₆ alkenyl
5) optionally substituted —C₃-C₈ cycloalkyl;
6) optionally substituted 3- to 8-membered heterocycloalkyl;
7) optionally substituted aryl; and
8) optionally substituted heteroaryl;
R₃₃ is independently selected from the group consisting of:
1) hydrogen;
2) optionally substituted —C₁-C₆ alkyl;
3) optionally substituted —C₂-C₆ alkenyl
4) optionally substituted —C₃-C₈ cycloalkyl;
5) optionally substituted 3- to 8-membered heterocycloalkyl;
6) optionally substituted aryl; and
7) optionally substituted heteroaryl;
and Q3 is as defined in claim 1.

7. The compound of claim 1, selected from the compounds set forth below or a pharmaceutically acceptable salt thereof:

| Com- | |
|---|---|
| pound | Structure |
| 1 | 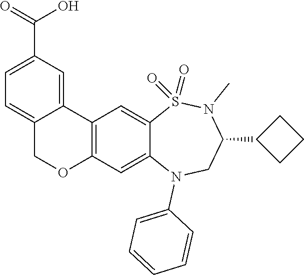 [SMILES: CN1[C@H](C2CCC2)CN(c2ccccc2)c2cc3c(cc2S1(=O)=O)-c1cc(C(=O)O)ccc1CO3] |
| 2 | 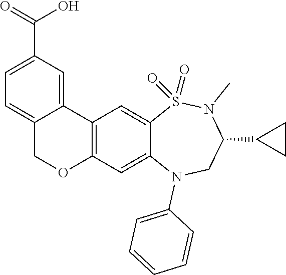 [SMILES: CN1[C@H](C2CC2)CN(c2ccccc2)c2cc3c(cc2S1(=O)=O)-c1cc(C(=O)O)ccc1CO3] |
| 3 | 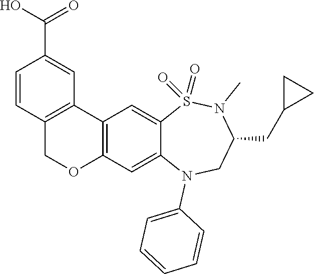 [SMILES: CN1[C@H](CC2CC2)CN(c2ccccc2)c2cc3c(cc2S1(=O)=O)-c1cc(C(=O)O)ccc1CO3] |
| 4 | 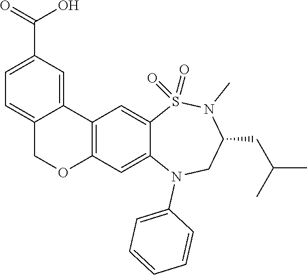 [SMILES: CC(C)C[C@@H]1CN(c2ccccc2)c2cc3c(cc2S(=O)(=O)N1C)-c1cc(C(=O)O)ccc1CO3] |
| 5 | 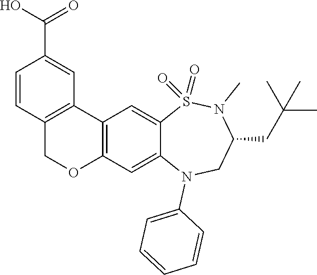 [SMILES: CN1[C@H](CC(C)(C)C)CN(c2ccccc2)c2cc3c(cc2S1(=O)=O)-c1cc(C(=O)O)ccc1CO3] |
| 6 | 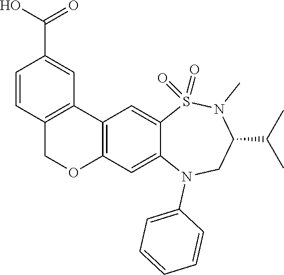 [SMILES: CC(C)[C@@H]1CN(c2ccccc2)c2cc3c(cc2S(=O)(=O)N1C)-c1cc(C(=O)O)ccc1CO3] |
| 7 | 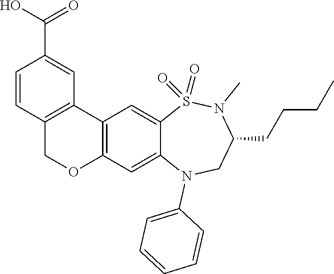 [SMILES: CCCC[C@@H]1CN(c2ccccc2)c2cc3c(cc2S(=O)(=O)N1C)-c1cc(C(=O)O)ccc1CO3] |
| 8 | 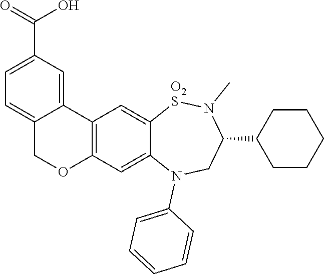 [SMILES: CN1[C@H](C2CCCCC2)CN(c2ccccc2)c2cc3c(cc2S1(=O)=O)-c1cc(C(=O)O)ccc1CO3] |
| 9 | 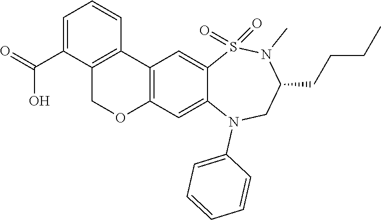 [SMILES: CCCC[C@@H]1CN(c2ccccc2)c2cc3c(cc2S(=O)(=O)N1C)-c1cccc(C(=O)O)c1CO3] |
| 10 | 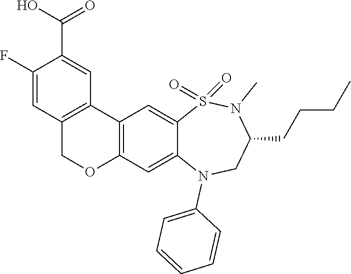 [SMILES: CCCC[C@@H]1CN(c2ccccc2)c2cc3c(cc2S(=O)(=O)N1C)-c1cc(C(=O)O)c(F)cc1CO3] |
| 11 | 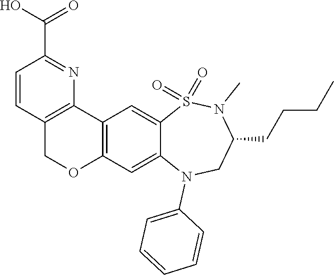 [SMILES: CCCC[C@@H]1CN(c2ccccc2)c2cc3c(cc2S(=O)(=O)N1C)-c1nc(C(=O)O)ccc1CO3] |
| 12 | 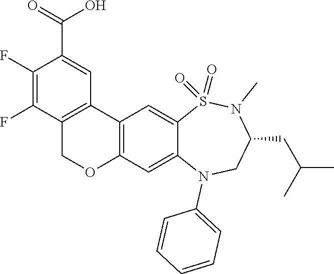 [SMILES: CC(C)C[C@@H]1CN(c2ccccc2)c2cc3c(cc2S(=O)(=O)N1C)-c1cc(C(=O)O)c(F)c(F)c1CO3] |
| 13 | 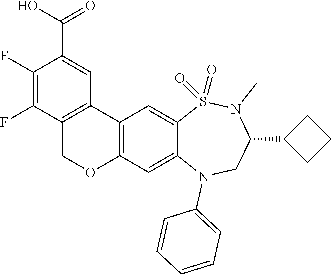 [SMILES: CN1[C@H](C2CCC2)CN(c2ccccc2)c2cc3c(cc2S1(=O)=O)-c1cc(C(=O)O)c(F)c(F)c1CO3] |
| 14 | 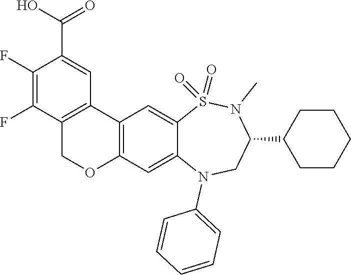 [SMILES: CN1[C@H](C2CCCCC2)CN(c2ccccc2)c2cc3c(cc2S1(=O)=O)-c1cc(C(=O)O)c(F)c(F)c1CO3] |
| 15 | 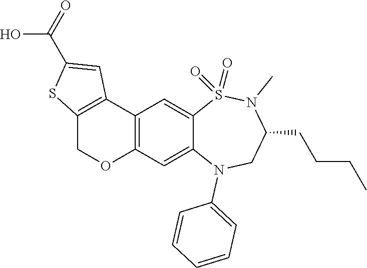 [SMILES: CCCC[C@@H]1CN(c2ccccc2)c2cc3c(cc2S(=O)(=O)N1C)-c1cc(C(=O)O)sc1CO3] |
| 16 | 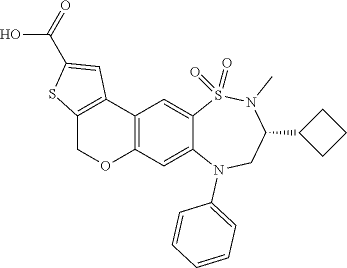 [SMILES: CN1[C@H](C2CCC2)CN(c2ccccc2)c2cc3c(cc2S1(=O)=O)-c1cc(C(=O)O)sc1CO3] |
| 17 | 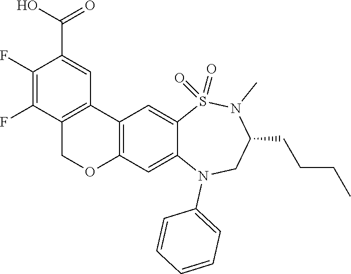 [SMILES: CCCC[C@@H]1CN(c2ccccc2)c2cc3c(cc2S(=O)(=O)N1C)-c1cc(C(=O)O)c(F)c(F)c1CO3] |
| 18 | 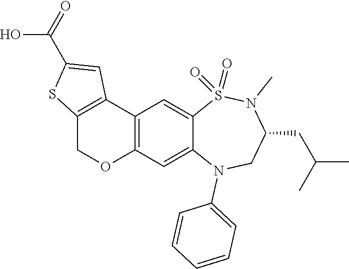 [SMILES: CC(C)C[C@@H]1CN(c2ccccc2)c2cc3c(cc2S(=O)(=O)N1C)-c1cc(C(=O)O)sc1CO3] |
| 19 | 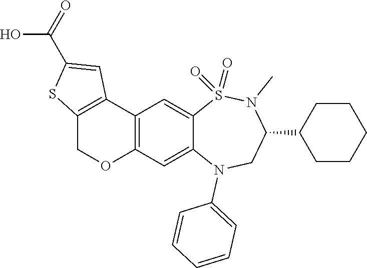 [SMILES: CN1[C@H](C2CCCCC2)CN(c2ccccc2)c2cc3c(cc2S1(=O)=O)-c1cc(C(=O)O)sc1CO3] |
| 20 | 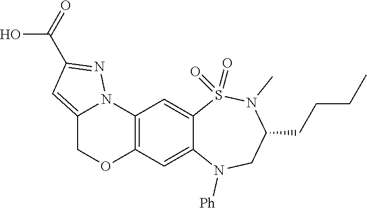 [SMILES: CCCC[C@@H]1CN(c2ccccc2)c2cc3c(cc2S(=O)(=O)N1C)-n1nc(C(=O)O)cc1CO3] |
| 21a | 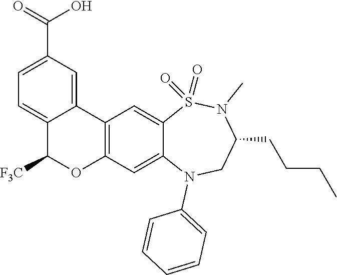 [SMILES: CCCC[C@@H]1CN(c2ccccc2)c2cc3c(cc2S(=O)(=O)N1C)-c1cc(C(=O)O)ccc1[C@H](C(F)(F)F)O3] |
| 21b | 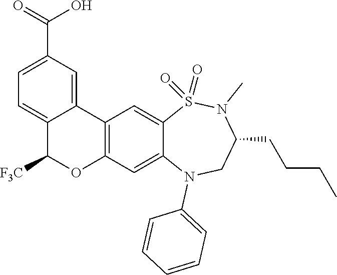 [SMILES: CCCC[C@@H]1CN(c2ccccc2)c2cc3c(cc2S(=O)(=O)N1C)-c1cc(C(=O)O)ccc1[C@H](C(F)(F)F)O3] |
| 22a | 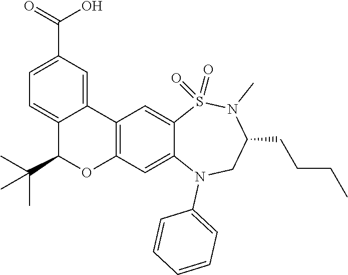 [SMILES: CCCC[C@@H]1CN(c2ccccc2)c2cc3c(cc2S(=O)(=O)N1C)-c1cc(C(=O)O)ccc1[C@H](C(C)(C)C)O3] |
| 22b | 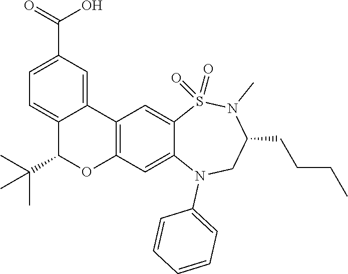 [SMILES: CCCC[C@@H]1CN(c2ccccc2)c2cc3c(cc2S(=O)(=O)N1C)-c1cc(C(=O)O)ccc1[C@@H](C(C)(C)C)O3] |
| 23 | 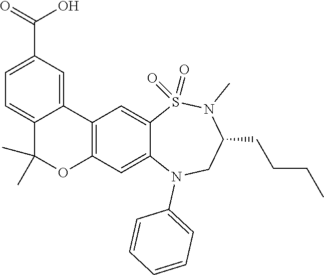 [SMILES: CCCC[C@@H]1CN(c2ccccc2)c2cc3c(cc2S(=O)(=O)N1C)-c1cc(C(=O)O)ccc1C(C)(C)O3] |
| 24 | 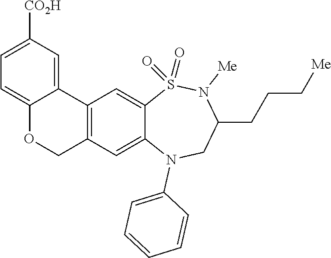 [SMILES: CCCCC1CN(c2ccccc2)c2cc3c(cc2S(=O)(=O)N1C)-c1cc(C(=O)O)ccc1OC3] |
| 25 | 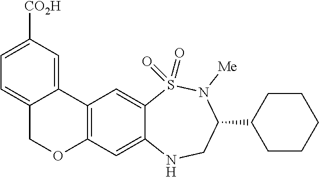 [SMILES: CN1[C@H](C2CCCCC2)CNc2cc3c(cc2S1(=O)=O)-c1cc(C(=O)O)ccc1CO3] |
| 26 | 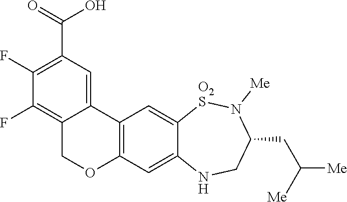 [SMILES: CC(C)C[C@@H]1CNc2cc3c(cc2S(=O)(=O)N1C)-c1cc(C(=O)O)c(F)c(F)c1CO3] |
| 27 | 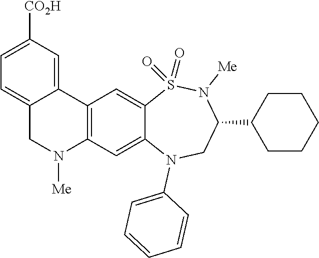 [SMILES: CN1Cc2ccc(C(=O)O)cc2-c2cc3c(cc21)N(c1ccccc1)C[C@@H](C1CCCCC1)N(C)S3(=O)=O] |
| 28 | 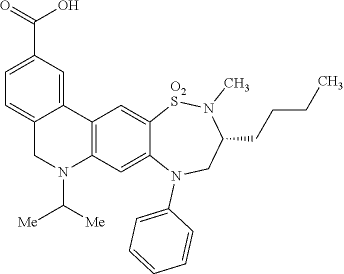 [SMILES: CCCC[C@@H]1CN(c2ccccc2)c2cc3c(cc2S(=O)(=O)N1C)-c1cc(C(=O)O)ccc1CN3C(C)C] |
| 29 | 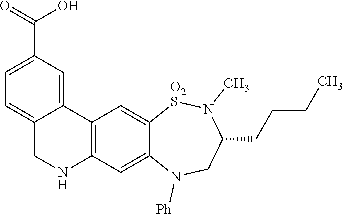 [SMILES: CCCC[C@@H]1CN(c2ccccc2)c2cc3c(cc2S(=O)(=O)N1C)-c1cc(C(=O)O)ccc1CN3] |
| 30 | 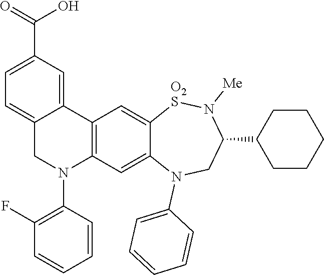 [SMILES: CN1[C@H](C2CCCCC2)CN(c2ccccc2)c2cc3c(cc2S1(=O)=O)-c1cc(C(=O)O)ccc1CN3c1ccccc1F] |
| 31 | 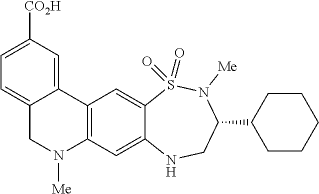 [SMILES: CN1Cc2ccc(C(=O)O)cc2-c2cc3c(cc21)NC[C@@H](C1CCCCC1)N(C)S3(=O)=O] |
| 32 | 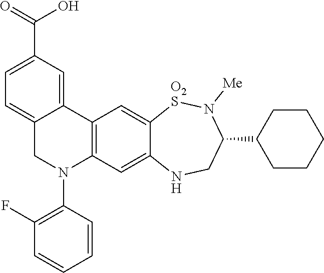 [SMILES: CN1[C@H](C2CCCCC2)CNc2cc3c(cc2S1(=O)=O)-c1cc(C(=O)O)ccc1CN3c1ccccc1F] |
| 33 | 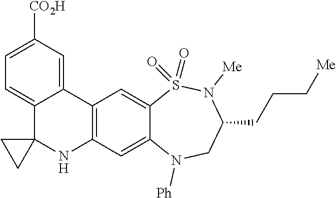 [SMILES: CCCC[C@@H]1CN(c2ccccc2)c2cc3c(cc2S(=O)(=O)N1C)-c1cc(C(=O)O)ccc1C1(CC1)N3] |
| 34 | 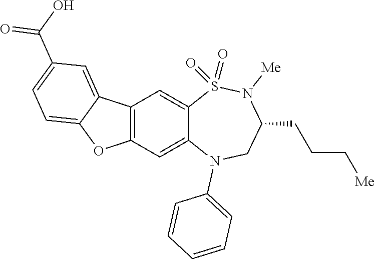 [SMILES: CCCC[C@@H]1CN(c2ccccc2)c2cc3oc4ccc(C(=O)O)cc4c3cc2S(=O)(=O)N1C] |
| 35 | 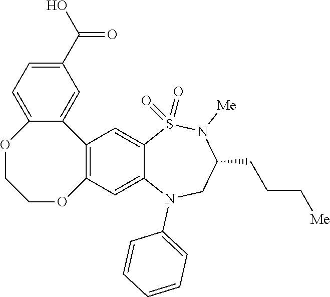 [SMILES: CCCC[C@@H]1CN(c2ccccc2)c2cc3c(cc2S(=O)(=O)N1C)-c1cc(C(=O)O)ccc1OCCO3] |
| 36 | 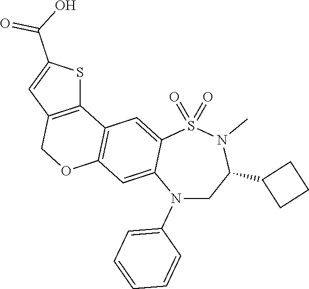 [SMILES: CN1[C@H](C2CCC2)CN(c2ccccc2)c2cc3c(cc2S1(=O)=O)-c1sc(C(=O)O)cc1CO3] |
| 37 | 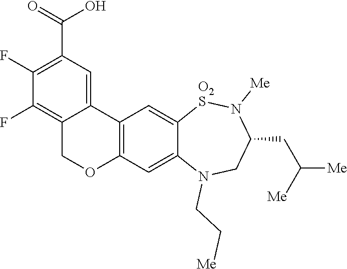 [SMILES: CCCN1C[C@@H](CC(C)C)N(C)S(=O)(=O)c2cc3c(cc21)OCc1c-3cc(C(=O)O)c(F)c1F] |
| 38 | 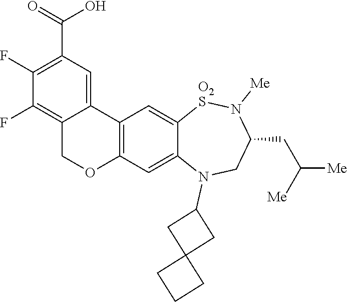 [SMILES: CC(C)C[C@@H]1CN(C2CC3(CCC3)C2)c2cc3c(cc2S(=O)(=O)N1C)-c1cc(C(=O)O)c(F)c(F)c1CO3] |
| 39 | 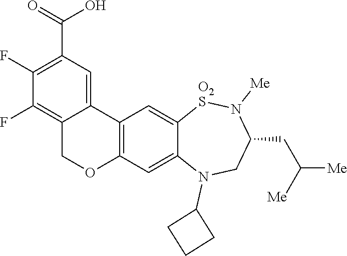 [SMILES: CC(C)C[C@@H]1CN(C2CCC2)c2cc3c(cc2S(=O)(=O)N1C)-c1cc(C(=O)O)c(F)c(F)c1CO3] |
| 40 | 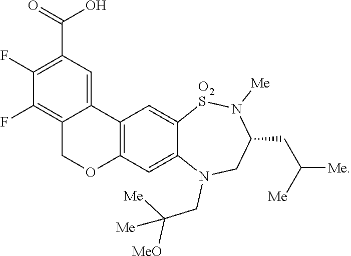 [SMILES: COC(C)(C)CN1C[C@@H](CC(C)C)N(C)S(=O)(=O)c2cc3c(cc21)OCc1c-3cc(C(=O)O)c(F)c1F] |

8. A pharmaceutical composition, comprising a compound according to claim 1, in combination with a pharmaceutically acceptable carrier or excipient.

9. A method of treating or preventing an HBV and/or HDV infection in a subject in need thereof, comprising administering to the subject a therapeutically effective amount of a compound or a combination of compounds according to claim 1.

10. The method of claim 9, further comprising administering to the subject an additional therapeutic agent selected from the group consisting of a HBV polymerase inhibitor, interferon, viral entry inhibitor, viral maturation inhibitor, literature-described capsid assembly modulator, reverse transcriptase inhibitor, TLR-agonist, inducer of cellular viral RNA sensor, therapeutic vaccine, and agents of distinct or unknown mechanism, and a combination thereof.

11. The method of claim 10, wherein the compound and the additional therapeutic agent are co-formulated.

12. The method of claim 10, wherein the compound and the additional therapeutic agent are co-administered.

13. The method of claim 10, wherein the additional therapeutic agent is administered at a lower dose or frequency compared to the dose or frequency of the additional therapeutic agent that is required to treat an HBV and/or HDV infection when administered alone.

14. The method of claim 10, wherein the subject is refractory to at least one compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

15. The method of claim 10, wherein the method reduces viral load in the subject to a greater extent compared to the administering of a compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

16. The method of claim 10, wherein the method results in a lower incidence of viral mutation and/or viral resistance than the treatment with a compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.
